(19) United States
(12) Patent Application Publication
(10) Pub. No.: US 2003/0224991 A1
(43) Pub. Date: Dec. 4, 2003

(54) Nucleotide sequences and amino acid sequences of secreted proteins involved in angiogenesis

(76) Inventors: Mohanraj Dhanabal, Branford, CT (US); Frank Wu, Woodbridge, CT (US); William J. LaRochelle, Madison, CT (US); Henri S. Lichenstein, Guilford, CT (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/385,802

(22) Filed: Mar. 11, 2003

Related U.S. Application Data
(60) Provisional application No. 60/363,266, filed on Mar. 11, 2002.

(51) Int. Cl. A61K 38/36; A61K 48/00; C12Q 1/68; C07H 21/04; C12P 21/02; C12N 5/06

(52) U.S. Cl. 514/12; 514/44; 435/6; 435/69.1; 435/320.1; 435/325; 530/350; 536/23.2

(57) ABSTRACT

Novel anti-angiogenesis proteins and the nucleic acid sequences which encode them are disclosed by the present invention.

### RELATED APPLICATIONS

This application claims priority to U.S. Ser. No. 60/363,266, filed Mar. 11, 2002. The contents of this application are incorporated by reference in their entirety.

### FIELD OF THE INVENTION

The invention relates generally to nucleic acids and the polypeptides encoded by these nucleic acids that are angiogenesis-modulating polypeptides, fragments, fusion proteins and and methods of use thereof.

### BACKGROUND OF THE INVENTION

Under normal physiological conditions, humans or animals undergo angiogenesis, i.e., generation of new blood vessels into a tissue or organ, only in restricted situations. During angiogenesis, endothelial cells react to stimulation with finely tuned signaling responses. The “endothelium” is a thin layer of flat epithelial cells that lines serous cavities, lymph vessels, and blood vessels. In normal physiological states such as embryonic growth and wound healing, neovascularization is controlled by a balance of stimulatory and inhibitory angiogenic factors. These controls may fail and result in formation of an extensive capillary network during the development of many diseases including ischemic heart disease, ischemic peripheral vascular disease, tumor growth and metastasis, reproduction, embryogenesis, wound healing, bone repair, rheumatoid arthritis, diabetic retinopathy and other diseases.

Both controlled and uncontrolled angiogenesis are thought to proceed in a similar manner. Endothelial cells and pericytes, surrounded by a basement membrane, form capillary blood vessels. Angiogenesis begins with the erosion of the basement membrane by enzymes released by endothelial cells and leukocytes. The endothelial cells, which line the lumen of blood vessels, then protrude through the basement membrane. Angiogenic stimulants induce the endothelial cells to migrate through the eroded basement membrane. The migrating cells form a “sprout” off the parent blood vessel, where the endothelial cells undergo mitosis and proliferate. The endothelial sprouts merge with each other to form capillary loops, creating the new blood vessel.

Persistent, unregulated angiogenesis occurs in a multiplicity of disease states, tumor metastasis and abnormal growth by endothelial cells and supports the pathological damage seen in these conditions. The diverse pathological disease states in which unregulated angiogenesis is present have been grouped together as angiogenic dependent or angiogenic associated diseases.

The balance of positive or negative angiogeflesis regulators control the fate of vascular wall cells. They remain either in a state of vascular homeostasis, or they proceed to neovascularization, e.g., tumor growth and the switch to an angiogenic tumor phenotype correlates with increased secretion of angiogenic molecules such as fibroblast growth factor (FGF), vascular endothelial growth factor (VEGF), and others. On the other hand, tumors also acquire a more angiogenic phenotype because inhibitors of angiogenesis are down-regulated during tumorigenesis (e.g. thrombospondin) (Dameron et al., 1994, Science 265:1582-1584).

Angiogenesis has been implicated in various cancers. Angiogenesis is an essential component of the metastatic pathway (see, e.g. Zetter, 1998, Ann. Rev. Med. 49:407-427). These blood vessels provide the principal pathway by which tumor cells exit the primary tumor site and enter the circulation. Tumor angiogenesis is regulated by the production of angiogenic stimulators including members of the FGF and VEGF families (see, e.g. Femig & Gallaher, 1994, Prog. Growth Factor Res. 5:353-377). Tumors may also activate angiogenic inhibitors such as angiostatin (U.S. Pat. No. 5,639,725, herein incorporated by reference) and endostatin that can modulate angiogenesis both at the primary site and at downstream sites of metastasis. The potential use of these and other natural and synthetic angiogenic inhibitors as anticancer drugs is currently under intense investigation (see, e.g. Zetter, 1998, Ann. Rev. Med. 49:407-427). Such agents may have reduced toxicity and be less likely to generate drug resistance than conventional cytotoxic drugs. Clinical trials are now underway to develop optimum treatment strategies for antiangiogenic agents.

Angiopoietin-1 (Ang-1) is an angiogenic factor that signals through the endothelial cell-specific Tie2 receptor tyrosine kinase. Like VEGF, Ang-1 is essential for normal endothelial developmental processes in the mouse (Davis et al., 1996, Cell 87:). Furthermore, Ang-1 induces the formation of capillary sprouts (Koblizek et al., 1998, Curr. Biol. 8:529-532). The protein is expressed only on endothelial cells and early hemopoietic cells (e.g., see Sure et al., 1996, Cell 87:1171-1180).

Angiopoietin-2 (Ang-2) is a naturally occurring antagonist for Ang1 and Tie2 and can disrupt blood vessel formation in the mouse embryos (see, eg. Maisonpierre et al., 1997, Science 277:55-). Ang-2 is expressed only at sites of vascular remodeling.

Angiogenic and antiangiogenic (or angiostatic) molecules control the formation of new vessels via different mechanisms. Antiangiogenic molecules, or angiogenesis inhibitors (e.g. angiostatin, endostatin, angiopoeitin-1 (Ang11), rat microvascular endothelial differentiation gene (MEDG), somatostatin, thrombospondin-2 (TSP-2), platelet factor-4(PF-4), 16-kDa N terminal fragment of prolactin and maspin can repress angiogenesis, and therefore, maintain vascular homeostasis (see, e.g. for review Bicknell, 1994, Ann. Oncol. 5 (suppl) 4:45-50; Jouan, V., et al. (1999). Blood 94, 984-993; O'Reilly, M. S., et al. (1997). Cell 88, 277-285; Ferrara, N., et al.. (1991). Endocrinology 129, 896-900; Zhang, M., et al. (2000). Nat. Med. 6, 196-199).

Angiopoietins (Ang 1, Ang 2) are ˜70-kDa proteins that share considerable sequence homology. Each protein consists of a signal peptide, an NH₂-terminal coiled-coil domain, a short linker peptide region and a COOH-terminal fibrinogen homology domain (FD). The coiled-coil region is responsible for dimerization of angiopoietin and the FD binds to Tie2 receptors. Both Ang 1 and Ang 2 form dimers and oligomers (Procopio, W. N., et al. (1999). J. Biol. Chem. 274, 30196-30201.; Suri, C.,et al. (1996). Cell 87, 1171-1180). In vivo analysis by targeted gene disruption reveals that Ang 1 recruits and sustains peri-endothelial support cells (Davis, S., et al. (1996). Cell 87, 1161-1169; Sato, A., et al.. (1998). Int. Immunol. 10, 1217-1227; Suri et al., 1996), whereas, Ang 2 disrupts blood vessel formation in the developing embryo by antagonizing the effect of Ang 1 on the Tie2 receptor (Maisonpierre, P. C., et al. (1997). Science 277, 55-60). Later, Ang 4 was shown to be a third protein capable of binding to the Tie2 receptor (Valenzuela, D. M., et al. (1999). Proc. Natl. Acad. Sci. U.S.A. 96, 1904-1909). Three additional proteins (ARP1, ARP2 and CDT6) with similarity to angiopoietins have also been discovered (Kim, I., et al. (1999a). FEBS Lett. 443, 353-356; Kim, I., et al. (1999b). J. Biol. Chem. 274, 26523-26528; Peek, R., et al. (2001). J. Biol. Chem. 26, 26; Peek, R., et al. (1998). Invest. Ophthalmol. Vis. Sci. 39, 1782-1788). However, these proteins do not bind to Tie2 or related Tie1 receptor and do not possess a specific cysteine motif that is characteristic of angiopoietins (Valenzuela et al., 1999).

In animal models some angiogenesis-dependent diseases can be controlled via inhibition of new vessel formation. Treatment of diseases by modulation of angiogenesis are currently tested in clinical trials. Thus the manipulation of new vessel formation in angiogenesis-dependent conditions such as wound healing, inflammatory diseases, ischemic heart and peripheral vascular disease, myocardial infarction, diabetic retinopathy, and cancer is likely to create new therapeutic options.

Angiogenesis is believed to play a significant role in the metastasis of a cancer. If this angiogenic activity could be repressed or eliminated, then the tumor, although present, would not grow. In the disease state, prevention of angiogenesis could avert the damage caused by the invasion of the new microvascular system. Therapies directed at control of the angiogenic processes could lead to the abrogation or mitigation of these diseases. Novel antiangiogenic molecules are needed, both to model unwanted growth of blood vessels, especially into tumors, and for therapies directed to preventing such unwanted growth.

### SUMMARY OF THE INVENTION

The present invention is directed to novel molecules, referred to herein as Angioarrestin polypeptides, as well as nucleic acid sequences encoding those molecules. Processes are also provided for producing a protein, which comprise growing a culture of host cells producing such proteins (as described above) in a suitable culture medium, and purifying the protein from the culture. The protein produced according to such methods is also provided by the present invention. The present invention is also directed to Angioarrestin protein fragments, fusion proteins, and methods of use thereof

In one embodiment, the invention involves an isolated nucleic acid molecule comprising a nucleic acid sequence encoding a polypeptide having an amino acid sequence selected from the group consisting of SEQ ID NOs: 2, 8, 10, 12, 18, 24, 26, 28, 30, 32, 34, 36, 38, 40, 42, 44, 46, 48, 50, 52, 54, 56, 58, 60, 62, 64, 70, 72, a nucleic acid fragment encoding at least a portion of a polypeptide comprising the amino acid sequence selected from the group consisting of SEQ ID NOs: 2, 8, 10, 12, 18, 24, 26, 28, 30, 32, 34, 36, 38, 40, 42, 44, 46, 48, 50, 52, 54, 56, 58, 60, 62, 64, 70, 72, and the complement of any of the nucleic acid molecules.

In another embodiment, the invention includes an isolated nucleic acid molecule having a nucleic acid sequence encoding a polypeptide including an amino acid sequence selected from the group consisting of SEQ ID NOs: 2, 8, 10, 12, 18, 24, 26, 28, 30, 32, 34, 36, 38, 40, 42, 44, 46, 48, 50, 52, 54, 56, 58, 60, 62, 64, 70, 72, wherein the nucleic acid molecule comprises a nucleotide sequence selected from the group consisting of SEQ ID NOs: 1, 7, 9, 11, 17, 23, 25, 27, 29, 31, 33, 35, 37, 39, 41, 43, 45, 47, 49, 51, 53, 55, 57, 59, 61, 69, 71.

In another embodiment, the invention includes an isolated nucleic acid molecule having a nucleic acid sequence encoding a polypeptide including an amino acid sequence selected from the group consisting SEQ ID NOs: 2, 8, 10, 12, 18, 24, 26, 28, 30, 32, 34, 36, 38, 40, 42, 44, 46, 48, 50, 52, 54, 56, 58, 60, 62, 64, 70, 72, wherein the nucleic acid molecule hybridizes under stringent conditions to the nucleotide sequence selected from the group consisting of of SEQ ID NOs: 1, 7, 9, 11, 17, 23, 25, 27, 29, 31, 33, 35, 37, 39, 41, 43, 45, 47, 49, 51, 53, 55, 57, 59, 61, 69, 71, or a complement of the nucleotide sequence.

In another embodiment, the invention includes an isolated nucleic acid molecule having a nucleic acid sequence encoding a polypeptide including an amino acid sequence selected from the group consisting of SEQ ID NOs: 2, 8, 10, 12, 18, 24, 26, 28, 30, 32, 34, 36, 38, 40, 42, 44, 46, 48, 50, 52, 54, 56, 58, 60, 62, 64, 70, 72, an isolated second polynucleotide that is a complement of the first polynucleotide, or a fragment of any of them.

In another embodiment the invention includes an isolated nucleic acid molecule capable of hybridizing under stringent conditions to the nucleiotide sequence selected form the group consisting of SEQ ID NOs: 1, 7, 9, 11, 17, 23, 25, 27, 29, 31, 33, 35, 37, 39, 41, 43, 45, 47, 49, 51, 53, 55, 57, 59, 61, 69, 71. In another embodiment the invention includes an isolated nucleic acid molecule capable of hybridizing under stringent conditions to the nucleiotide sequence encoding a polypeptide with an amino acid sequence selected from the group consisting of SEQ ID NOs: 2, 8, 10, 12, 18, 24, 26, 28, 30, 32, 34, 36, 38, 40, 42, 44, 46, 48, 50, 52, 54, 56, 58, 60, 62, 64, 70, 72.

In certain preferred embodiments, the novel nucleic acid sequences of this invention are operatively linked to one or more expression control sequences. The invention also provides a host cell, including bacterial, plant, yeast, insect and mammalian cells, that produce the novel polypeptides, whether the cell is transformed with the nucleic acid sequences encoding those proteins, or whether the cell is transformed with regulatory sequences to activate or enhance production of these proteins from an endogenous nucleic acid sequence encoding same.

In another embodiment, the invention includes a vector including the nucleic acid molecule having a nucleic acid sequence encoding a polypeptide including an amino acid sequence selected from the group consisting of SEQ ID NOs: 2, 8, 10, 12, 18, 24, 26, 28, 30, 32, 34, 36, 38, 40, 42, 44, 46, 48, 50, 52, 54, 56, 58, 60, 62, 64, 70, 72. The invention also includes a vector including the nucleic acid molecule having a nucleic acid sequence encoding a polypeptide, the nucleic acid sequence selected from the group consisting of of SEQ ID NOs: 1, 7, 9, 11, 17, 23, 25, 27, 29, 31, 33, 35, 37, 39, 41, 43, 45, 47, 49, 51, 53, 55, 57, 59, 61, 69, 71. This vector can have a promoter operably linked to the nucleic acid molecule. This vector can be located within a cell.

Another embodiment of the invention includes the polypeptides comprising amino acid sequences SEQ ID NOs: 2, 8, 10, 12, 18, 24, 26, 28, 30, 32, 34, 36, 38, 40, 42, 44, 46, 48, 50, 52, 54, 56, 58, 60, 62, 64, 70, 72, or fragments thereof, the protein being substantially free from other mammalian proteins. Compositions comprising an antibody which specifically reacts with such protein are also contemplated by the present invention. Methods are also contemplated for preventing, treating or ameliorating a medical condition which comprises administering to a mammalian subject a therapeutically effective amount of a composition comprising a protein of the present invention and a pharmaceutically acceptable carrier.

Compositions of the invention including polypeptides may further comprise a pharmaceutically acceptable carrier. In another embodiment, the invention comprises a pharmaceutical composition involving a polypeptide with an amino acid sequence selected from the group consisting of SEQ ID NOs: 2, 8, 10, 12, 18, 24, 26, 28, 30, 32, 34, 36, 38, 40, 42, 44, 46, 48, 50, 52, 54, 56, 58, 60, 62, 64, 70, 72, and a pharmaceutically acceptable carrier. In another embodiment, the invention provides a kit, including, in one or more containers, the pharmaceutical composition.

In another embodiment, the invention includes the use of a therapeutic in the manufacture of a medicament for treating a angiogenic related syndromes associated with a human disease.

In another embodiment: the invention involves a method of treating a tumor or preventing its growth by inhibiting angiogenesis using a polypeptide with an amino acid sequence selected from the group consisting of SEQ ID NOs: 2, 8, 10, 12, 18, 24, 26, 28, 30, 32, 34, 36, 38, 40, 42, 44, 46, 48, 50, 52, 54, 56, 58, 60, 62, 64, 70, 72, the method including administering the polypeptide to a subject in an amount sufficient to treat or prevent tumor growth.

The proteins disclosed in this invention play a role in angiogenesis. Accordingly, the compositions and methods of this invention are useful in anti-tumor or anti-cancer therapies. Diagnostic, prognostic and screening kits are also contemplated. In certain antiangiogenic embodiments, the compositions and methods of this invention are useful in inhibiting the activity of endogenous growth factors in premetastatic and metastatic tumors and preventing the formation of the capillaries in the tumors thereby inhibiting the growth of the tumors. The composition, and antibodies specific to the composition, should also be able to modulate the formation of capillaries in other angiogenic processes, such as wound healing and reproduction. Finally, the composition and method for inhibiting angiogenesis should preferably be non-toxic and produce few side effects.

Certain Angiopoietin proteins require a coiled coil domain in order to form dimmer, trimer or oligomers and a fibrinogen binding domain (FBD) to activate receptor binding signal transduction. The current invention preferably utilizes uniquely identified sub-domains of a coiled coil domain, referred to herein as coiled coil 1 (CC1) polynucleotide SEQ ID NO:69 encoding polypeptide SEQ ID NO:70, and coiled coil 2 (CC2) polynucleotide SEQ ID NO:71 encoding polypeptide SEQ ID NO:72. One coiled coil sub domain, either CC1 or CC2 is shown herein to be sufficient for activity of the Angioarrestin proteins of the invention.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In the case of conflict, the present Specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

FIG. 1 is a histogram showing the effects of CG57067-08 on the proliferation of human vascular endothelial cells (HUVEC).

FIG. 2 is a histogram showing the effects of CG57067-08 on the migration of human vascular endothelial cells (HUVEC).

FIG. 3 is a histogram showing the effects of CG57067-08 on the adhesion of human vascular endothelial cells (HUVEC).

FIG. 4 is a histogram showing the effects of CG57067-08 on human RCC cells (786-0) induced angiogenesis (nodes) in matrigel in vivo assay.

FIG. 5 is a histogram showing the effects of CG57067-08 on human RCC cells (780) induced angiogenesis (vessels) in matrigel in vivo assay.

FIG. 6 is a histogram showing the effects of CG57067-08 on human RCC cells (780) induced angiogenesis (length) in matrigel in vivo assay.

FIG. 7 is a histogram showing the effects of CG57067-19 on migration of human vascular endothelial cells (HUVEC).

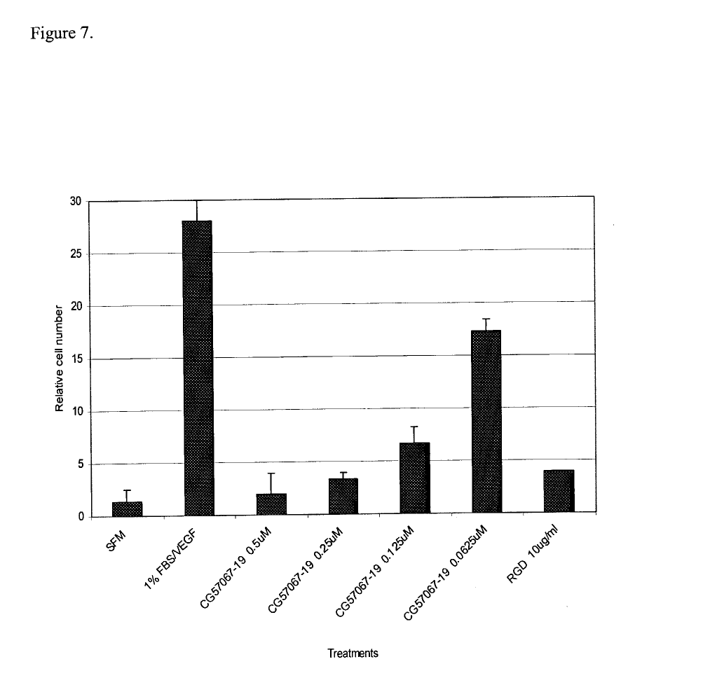

FIG. 8 is a histogram showing the effects of CG57067-19 on migration of human pancreatic carcinoma cells (Panc-1).

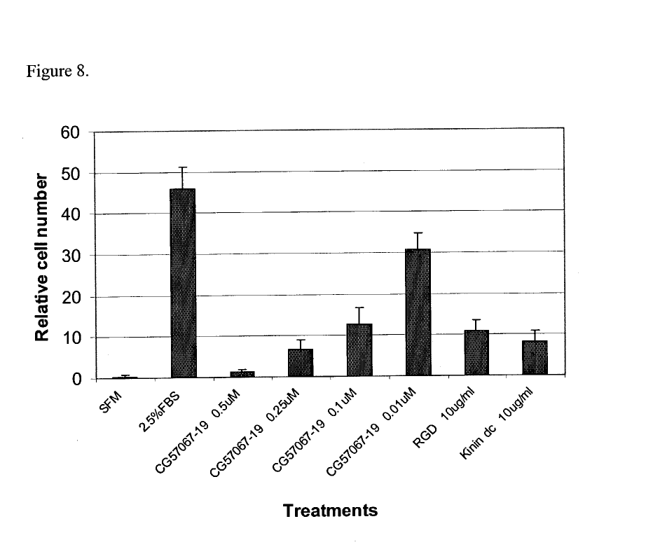

FIG. 9 is a histogram showing the effects of CG57067-19 on human RCC cells (786-0) induced angiogenesis (nodes) in matrigel in vivo assay.

FIG. 10 is a histogram showing the effects of CG57067-19 on human RCC cells (780) induced angiogenesis (vessels) in matrigel in vivo assay.

FIG. 11 is a histogram showing the effects of CG57067-19 on human RCC cells (780) induced angiogenesis (length) in matrigel in vivo assay.

### DETAILED DESCRIPTION OF THE INVENTION

The invention provides for novel Angioarrestin proteins and genes encoding those proteins, as well as derivatives, homologs, active fragments and analogs, from various species, particularly vertebrates, and more particularly mammals. In some embodiments, the polypeptides modulate antiangiogenic activites. Antiangiogenic molecules of the invention are molecules that elicits an effect on angiogenesis in vivo upon exogenous administration or overexpression, that has an effect on relevant endothelial cells in vitro that is compatible with angiogenesis/antiangiogenesis, and the role of the molecule has been established in a process or disease.

In a preferred embodiment, the foregoing proteins and genes are of human origin. Production of the foregoing proteins and derivatives, e.g., by recombinant methods, is also contemplated in the present invention. In other specific embodiments, the fragment, derivative or analog is functionally active, i.e., capable of exhibiting one or more functional activity associated with Angioarrestin. Such functional activities include, but are not limited to, the stimulation or inhibition of angiogenesis and related disorders. Such functional activities include further, but are not limited to, antigenicity or ability to bind (or compete with Angioarrestin) to an anti-Angioarrestin, antibody immunogenicity (ability to generate an antibody that binds to anti-Angioarrestin), etc.

The invention provides novel Angioarrestin polypeptides and the nucleic acids encoding them, as outlined in Table 1. Novel Angioarrestin encoding nucleic acid molecules include the polynucleotide sequences set forth in Table 2. The amino acid sequences of the proteins encoded by Angioarrestin polynucleotides are also shown in Table 2.

TABLE 1
Sequences and Corresponding SEQ ID Numbers

| | | SEQ | SEQ | |
|---|---|---|---|---|
| | | ID | ID | |
| | | NO | NO | |
| Assign- | Internal | (nucleic | (amino | |
| ment | Identification | acid) | acid) | Description |
| NOV1a | CG57067-23 | 1 | 2 | Fc-CC1-CC2-FBD-Fc |
| NOV1b | CG57067-01 | 3 | 4 | AngX (parent sequence) |
| NOV1c | CG57067-02 | 5 | 6 | GeneSeq Acc No: |
| | | | | AAE19826 |
| NOV1d | CG57067-03 | 7 | 8 | AngX-Fc |
| NOV1e | CG57067-04 | 9 | 10 | |
| NOV1f | CG57067-05 | 11 | 12 | Fc-AngX |
| NOV1g | CG57067-06 | 13 | 14 | GeneSeq Acc No: |
| | | | | AAE19826 |
| NOV1h | CG57067-07 | 15 | 16 | GeneSeq Acc No: |
| | | | | AAE19826 |
| NOV1i | CG57067-08 | 17 | 18 | FBD-Fc |
| NOV1j | CG57067-09 | 19 | 20 | Signal p-CC1-CC2-FBG-Fc |
| NOV1k | CG57067-10 | 21 | 22 | Mature CC1-CC2-FBD-Fc |
| NOV1l | CG57067-11 | 23 | 24 | AngX-Fc |
| NOV1m | CG57067-12 | 25 | 26 | CC1-FBD-Fc |
| NOV1n | CG57067-13 | 27 | 28 | CC2-FBD-Fc |
| NOV1o | CG57067-14 | 29 | 30 | FBD(2)-Fc |
| NOV1p | CG57067-15 | 31 | 32 | FBD-Fc |
| NOV1q | CG57067-16 | 33 | 34 | Fc-AngX |
| NOV1r | CG57067-17 | 35 | 36 | Fc-AngX-FBD |
| NOV1s | CG57067-18 | 37 | 38 | Fc-CC1 |
| NOV1t | CG57067-19 | 39 | 40 | Fc-CC2-FBD |
| NOV1ta | CG57067- | 41 | 42 | Fc-CC2-FBD |
| | 19a | | | |
| NOV1u | CG57067-20 | 43 | 44 | Fc-FBD(2) |
| NOV1v | CG57067-21 | 45 | 46 | Fc-FBD |
| NOV1w | CG57067-22 | 47 | 48 | Fc-FBD-Fc |
| NOV1x | CG57067-24 | 49 | 50 | Fc-CC |
| NOV1y | CG57067-25 | 51 | 52 | Fc-FBD |
| NOV1z | CG57067-26 | 53 | 54 | CC |
| NOV1aa | CG57067-27 | 55 | 56 | FBD |
| NOV1ab | CG57067-28 | 57 | 58 | Fc-CC2-FBD(2) |
| NOV1ac | CG57067-29 | 59 | 60 | IFC |
| NOV1ad | CG57067-30 | 61 | 62 | IFC |
| NOV1ae | CG57067-31 | 63 | 64 | CC2-FBD |
| Igk | | 65 | 66 | |
| CC | | 67 | 68 | |
| domain | | | | |
| CC1 | | 69 | 70 | |
| domain | | | | |
| CC2 | | 71 | 72 | |
| domain | | | | |
| Fibrinogen | | 73 | 74 | |
| Binding | | | | |
| Domain | | | | |
| (FBD) | | | | |

The invention further provides novel coiled coil sub-domains sufficient for biological activity when combined with the fibrinogen binding domain (FBD). In a preferred embodiment, the CC subdomains are CC1 polynucleotide SEQ ID NO:69 encoding polypeptide SEQ ID NO:70, and CC2 polynucleotide SEQ ID NO:71, encoding polypeptide SEQ ID NO:72. Another embodiment of the invention is a protein comprising one CC domain chosen from the group consisting of CC1 and CC2 and further comprising a binding domain such as FBD. The present invention further contemplates the use of CC1 or CC2 domains in conjunction with other binding domains such that CC1 or CC2 can form dimer, trimer or oligomerization of the protein construct.

In one embodiment, the present invention provides an isolated polynucleotide selected from the group consisting of:

(a) a polynucleotide comprising the nucleotide sequence SEQ ID NO:73 encoding a Fibrinogen Binding Domain (FBD) and a polynucleotide comprising a fragment of the nucleotide sequence SEQ ID NO:67 encoding a coiled coil (CC) domain;

(b) a polynucleotide comprising the nucleotide sequence SEQ ID NO:73 encoding a Fibrinogen Binding Domain (FBD) and a polynucleotide comprising a the nucleotide sequence SEQ ID NO:69 encoding a coiled coil 1 (CC1) domain;

(c) a polynucleotide comprising the nucleotide sequence SEQ ID NO:73 encoding a Fibrinogen Binding Domain (FBD) and a polynucleotide comprising a the nucleotide sequence SEQ ID NO:71, encoding a coiled coil 2 (CC2) domain;

(d) a polynucleotide encoding a protein comprising the amino acid sequence of SEQ ID NOs: 1, 7, 9, 11, 17, 23, 25, 27, 29, 31, 33, 35, 37, 39, 41, 43, 45, 47, 49, 51, 53, 55, 57, 59, 61, 69, 71; a polynucleotide encoding a protein comprising a fragment of the amino acid sequence SEQ ID NOs: 2, 8, 10, 12, 18, 24, 26, 28, 30, 32, 34, 36, 38, 40, 42, 44, 46, 48, 50, 52, 54, 56, 58, 60, 62, 64, having biological activity;

(e) a polynucleotide capable of hybridizing under stringent conditions to any one of the polynucleotides specified in (a)-(e).

Polynucleotides hybridizing to the polynucleotides of the present invention under stringent conditions and highly stringent conditions are also part of the present invention. As used herein, “highly stringent conditions” include, for example, at least about 0.2 times SSC at 65° C.; and “stringent conditions” include, for example, hybridization in 50% formamide, 1 M NaCl, 1% SDS at 37° C., and a wash in 0.1×SSC (20×SSC=3.0 M NaCl/0.3 M trisodium citrate) at 60-65° C. Preferred high stringency conditions are hybridization in 4×SSC, 5× Denhardt's (5 g Ficoll, 5 g polyvinylpyrrolidone, 5 g bovine serum albumin in 500 ml of water), 0.1 mg/ml boiled salmon sperm DNA, and 25 mM Na phosphate at 65° C., and a wash in 0.1×SSC, 0.1% SDS at 65° C. Allelic variants of the polynucleotides of the present invention are also encompassed by the invention.

In one embodiment, the present invention provides an isolated polynucleotide selected from the group consisting of:

(a) a polynucleotide encoding the amino acid sequence SEQ ID NO:74 Fibrinogen Binding Domain (FBD) and a fragment of an amino acid sequence SEQ ID NO:68 coiled coil (CC) domain;

(b) a polynucleotide encoding the amino acid sequence SEQ ID NO:74 Fibrinogen Binding Domain (FBD) and an amino acid sequence SEQ ID NO SEQ ID NO:70 coiled coil 1 (CC 1) domain;

(c) a polynucleotide encoding the amino acid sequence SEQ ID NO:74 Fibrinogen Binding Domain (FBD) and an amino acid sequence SEQ ID NO:72, coiled coil 2 (CC2) domain;

(d) a polynucleotide encoding an amino acid sequence of SEQ ID NOs: 1, 7, 9, 11, 17, 23, 25, 27, 29, 31, 33, 35, 37, 39, 41, 43, 45, 47, 49, 51, 53, 55, 57, 59, 61, 69, 71; a polynucleotide encoding a protein comprising a fragment of the amino acid sequence SEQ ID NOs: 2, 8, 10, 12, 18, 24, 26, 28, 30, 32, 34, 36, 38, 40, 42, 44, 46, 48, 50, 52, 54, 56, 58, 60, 62, 64, having biological activity;

(e) a polynucleotide capable of hybridizing under stringent conditions to any one of the polynucleotides specified in (a)-(e).

In one embodiment, the present invention provides an isolated polypeptide selected from the group consisting of:

(a) a polypeptide comprising the amino acid sequence SEQ ID NO:74 Fibrinogen Binding Domain (FBD) and an amino acid sequence comprising a fragment of the amino acid sequence SEQ ID NO:68 coiled coil (CC) domain;

(b) a polypeptide comprising the amino acid sequence SEQ ID NO:74 Fibrinogen Binding Domain (FBD) and an amino acid sequence comprising SEQ ID NO: 70, coiled coil 1 (CC1) domain;

(c) a polypeptide comprising the amino acid sequence SEQ ID NO:74 Fibrinogen Binding Domain (FBD) and an amino acid sequence comprising SEQ ID NO:72 coiled coil 2 (CC2) domain;

(d) a polypeptide comprising the amino acid sequence SEQ ID NOs: 2, 8, 10, 12, 18, 24, 26, 28, 30, 32, 34, 36, 38, 40, 42, 44, 46, 48, 50, 52, 54, 56, 58, 60, 62, 64;

(e) a polypeptide comprising a fragment of the amino acid sequence SEQ ID NOs: 2, 8, 10, 12, 18, 24, 26, 28, 30, 32, 34, 36, 38, 40, 42, 44, 46, 48, 50, 52, 54, 56, 58, 60, 62, 64, having biological activity;

(f) a polypeptide encoded by a polynucleotide capable of hybridizing under stringent conditions to any one of the polynucleotides encoding polypeptids specified in (a)-(e).

Conservative Mutations

In addition to the nucleic acid sequences of the invention, the skilled artisan will further appreciate that changes can be introduced by mutation into the nucleotide sequences of of SEQ ID NOs: 1, 7, 9, 11, 17, 23, 25, 27, 29, 31, 33, 35, 37, 39, 41, 43, 45, 47, 49, 51, 53, 55, 57, 59, 61, 69, 71, thereby leading to changes in the amino acid sequences of the encoded protein of the invention, without altering the functional ability of that protein. For example, nucleotide substitutions leading to amino acid substitutions at “non-essential” amino acid residues can be made in the sequence of SEQ ID NOs: 2, 8, 10, 12, 18, 24, 26, 28, 30, 32, 34, 36, 38, 40, 42, 44, 46, 48, 50, 52, 54, 56, 58, 60, 62, 64, 70, 72. A “non-essential” amino acid residue is a residue that can be altered from the sequences of the proteins of the invention without altering their biological activity, whereas an “essential” amino acid residue is required for such biological activity. For example, amino acid residues that are conserved among the proteins of the invention are predicted to be particularly non-amenable to alteration. Amino acids for which conservative substitutions can be made are well-known within the art.

Chimeric and Fusion Proteins

The invention also provides Angioarrestin chimeric or fusion proteins. As used herein, a Angioarrestin “chimeric protein” or “fusion protein” comprises a polypeptide of the invention operatively-linked to a non-angioarrestin polypeptide. An “Angioarrestin polypeptide” refers to a polypeptide having an amino acid sequence SEQ ID NOs: 2, 8, 10, 12, 18, 24, 26, 28, 30, 32, 34, 36, 38, 40, 42, 44, 46, 48, 50, 52, 54, 56, 58, 60, 62, 64, whereas a “non-Angioarrestin polypeptide” refers to a polypeptide having an amino acid sequence corresponding to a protein that is not substantially homologous to the Angioarrestin protein, e.g., a protein that is different from the Angioarrestin protein and that may be derived from the same or a different organism. Within an Angioarrestin fusion protein the Angioarrestin polypeptide can correspond to all or a portion of an Angioarrestin protein. In one embodiment, an Angioarrestin fusion protein comprises at least one biologically-active portion of an Angioarrestin protein. In another embodiment, an Angioarrestin fusion protein comprises at least two biologically-active portions of an Angioarrestin protein. In yet another embodiment, an Angioarrestin fusion protein comprises at least three biologically-active portions of an Angioarrestin protein. Within the fusion protein, the term “operatively-linked” is intended to indicate that the Angioarrestin polypeptide and the non-Angioarrestin polypeptide are fused in-frame with one another. The non-Angioarrestin polypeptide can be fused to the N-terminus or C-terminus of the Angioarrestin polypeptide.

In one embodiment, the fusion protein is a GST-ANGIOARRESTIN fusion protein in which the ANGIOARRESTIN sequences are fused to the C-terminus of the GST (glutathione S-transferase) sequences. Such fusion proteins can facilitate the purification of recombinant ANGIOARRESTIN polypeptides.

In another embodiment, the fusion protein is a ANGIOARRESTIN protein containing a heterologous signal sequence at its N-terminus. In certain host cells (e.g., mammalian host cells), expression and/or secretion of ANGIOARRESTIN can be increased through use of a heterologous signal sequence.

In yet another embodiment, the fusion protein is a ANGIOARRESTIN-immunoglobulin fusion protein in which the ANGIOARRESTIN sequences are fused to sequences derived from a member of the immunoglobulin protein family. The ANGIOARRESTIN-immunoglobulin fusion proteins of the invention can be incorporated into pharmaceutical compositions and administered to a subject to inhibit an interaction between a ANGIOARRESTIN ligand and a ANGIOARRESTIN protein on the surface of a cell, to thereby suppress ANGIOARRESTIN-mediated signal transduction in vivo. The ANGIOARRESTIN-immunoglobulin fusion proteins can be used to affect the bioavailability of a ANGIOARRESTIN cognate ligand. Inhibition of the ANGIOARRESTIN ligand/ANGIOARRESTIN interaction may be useful therapeutically for both the treatment of proliferative and differentiative disorders, as well as modulating (e.g. promoting or inhibiting) cell survival. Moreover, the ANGIOARRESTIN-immunoglobulin fusion proteins of the invention can be used as immunogens to produce anti-ANGIOARRESTIN antibodies in a subject, to purify ANGIOARRESTIN ligands, and in screening assays to identify molecules that inhibit the interaction of ANGIOARRESTIN with a ANGIOARRESTIN ligand.

An ANGIOARRESTIN chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) Currrent Protocols in Molecular Biology, John Wiley & Sons, 1992). Moreover, many expression vectors are commercially available that already encode a fusion moiety (e.g., a GST polypeptide). An ANGIOARRESTIN-encoding nucleic acid can be cloned into such an expression vector such that the fusion moiety is linked in-frame to the ANGIOARRESTIN protein.

ANGIOARRESTIN Recombinant Expression Vectors and Host Cells

Another aspect of the invention pertains to vectors, preferably expression vectors, containing a nucleic acid encoding an ANGIOARRESTIN protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, Gene Expression Technology: Methods in Enzymology 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., ANGIOARRESTIN proteins, mutant forms of ANGIOARRESTIN proteins, fusion proteins, etc.).

The recombinant expression vectors of the invention can be designed for expression of ANGIOARRESTIN proteins in prokaryotic or eukaryotic cells. For example, ANGIOARRESTIN proteins can be expressed in bacterial cells such as Escherichia coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., Gene Expression Technology: Methods in Enzymology 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, Gene Expression Technology: Methods in Enzymology 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the ANGIOARRESTIN expression vector is a yeast expression vector. Examples of vectors for expression in yeast Saccharomyces cerivisae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (Invitrogen Corp, San Diego, Calif.).

Alternatively, ANGIOARRESTIN can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., Molecular Cloning: A Laboratory Manual. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to ANGIOARRESTIN mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, ANGIOARRESTIN protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding ANGIOARRESTIN or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) ANGIOARRESTIN protein. Accordingly, the invention further provides methods for producing ANGIOARRESTIN protein using the host cells of the invention. In one embodiment, the method comprises culturing the host cell of invention (into which a recombinant expression vector encoding ANGIOARRESTIN protein has been introduced) in a suitable medium such that ANGIOARRESTIN protein is produced. In another embodiment, the method further comprises isolating ANGIOARRESTIN protein from the medium or the host cell.

To isolate parent Angioarrestin nucleic acid molecule, messenger RNA (mRNA) was purified from total cellular RNA isolated from various human organs which were commercially-available from Clontech (e.g., Fetal brain, heart, kidney, fetal liver, liver, lung, skeletal muscle, pancreas and placenta) utilizing an Oligotex™ cDNA synthesis kit (QIAGEN, Inc.; Chatsworth, Calif.). The first-strand of the cDNA was prepared from 1.0 μg of poly(A)⁺ RNA with 200 pmols of oligo(dT)25V (wherein V=A, C or G) using 400 units of Superscript II reverse transcriptase (BRL; Grand Island N.Y.). Following the addition of 10 units of E. coli DNA ligase, 40 units of E. coli DNA polymerase, and 3.5 units of E. coli RNase H (all supplied by BRL; Grand Island, N.Y.), second-strand synthesis was performed at 16° C. for 2 hours. Five units of T₄ DNA polymerase was then added, and incubation was continued for an additional 5 minutes at 16° C. The reaction was then treated with 5 units of arctic shrimp alkaline phosphatase (U.S. Biochemicals; Cleveland Ohio) at 37° C. for 30 minutes, and the cDNA was purified by standard phenol/chloroform (50:50 v/v) extraction. The yield of cDNA was estimated using fluorometry with the Picogreen™ Labeling System (Molecular Probes; Eugene, Oreg.).

Following synthesis, the double-stranded cDNA was digested with various restriction enzymes, ligated to linkers compatible with the over-hanging termini generated by the restriction digestion. The restriction fragments were amplified utilizing 30 cycles of polymerase chain reaction (PCR) by the addition of the following reagents: 2 μl 10 mM dNTP; 5 μl 10× TB buffer (500 mM Tris, 160 mM (NH4)₂SO₄,; 20 mM MgCl₂, pH 9.15); 0.25 μl Klentaq (Clontech Advantage): PFU (Stratagene; La Jolla Calif.) in a 16:1 v/v ratio; 32.75 μl ddH2O. The amplification products were then ligated into the TA™ cloning vector (Invitrogen). Individual clones were subjected to dye-primer, double-stranded DNA sequencing utilizing PCR products which were derived from amplification using vector-specific primers, which flanked the insertion, site as templates. Sequencing was performed using a standard chemistry methdology on ABI Model 377 sequencers (Molecular Dynamics).

The isolated polynucleotide of the invention may be operably linked to an expression control sequence such as the pMT2 or pED expression vectors disclosed in Kaufman et al., Nucleic Acids Res. 19, 4485-4490 (1991), in order to produce the protein recombinantly. Many suitable expression control sequences are known in the art. General methods of expressing recombinant proteins are also known and are exemplified in R. Kaufman, Methods in Enzymology 185, 537-566 (1990). As defined herein “operably linked” means that the isolated polynucleotide of the invention and an expression control sequence are situated within a vector or cell in such a way that the protein is expressed by a host cell which has been transformed (transfected) with the ligated polynucleotide/expression control sequence.

A number of types of cells may act as suitable host cells for expression of the protein. Mammalian host cells include, for example, monkey COS cells, Chinese Hamster Ovary (CHO) cells, human kidney 293 cells, human epidermal A431 cells, human Colo205 ells, 3T3 cells, CV-1 cells, other transformed primate cell lines, normal diploid cells, cell strains derived from in vitro culture of primary tissue, primary explants, HeLa cells, mouse L cells, BHK, HL-60, U937, HaK or Jurkat cells.

Alternatively, the protein can be produced in lower eukaryotes such as yeast, or in prokaryotes such as bacteria. Yeast strains can include, e.g., Saccharomyces cerevisiae, Schizosaccharomyces pombe, Kluyveromyces strains, Candida spp., or any other yeast strain capable of expressing heterologous proteins. Potentially suitable bacterial strains include Escherichia coli, Bacillus subtilis, Salmonella typhimurium, or any bacterial strain capable of expressing heterologous proteins. If the protein is made in yeast or bacteria, it may be necessary to modify the protein produced therein, for example by phosphorylation or glycosylation of the appropriate sites, in order to obtain the functional protein. Such covalent attachments may be accomplished using known chemical or enzymatic methods.

The protein may also be produced by operably linking the isolated polynucleotide of the invention to suitable control sequences in one or more insect expression vectors, and employing an insect expression system. Materials and methods for baculovirus/insect cell expression systems are commercially available in kit form from, e.g., Invitrogen, San Diego, Calif., U.S.A. (the MaxBac.R™. kit), and such methods are well known in the art, as described in Summers and Smith, Texas Agricultural Experiment Station Bulletin No. 1555 (1987), incorporated herein by reference. As used herein, an insect cell capable of expressing a polynucleotide of the present invention is “transformed.”

Biological Activity of Protein Fragments

Fragments of the proteins of the present invention which are capable of exhibiting biological activity are also encompassed by the present invention. Fragments of the protein may be in linear form or they may be cyclized using known methods, for example, as described in H. U. Saragovi, et al., Bio/Technology 10, 773-778 (1992) and in R. S. McDowell et al., J. Amer. Chem. Soc. 114, 9245-9253 (1992). Such fragments may be fused to carrier molecules such as immunoglobulins for many purposes, including increasing the valency of protein binding sites, increasing the stability of the protein, effecting in vivo half lives of proteins, etc.. For example, fragments of the protein may be fused optionally through “linker” sequences to for example, the Fc portion of an immunoglobulin i.e. the Fc portion of an IgG molecule. Other immunoglobulin isotypes may also be used to generate such fusions. For example, a protein-IgM fusion would generate a decavalent form of the protein of the invention.

Protein Purification

The protein of the invention may be prepared by culturing transformed host cells under culture conditions suitable to express the recombinant protein. The resulting expressed protein may then be purified from such culture (i.e., from culture medium or cell extracts) using known purification processes, such as gel filtration and ion exchange chromatography. The purification of the protein may also include an affinity column containing agents which will bind to the protein; one or more column steps over such affinity resins as concanavalin A-agarose, heparin-toyopearl R™ or Cibacrom blue 3GA Sepharose R™; one or more steps involving hydrophobic interaction chromatography using such resins as phenyl ether, butyl ether, or propyl ether; or immunoaffinity chromatography.

Alternatively, the protein of the invention may also be expressed in a form which will facilitate purification. For example, it may be expressed as a fusion protein, such as those of maltose binding protein (MBP), glutathione-S-transferase (GST) or thioredoxin (TRX). Kits for expression and purification of such fusion proteins are commercially available from New England BioLab (Beverly, Mass.), Pharmacia (Piscataway, N.J.) and InVitrogen, respectively. The protein can also be tagged with an epitope and subsequently purified by using a specific antibody directed to such epitope. One such epitope (“Flag”) is commercially available from Kodak (New Haven, Conn.).

Finally, one or more reverse-phase high performance liquid chromatography (RP-HPLC) steps employing hydrophobic RP-HPLC media, e.g., silica gel having pendant methyl or other aliphatic groups, can be employed to further purify the protein. Some or all of the foregoing purification steps, in various combinations, can also be employed to provide a substantially homogeneous isolated recombinant protein. The protein thus purified is substantially free of other mammalian proteins and is defined in accordance with the present invention as an “isolated protein.”

The protein of the invention may also be expressed as a product of transgenic animals, e.g., as a component of the milk of transgenic cows, goats, pigs, or sheep which are characterized by somatic or germ cells containing a nucleotide sequence encoding the protein.

The protein may also be produced by known conventional chemical synthesis. Methods for constructing the proteins of the present invention by synthetic means are known to those skilled in the art. The synthetically-constructed protein sequences, by virtue of sharing primary, secondary or tertiary structural and/or conformational characteristics with proteins may possess biological properties in common therewith, including protein activity. Thus, they may be employed as biologically active or immunological substitutes for natural, purified proteins in screening of therapeutic compounds and in immunological processes for the development of antibodies.

Modified Proteins

The proteins provided herein also include proteins characterized by amino acid sequences similar to those of purified proteins but into which modification are naturally provided or deliberately engineered. For example, modifications in the peptide or DNA sequences can be made by those skilled in the art using known techniques. Modifications of interest in the protein sequences may include the replacement, insertion or deletion of a selected amino acid residue in the coding sequence. For example, one or more of the cysteine residues may be deleted or replaced with another amino acid to alter the conformation of the molecule. Mutagenic techniques for such replacement, insertion or deletion are well known to those skilled in the art (see, e.g., U.S. Pat. No. 4,518,584, incorporated by reference).

Other fragments and derivatives of the sequences of proteins which would be expected to retain protein activity in whole or in part and may thus be useful for screening or other immunological methodologies may also be easily made by those skilled in the art given the disclosures herein. Such modifications are believed to be encompassed by the present invention.

Uses and Biological Activity

The polynucleotides of the present invention and the proteins encoded thereby are expected to exhibit one or more of the uses or biological activities identified below. Uses or activities described for proteins of the present invention may be provided by administration or use of such proteins or by administration or use of polynucleotides encoding such proteins (such as, for example, in gene therapies or vectors suitable for introduction of DNA).

The biological activity of the proteins of this invention can be assayed by any suitable method known in the art. The angiogenic/antiangiogenic potential can be characterized in angiogenesis assays in vivo such as the chick chorionic allantoic membrane (CAM) assay or different cornea micropocket assays (Klagsbrun & Folkman, 1990, In: Sporn & Roberts (eds). Peptide growth factors and their receptors II, pp. 549-574). An a in vivo angiogenesis assay is described in, eg., U.S. Pat. No. 5,382,514), and a mouse model of hindlimb ischemia was described by Couffinhal et al., 1998, Am. J. Pathol. 152:1667-1679. Direct effects of angiogenic molecules on vascular wall cells can be assayed in in vitro assays. These assays facilitate the study of endothelial functions that are essential for new blood vessel formation. Most in vitro models of angiogenesis use extracellular matrix substrata containing growth-regulatory molecules (Vukicevic et al., 1992, Exp. Cell. Res. 202:1-8). Furthermore, cell culture assays utilized to test angiopoietin for formation of capillary sprouts may be used (see, eg. Koblizek et al., 1998, Curr. Biol. 8:529-532). Most assays require exogenous stimuli such as phorbol esters or angiogenic molecules to induce the formation of endothelial cords and tubes. Assays for angiogenic/antiangiogenic activity include methods for inhibition of angiogenesis (see, for example, but not limited to, U.S. Pat. Nos. 5,733,876, 5,639,725, 5,712,291, 5,698,586, 5,753,230, 5,733,876, 5,766,591, 5,434,185, 5,721,226, 5,629,340, 5,593,990, 5,629,327, 5,744,492, 5,646,136, 5,610,166, 5,574,026, 5,567,693, 5,563,130).

Angiogenic Stimulation/Inhibition Activity

A protein of the present invention exhibits anti-angiogenic or cell differentiation (either inducing or inhibiting) activity. The activity of a protein of the present invention is evidenced by any one of a number of routine factor dependent cell proliferation assays. Methods of assaying an Angioarrestion molecule for activity as a therapeutic as well as methods of screening modulators (i.e., inhibitors, agonists and antagonists) are also contemplated.

Pharmaceutical Compositions

The ANGIOARRESTIN nucleic acid molecules, ANGIOARRESTIN proteins, and anti-ANGIOARRESTIN antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a ANGIOARRESTIN protein or anti-ANGIOARRESTIN antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Administration and Dosing

A protein of the present invention (from whatever source derived, including without limitation from recombinant and non-recombinant sources) may be used in a pharmaceutical composition when combined with a pharmaceutically acceptable carrier. Such a composition may also contain (in addition to protein and a carrier) diluents, fillers, salts, buffers, stabilizers, solubilizers, and other materials well known in the art. The term “pharmaceutically acceptable” means a non-toxic material that does not interfere with the effectiveness of the biological activity of the active ingredient(s). The characteristics of the carrier will depend on the route of administration. The pharmaceutical composition may further contain other agents which either enhance the activity of the protein or compliment its activity or use in treatment. Such additional factors and/or agents may be included in the pharmaceutical composition to produce a synergistic effect with protein of the invention, or to minimize side effects. Conversely, protein of the present invention may be included in formulations of the particular Angioarrestin to minimize side effects of the agent.

Administration of protein of the present invention used in the pharmaceutical composition or to practice the method of the present invention can be carried out in a variety of conventional ways, such as oral ingestion, inhalation, or cutaneous, subcutaneous, or intravenous injection. When a therapeutically effective amount of protein of the present invention is administered by intravenous, cutaneous or subcutaneous injection, protein of the present invention will be in the form of a pyrogen-free, parenterally-acceptable aqueous solution. The preparation of such parenterally acceptable protein solutions, having due regard to pH, isotonicity, stability, and the like, is within the skill in the art. A preferred pharmaceutical composition for intravenous, cutaneous, or subcutaneous injection should contain, in addition to protein of the present invention, an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition of the present invention may also contain stabilizers, preservatives, buffers, antioxidants, or other additives known to those of skill in the art.

A protein of the present invention may be active in monomer, dimmer, tetromer or multimers (hetero or homo) complexes with itself or other proteins. As a result, pharmaceutical compositions of the invention may comprise a protein of the invention in such forms.

As used herein, the term “therapeutically effective amount” means the total amount of each active component of the pharmaceutical composition or method that is sufficient to show a meaningful patient benefit, i.e., treatment, healing, prevention or amelioration of the relevant medical condition, or an increase in rate of treatment, healing, prevention or amelioration of such conditions. When applied to an individual active ingredient, administered alone, the term refers to that ingredient alone. When applied to a combination, the term refers to combined amounts of the active ingredients that result in the therapeutic effect, whether administered in combination, serially or simultaneously.

In practicing the method of treatment or use of the present invention, a therapeutically effective amount of protein of the present invention is administered to a mammal having a condition to be treated. Protein of the present invention may be administered in accordance with the method of the invention either alone or in combination with other therapies. When co-administered with one or more other therapies, protein of the present invention may be administered either simultaneously or sequentially. If administered sequentially, the attending physician will decide on the appropriate sequence of administering protein of the present invention in combination therapies.

The amount of protein of the present invention in the pharmaceutical composition of the present invention will depend upon the nature and severity of the condition being treated, and on the nature of prior treatments which the patient has undergone. Ultimately, the attending physician will decide the amount of protein of the present invention with which to treat each individual patient. Initially, the attending physician will administer low doses of protein of the present invention and observe the patient's response. Larger doses of protein of the present invention may be administered until the optimal therapeutic effect is obtained for the patient, and at that point the dosage is not increased further.

Accordingly, in one aspect, the invention includes a method for inhibiting cell proliferation by providing the cell (e.g., ex vivo, in vitro, or in vivo) with an amount of the protein of the invention sufficient to inhibit proliferation of the cell. The cell can be, e.g., en endothelial cell such as a human vascular endothelial cell.

The invention also provides a method for inhibiting the growth of a tumor in a subject by administering to the subject an Angioarrestin polypeptide in an amount sufficient to inhibit the growth of the tumor.

The subject is preferably a mammal, e.g., a human, or non-human primate, dog, cat, horse, cow, or pig.

Also within the invention is a method for inhibiting the growth of a tumor in a subject by administering to the subject an amount of an Angioarrestin nucleic acid in an amount sufficient to inhibit the growth of the tumor. Such Angioarrestin nucleic acid is administered in conjunction with operable components to cause expression and tumor cell exposure to Angioarrestin protein.

Also within the invention is a method for inhibiting tumor metastasis in a subject by administering to the subject an amount of an Angioarrestin polypeptide in an amount sufficient to inhibit metastasis of the tumor. Alternatively, a nucleic acid encoding an Angioarrestin polypeptide can be administered in an amount sufficient to inhibit metastasis of the tumor. The tumor can be, a fibrosarcoma or a carcinoma.

Antibodies

A protein of the invention may also be used to immunize animals to obtain polyclonal and monoclonal antibodies which specifically react with the protein. Such antibodies may be obtained using either the entire protein or fragments thereof as an immunogen. The peptide immunogens additionally may contain a cysteine residue at the carboxyl terminus, and are conjugated to a hapten such as keyhole limpet hemocyanin (KLH). Methods for synthesizing such peptides are known in the art, for example, as in R. P. Merrifield, J. Amer. Chem. Soc. 85, 2149-2154 (1963); J. L. Krstenansky, et al., FEBS Lett. 211, 10 (1987). Monoclonal antibodies binding to the protein of the invention may be useful diagnostic agents for the immunodetection of the protein. Neutralizing monoclonal antibodies binding to the protein may also be useful therapeutics for both conditions associated with the protein and also in the treatment of some forms of cancer where abnormal expression of the protein is involved. In the case of cancerous cells or leukemic cells, neutralizing monoclonal antibodies against the protein may be useful in detecting and preventing the metastatic spread of the cancerous cells, which may be mediated by the protein.

Gene Therapy

Polynucleotides of the present invention can also be used for gene therapy. Such polynucleotides can be introduced either in vivo or ex vivo into cells then administered to a mammalian subject. Polynucleotides of the invention may also be administered by other known methods for introduction of nucleic acid into a cell or organism (including, without limitation, in the form of viral vectors or naked DNA).

The invention will be further illustrated in the following examples, which do not limit the scope of the claims.

### EXAMPLES

### Example 1

TABLE 2
NOV1 Sequence Analysis

| | |
|---|---|
| SEQ ID NO:1 | 2652 bp |

| | |
|---|---|
| NOV 1a, | GACAAAACTCACACATGCCCACCGTGCCCAGCACCTGAACTCCTGGGGGGACCGTCAG |
| CG57067-23 | |
| DNA Sequence | TCTTCCTCTTCCCCCCAAAACCCAAGGACACCCTCATGATCTCCCGGACCCCTGAGGT |
| | CACATGCGTGGTGGTGGACGTGAGCCACGAAGACCCTGAGGTCAAGTTCAACTGGTAC |
| | GTGGACGGCGTGGAGGTGCATAATGCCAAGACAAAGCCGCGGGAGGAGCAGTACAACA |
| | GCACGTACCGTGTGGTCAGCGTCCTCACCGTCCTGCACCAGGACTGGCTGAATGGCAA |
| | GGAGTACAAGTGCAAGGTCTCCAACAAAGCCCTCCCAGCCCCCATCGAGAAAACCATC |
| | TCCAAAGCCAAAGGGCAGCCCCGAGAACCACAGGTGTACACCCTGCCCCCATCCCGGG |
| | ATGAGCTGACCAAGAACCAGGTCAGCCTGACCTGCCTGGTCAAAGGCTTCTATCCCAG |
| | CGACATCGCCGTGGAGTGGGAGAGCAATGGGCAGCCGGAGAACAACTACAAGACCACG |
| | CCTCCCGTGCTGGACTCCGACGGCTCCTTCTTCCTCTACAGCAAGCTCACCGTGGACA |
| | AGAGCAGGTGGCAGCAGGGGAACGTCTTCTCATCCTCCGTGATGCATGAGGCTCTGCA |
| | CAACCACTACACGCAGAAGAGCCTCTCCCTGTCTCCCGGTAAAGGCGGCGGCGGCGGC |
| | GGCGGCGGCATGATCACCAGGATGGACCTTGAAAACCTGAAGGATGTGCTCTCCAGGC |
| | AGAAGCGGGAGATAGATGTTCTGCAACTGGTGGTGGATGTAGATGGAAACATTGTGAA |
| | TGAGGTAAAGCTGCTGAGAAAGGAAAGCCGTAACATGAACTCTCGTGTTACTCAACTC |
| | TATATGCAATTATTACATGAGATTATCCGTAAGAGGGATAATTCACTTGAACTTTCCC |
| | AACTGGAAAACAAAATCCTCAATGTCACCACAGAAATGTTGAAGATGGCAACAAGATA |
| | CAGGGAACTAGAGGTGAAATACGCTTCCTTGACTGATCTTGTCAATAACCAATCTGTG |
| | ATGATCACTTTGTTGGAAGAACAGTGCTTGAGGATATTTTCCCGACAAGACACCCATG |
| | TGTCTCCCCCACTTGTCCAGGTGGTGCCACAACATATTCCTAACAGCCAACAGTATAC |
| | TCCTGGTCTGCTGGGAGGTAACGAGATTCAGAGGGATCCAGGTTATCCCAGAGATTTA |
| | ATGCCACCACCTGATCTGGCAACTTCTCCCACCAAAAGCCCTTTCAAGATACCACCGG |
| | TAACTTTCATCAATGAAGGACCATTCAAAGACTGTCAGCAAGCAAAAGAAGCTGGGCA |
| | TTCGGTCAGTGGGATTTATATGATTAAACCTGAAAACAGCAATGGACCAATGCAGTTA |
| | TGGTGTGAAAACAGTTTGGACCCTGGGGGTTGGACTGTTATTCAGAAAAGAACAGACG |
| | GCTCTGTCAACTTCTTCAGAAATTGGGAAAATTATAAGAAAGGGTTTGGAAACATTGA |
| | CGGAGAATACTGGCTTGGACTGGAAAATATCTATATGCTTAGCAATCAAGATAATTAC |
| | AAGTTATTGATTGAATTAGAAGACTGGAGTGATAAAAAAGTCTATGCAGAATACAGCA |
| | GCTTTCGTCTGGAACCTGAAAGTGAATTCTATAGACTGCGCCTGGGAACTTACCAGGG |
| | AAATGCAGGGGATTCTATGATGTGGCATAATGGTAAACAATTCACCACACTGGACAGA |
| | GATAAAGATATGTATGCAGGAAACTGCGCCCACTTTCATAAAGGAGGCTGGTGGTACA |
| | ATGCCTGTGCACATTCTAACCTAAATGGAGTATGGTACAGAGGAGGCCATTACAGAAG |
| | CAAGCACCAAGATGGAATTTTCTGGGCCGAATACAGAGGCGGGTCATACTCCTTAAGA |
| | GCAGTTCAGATGATGATCAAGCCTATTGACGGCGGCGGCGGCGGCGGCGGCGGCGACA |
| | AAACTCACACATGCCCACCGTGCCCAGCACCTGAACTCCTGGGGGGACCGTCAGTCTT |
| | CCTCTTCCCCCCAAAACCCAAGGACACCCTCATGATCTCCCGGACCCCTGAGGTCACA |
| | TGCGTGGTGGTGGACGTGAGCCACGAAGACCCTGAGGTCAAGTTCAACTGGTACGTGG |
| | ACGCCGTGGAGGTGCATAATGCCAAGACAAAGCCGCGGGAGGAGCAGTACAACAGCAC |
| | GTACCGTGTGGTCAGCGTCCTCACCGTCCTGCACCAGGACTGGCTGAATGGCAAGGAG |
| | TACAAGTGCAAGGTCTCCAACAAAGCCCTCCCAGCCCCCATCGAGAAAACCATCTCCA |
| | AAGCCAAAGGGCAGCCCCGAGAACCACAGGTGTACACCCTGCCCCCATCCCGGGATGA |
| | GCTGACCAAGAACCAGGTCAGCCTGACCTGCCTGGTCAAAGGCTTCTATCCCAGCGAC |
| | ATCGCCGTGGAGTGGGAGAGCAATGGGCAGCCGGAGAACAACTACAAGACCACGCCTC |
| | CCGTGCTGGACTCCGACGGCTCCTTCTTCCTCTACAGCAAGCTCACCGTGGACAAGAG |
| | CAGGTGGCAGCAGGGGAACGTCTTCTCATGCTCCGTGATGCATGAGGCTCTGCACAAC |
| | CACTACACGCAGAAGAGCCTCTCCCTGTCTCCGGGTAAATGA |

| | |
|---|---|
| ORF Start: at 1 | ORF Stop: IGA at 2650 |

| | | |
|---|---|---|
| SEQ ID NO: 2 | 883 aa | MW at 99842.3kD |

| | |
|---|---|
| NOV1a, | DKTHTCPPCPAPELLGGPSVFLFPPKPKDTLMISRTPEVTCVVVDVSHEDPEVKFNWY |
| CG57067-23 | |
| Protein | VDGVEVHNAKTKPREEQYNSTYRVVSVLTVLHQDWLNGKEYKCKVSNKALPAPIEKTI |
| Sequence | |
| | SKAKGQPREPQVYTLPPSRDELTKNQVSLTCLVKCFYPSDIAVEWESNCQPENNYKTT |
| | PPVLDSDGSFFLYSKLTVDKSRWQQGNVFSCSVMHEALHNHYTQKSLSLSPGKGGGGG |
| | GGGMITRMDLENLKDVLSRQKREIDVLQLVVDVDGNIVNEVKLLRKESRNMNSRVTQL |
| | YMQLLHEIIRKRDNSLELSQLENKILNVTTEMLKMATRYRELEVKYASLTDLVNNQSV |
| | MITLLEEQCLRIFSRQDTHVSPPLVQVVPQHIPNSQQYTPGLLGGNEIQRDFGYPRDL |
| | MPPPDLATSPTKSPFKIPPVTFINEGPFKDCQQAKEAGHSVSGIYMIKPENSNGPMQL |
| | WCENSLDPGGWTVIQKRTDGSVNFFRNWENYKKGFGNIDGEYWLGLENIYMLSNQDNY |
| | KLLIELEDWSDKKVYAEYSSFRLEPESEFYRLRLGTYQGNAGDSMMWHNGKQFTTLDR |
| | DKDMYAGNCAHFHKGGWWYNACAHSNLNGVWYRGGHYRSKHQDGIFWAEYRGGSYSLR |
| | AVQMMIKPIDGGGGGGGGDKTHTCPPCPAPELLGGPSVFLFPPKPKDTLMISRTPSVT |
| | CVVVDVSHEDPEVKFNWYVDGVEVHNAKTKPREEQYNSTYRVVSVLTVLHQDWLNGKE |
| | YKCKVSNKALPAPIEKTISKAKGQPREPQVYTLPPSRDELTKNQVSLTCLVKGFYPSD |
| | IAVEWESNGQPENNYKTTPPVLDSDGSFFLYSKLTVDKSRWQQGNVFSCSVMHEALHN |
| | HYTQKSLSLSPGK |

| | |
|---|---|
| SEQ ID NO: 3 | 2354 bp |

| | |
|---|---|
| NOV1b, | ATCTGGGTCAGCTGCAGCTGGTTACTGCATTTCTCCATGTGGCAGACAGAGCAAAGCC |
| CG57067-01 | |
| DNA Sequence | ACAACGCTTTCTCTGCTGGATTAAAGACGGCCCACAQACCAGAACTTCCACTATACTA |
| | CTTAAAATTACATAGGTGGCTTGTCAAATTCAATTGATTAGTATTGTAAAAGGAAAAA |
| | GAAGTTCCTTCTTACAGCTTGGATTCAACGGTCCAAAACAAAAATGCAGCTGCCATTA |
| | AAGTCACAGATGAACAAACTTCTACACTGATTTTTAAAATCAAGAATAAGGGCAGCAA |
| | GTTTCTGGATTCACTGAATCAACAGACACAAAAAGACATCATTTTACAACCTCATTTC |
| | AAAATGAAGACTTTTACCTGGACCCTAGGTGTGCTATTCTTCCTACTAGTGGACACTG |
| | GACATTGCAGAGGTGGACAATTCAAAATTAAAAAAATAAACCAGAGAAGATACCCTCG |
| | TGCCACAGATGGTAAAGAGGAAGCAAAGAAATGTGCATACACATTCCTGGTACCTGAC |
| | CAAAGAATAACAGGGCCAATCTGTGTCAACACCAAGGGGCAAGATGCAAGTACCATTA |
| | AAGACATGATCACCAGGATGGACCTTGAAAACCTGAAGGATGTGCTCTCCAGGCAGAA |
| | GCGGGAGATAGATGTTCTGCAACTGGTGGTGGATGTAGATGGAAACATTGTGAATGAG |
| | GTAAAGCTGCTGAGAAAGGAAAGCCGTAACATGAACTCTCGTGTTACTCAACTCTATA |
| | TGCAATTATTACATGAGATTATCCGTAAGACGGATAATTCACTTGAACTTTCCCAACT |
| | GGAAAACAAAATCCTCAATGTCACCACACAAATGTTGAAGATGGCAACAAGATACAGG |
| | GAACTAGAGGTGAAATACGCTTCCTTGACTGATCTTGTCAATAACCAATCTGTGATGA |
| | TCACTTTGTTGGAAGAACAGTGCTTGAGGATATTTTCCCGACAAGACACCCATGTGTC |
| | TCCCCCACTTGTCCAGGTGGTGCCACAACATATTCCTAACAGCCAACAGTATACTCCT |
| | GGTCTGCTGGGAGGTAACGAGATTCAGAGGGATCCAGGTTATCCCAGAGATTTAATGC |
| | CACCACCTGATCTGGCAACTTCTCCCACCAAAAGCCCTTTCAACATACCACCGGTAAC |
| | TTTCATCAATGAAGGACCATTCAAAGACTGTCAGCAAGCAAAAGAAGCTGGGCATTCG |
| | GTCAGTGGGATTTATATGATTAAACCTGAAAACAGCAATGGACCAATGCAGTTATGGT |
| | GTGAAAACAGTTTGGACCCTGGGGGTTGGACTGTTATTCAGAAAAGAACAGACGGCTC |
| | TGTCAACTTCTTCAGAAATTGGGAAAATTATAAGAAAGGGTTTGGAAACATTGACGGA |
| | GAATACTGGCTTGCACTGGAAAATATCTATATGCTTAGCAATCAAGATAATTACAAGT |
| | TATTGATTGAATTAGAAGACTGGAGTGATAAAAAAGTCTATGCAGAATACACCAGCTT |
| | TCGTCTGGAACCTGAAAGTGAATTCTATAGACTGCGCCTGGGAACTTACCAGGGAAAT |
| | GCAGGGGATTCTATGATGTGGCATAATGGTAAACAATTCACCACACTGGACAGAGATA |
| | AAGATATGTATGCAGGAAACTGCGCCCACTTTCATAAAGGAGGCTGGTGGTACAATGC |
| | CTGTGCACATTCTAGCCTAAATGGAGTATGGTACAGAGGAGGCCATTACAGAAGCAAG |
| | CACCAAGATGGAATTTTCTGGGCCGAATACAGAGGCGGGTCATACTCCTTAAGAGCAG |
| | TTCAGATGATGATCAAGCCTATTGACTGAAGAQAGACACTCGCCAATTTAAATGACAC |
| | AGAACTTTGTACTTTTCAGCTCTTAAAAATGTAAATGTTACATGTATATTACTTCGCA |
| | CAATTTATTTCTACACATAAAGTTTTTAAAATGAATTTTACCGTAACTATAAAAGGGA |
| | ACCTATAAATGTAGTTTCATCTGTCGTCAATTACTGCAGAAAATTATGTGTATCCACA |
| | ACCTAGTTATTTTAAAAATTATGTTGACTAAATACAAAGTTTGGTTTCTAAAATGTAA |
| | ATATTTGCCACAATGTAAAGCAAATCTTAGCTATATTTTAAATCATAAATAACATGTT |
| | CAAGATACTTAACAATTTATTTAAAATCTAAGATTGCTCTAACGTCTAGTGAAAAAAA |
| | TATTTTTAAAATTTCAGCCAAATGATGCATTTTATTTATAAAAATACAGACAGAAAAT |
| | TAGGGAGAAACCTCTAGTTTTGCCAATAGAAAATGCTTCTTCCATTGAATAAAAGTTA |
| | TTTCAAATCCAAAAAAAAAAAAAAAAAAAAAAAA |

| | |
|---|---|
| ORF Start: ATG at 352 | ORF Stop: TGA at 1825 |

| | | |
|---|---|---|
| SEQ ID NO: 4 | 491 aa | MW at 56678.1 kD |

| | |
|---|---|
| NOV1b, | MKTFTWTLGVLFFLLVDTGHCRGGQFKIKKINQRRYPRATDGKEEAKKCAYTFLVPDQ |
| CG57067-01 | |
| Protein | RITGPICVNTKGQDASTIKDMITRMDLENLKDVLSRQKREIDVLQLVVDVDGNIVNEV |
| Sequence | |
| | KLLRKESRNMNSRVTQLYMQLLHEIIRKRDNSLELSQLENKILNVTTEMLKMATRYRE |
| | LEVKYASLTDLVNNQSVMITLLEEQCLRIFSRQDTHVSPPLVQVVPQHIPNSQQYTPG |
| | LLGGNEIQRDPGYPRDLMPPPDLATSPTKSPFKIPPVTFINEGPFKDCQQAKEAGHSV |
| | SGIYMIKPENSNGPMQLWCENSLDPGGWTVIQKRTDGSVNFFRNWENYKKGFGNIDGE |
| | YWLGLENIYMLSNQDNYKLLIELEDWSDKKVYAEYSSFRLEPESEFYRLRLGTYQGNA |
| | GDSMMWHNGKQFTTLDRDKDMYAGNCAHFHKGGWWYNACAHSSLNGVWYRGGHYRSKH |
| | QDGIFWAEYRGGSYSLRAVQMMIKPID |

| | |
|---|---|
| SEQ ID NO: 5 | 1512 bp |

| | |
|---|---|
| NOV1c, | ATGAACACTTTTACCTGGACCCTAGGTGTGCTATTCTTCCTACTAGTGGACACTGGAC |
| CG57067-02 | |
| DNA Sequence | ATTGCAGAGGTGGACAATTCAAAATTAAAAAAATAAACCAGAGAAGATACCCTCGTGC |
| | CACAGATGGTAAAGAGGAAGCAAAGAAATGTGCATACACATTCCTGGTACCTGAACAA |
| | AGAATAACAGGGCCAATCTGTGTCAACACCAAGGGGCAAGATGCAAGTACCATTAAAG |
| | ACATCATCACCAGGATGGACCTTGAAAACCTGAAGGATGTGCTCTCCAGGCAGAAGCG |
| | GGAGATAGATGTTCTGCAACTGGTGGTGGATGTAGATGGAAACATTGTGAATGAGGTA |
| | AAGCTGCTGAGAAAGGAAAGCCGTAACATGAACTCTCGTGTTACTCAACTCTATATGC |
| | AATTATTACATGAGATTATCCGTAAGAGGGATAATTCACTTGAACTTTCCCAACTGGA |
| | AAACAAAATCCTCAATGTCACCACAGAAATGTTGAAGATGGCAACAAGATACAGGGAA |
| | CTAGAGGTGAAATACGCTTCCTTGACTGATCTTGTCAATAACCAATCTGTGATGATCA |
| | CTTTGTTGGAAGAACAGTGCTTGAGGATATTTTCCCGACAAGACACCCATGTGTCTCC |
| | CCCACTTGTCCAGGTGGTGCCACAACATATTCCTAACAGCCAACAGTATACTCCTGGT |
| | CTGCTGGGAGGTAACGAGATTCAGAOGGATCCAGGTTATCCCAGAGATTTAATGCCAC |
| | CACCTGATCTGGCAACTTCTCCCACCAAAAGCCCTTTCAAGATACCACCGGTAACTTT |
| | CATCAATGAAGGACCATTCAAAGACTGTCAGCAAGCAAAAGAAGCTGGGCATTCGGTC |
| | AGTGGGATTTATATGATTAAACCTGAAAACAGCAATGGACCAATGCAGTTATGGTGTG |
| | AAAACAGTTTGGACCCTGGGGGTTGGACTGTTATTCAGAAAAGAACAGACGGCTCTGT |
| | CAACTTCTTCAGAAATTGGGAAAATTATAAGAAAGGGTTTGGAAACATTGACGGAGAA |
| | TACTGGCTTGGACTGGAAAATATCTATATGCTTAGCAATCAAGATAATTACAAGTTAT |
| | TGATTGAATTAGAAGACTGGAGTGATAAAAAAGTCTATGCAGAATACAGCAGCTTTCG |
| | TCTGGAACCTGAAAGTGAATTCTATAGACTGCGCCTGGGAACTTACCAGGGAAATGCA |
| | GGGGATTCTATGATGTGGCATAATGGTAAACAATTCACCACACTGGACAGAGATAAAG |
| | ATATGTATGCAGGAAACTGCGCCCACTTTCATAAAGGAGGCTGGTGGTACAATGCCTG |
| | TGCACATTCTAACCTAAATGGAGTATGGTACAGAGGAGGCCATTACAGAAGCAAGCAC |
| | CAAGATGGAATTTTCTGGGCCGAATACAGAGGCGGGTCATACTCCTTAAGAGCAGTTC |
| | AGATGATGATCAAGCCTATTGACAAGGGCAATTCTGCAGATATCCAGCACAGTGGCGG |
| | CCGC |

| | |
|---|---|
| ORF Start: ATG at 1 | ORF Stop: end of sequence |

| | | |
|---|---|---|
| SEQ ID NO: 6 | 504 aa | MW at 58027.5 kD |

| | |
|---|---|
| NOV1c, | MKTFTWTLGVLFFLLVDTGHCRGGQFKIKKINQRRYPRATDGKEEAKKCAYTFLVPEQ |
| CG57067-02 | |
| Protein | RITGPICVNTKGQDASTIKDMITRMDLENLKDVLSRQKREIDVLQLVVDVDGNIVNEV |
| Sequence | |
| | KLLRKESRNMNSRVTQLYMQLLHEIIRKRDNSLELSQLENKILNVTTEMLKMATRYRE |
| | LEVKYASLTDLVNNQSVMITLLEEQCLRIFSRQDTHVSPPLVQVVPQHIPNSQQYTPG |
| | LLGGNEIQRDPGYPRDLMPPPDLATSPTKSPFKIPPVTFINEGPFKDCQQAKEAGHSV |
| | SGIYMIKPENSNGPMQLWCENSLDPGGWTVIQKRTDGSVNFFRNWENYKKGFGNIDGE |
| | YWLGLENIYMLSNQDNYKLLIELEDWSDKKVYAEYSSFRLEPESEFYRLRLGTYQGNA |
| | GDSMMWHNGKQFTTLDRDKDMYAGNCAHFHKGGWWYNACAHSNLNGVWYRGQHYRSKH |
| | QDGIFWAEYRGGSYSLRAVQMMIKPIDKGNSADIQHSGGR |

| | |
|---|---|
| SEQ ID NO:7 | 1377bp |

| | |
|---|---|
| NOV1d, | AAGATACCACCGGTAACTTTCATCAATGAAGGACCATTCAAAGACTGTCAGCAAGCAA |
| CG57067-03 | |
| DNA Sequence | AAGAAGCTGGGCATTCGGTCAGTGGGATTTATATGATTAAACCTGAAAACAGCAATGG |
| | ACCAATGCAGTTATGGTGTGAAAACAGTTTGGACCCTCGGGGTTGGACTGTTATTCAG |
| | AAAAGAACAGACGGCTCTGTCAACTTCTTCAGAAATTGGGAAAATTATAAGAAAGGGT |
| | TTGGAAACATTGACGGAGAATACTGGCTTGGACTGGAAAATATCTATATGCTTAGCAA |
| | TCAAGATAATTACAAGTTATTGATTGAATTAGAAGACTGGAGTGATAAAAAAGTCTAT |
| | GCAGAATACAGCAGCTTTCGTCTGGAACCTGAAAGTGAATTCTATAGACTGCGCCTGG |
| | GAACTTACCAGGGAAATGCAGCGGATTCTATGATGTGGCATAATGGTAAACAATTCAC |
| | CACACTGGACAGAGATAAAGATATGTATGCAGGAAACTGCGCCCACTTTCATAAAGGA |
| | GGCTGGTGGTACAATGCCTGTGCACATTCTAACCTAAATGGAGTATGGTACAGAGGAG |
| | GCCATTACAGAAGCAAGCACCAAGATGGAATTTTCTGGGCCGAATACAGAGGCGGGTC |
| | ATACTCCTTAAGAGCAGTTCAGATGATGATCAAGCCTATTGACGAGCCCAAATCTTGT |
| | GACAAAACTCACACATGCCCACCGTGCCCAGCACCTGAACTCCTGGGGGGACCGTCAG |
| | TCTTCCTCTTCCCCCCAAAACCCAAGGACACCCTCATGATCTCCCGGACCCCTGAGGT |
| | CACATGCGTGGTGGTGGACGTGAGCCACGAAGACCCTGAGGTCAAGTTCAACTGGTAC |
| | GTGGACGGCGTGGAGGTGCATAATGCCAAGACAAAGCCGCGGGAGGAGCAGTACAACA |
| | GCACGTACCGTGTGGTCAGCGTCCTCACCGTCCTGCACCAGGACTGGCTGAATGGCAA |
| | GGAGTACAAGTGCAAGGTCTCCAACAAAGCCCTCCCAGCCCCCATCGAGAAAACCATC |
| | TCCAAAGCCAAAGGGCAGCCCCGAGAACCACAGGTGTACACCCTGCCCCCATCCCGGG |
| | AGGAGATGACCAAGAACCAGGTCAGCCTGACCTGCCTGGTCAAAGGCTTCTATCCCAG |
| | CGACATCGCCGTGGAGTGGGAGAGCAATGGGCAGCCGGAGAACAACTACAAGACCACG |
| | CCTCCCGTGCTGGACTCCGACGGCTCCTTCTTCCTCTATAGCAAGCTCACCGTGGACA |
| | AGAGCAGGTGGCAGCAGGGGAACGTCTTCTCATGCTCCGTGATGCATGAGGCTCTGCA |
| | CAACCACTACACGCAGAAGAGCCTCTCCCTGTCCCCGGGTAAA |

| | |
|---|---|
| ORF Start: at 1 | ORF Stop: end of sequence |

| | | |
|---|---|---|
| SEQ ID NO:8 | 469aa | MW at 52501.8 kD |

| | |
|---|---|
| NOV1d, | KIPPVTFINEGPFKDCQQAKEAGHSVSGIYMIKPENSNGPMQLWCENSLDPGGWTVIQ |
| CG57067-03 | |
| Protein | KRTDGSVNFFRNWENYKKGFGNIDGEYWLGLENIYMLSNQDNYKLLIELEDWSDKKVY |
| Sequence | |
| | AEYSSFRLEPESEFYRLRLGTYQGNAGDSMMWHNGKQFTTLDRDKDMYAGNCAHFHKG |
| | GWWYNACAHSNLNGVWYRGGHYRSKHQDGIFWAEYRGGSYSLRAVQMMIKPIDEPKSC |
| | DKTHTCPPCPAPELLGGPSVFLFPPKPKDTLMISRTPEVTCVVVDVSHEDPEVKENWY |
| | VDGVEVHNAKTKPREEQYNSTYRVVSVLTVLHQDWLNGKEYKCKVSNKALPAPIEKTI |
| | SKAKGQPREPQVYTLPPSREEMTKNQVSLTCLVKGFYPSDIAVEWESNGQPENNYKTT |
| | PPVLDSDGSFFLYSKLTVDKSRWQQGNVFSCSVMHEALHNHYTQKSLSLSPGK |

| | |
|---|---|
| SEQ ID NO:9 | 1377bp+TZ,1/46 |

| | |
|---|---|
| NOV1e, | AAGATACCACCGGTAACTTTCATCAATGAAGQACCATTCAAAGACTGTCAGCAAGCAA |
| CG57067-04 | |
| DNA Sequence | AAGAAGCTGGGCATTCGGTCAGTGGGATTTATATGATTAAACCTGAAAACAGCAATGG |
| | ACCAATGCAGTTATGGTGTGAAAACAGTTTGGACCCTGGGGGTTGGACTGTTATTCAG |
| | AAAAGAACAGACGGCTCTGTCAACTTCTTCAGAAATTGGGAAAATTATAAGAAAGGGT |
| | TTGGAAACATTGACGGAGAATACTGGCTTGGACTGGAAAATATCTATATGCTTAGCAA |
| | TCAAGATAATTACAAGTTATTGATTGAATTAGAAGACTGGAGTGATAAAAAAGTCTAT |
| | GCAGAATACAGCAGCTTTCGTCTGGAACCTGAAAGTGAATTCTATAGACTGCGCCTGG |
| | GAACTTACCAGQGAAATGCAGGGGATTCTATGATGTGGCATAATGGTAAACAATTCAC |
| | CACACTGGACAGAGATAAAGATATGTATGCAGGAAACTGCGCCCACTTTCATAAAGGA |
| | GGCTGGTGGTACAATGCCTGTGCACATTCTAACCTAAATGGAGTATGGTACAGAGGAG |
| | GCCATTACAGAAGCAAGCACCAAGATGGAATTTTCTGGGCCGAATACAGAGGCGGGTC |
| | ATACTCCTTAAGAGCAGTTCAGATOATGATCAAGCCTATTGACGAGCCCAAATCTTGT |
| | GACAAAACTCACACATGCCCACCGTGCCCAGCACCTGAACTCCTGGGGGGACCGTCAG |
| | TCTTCCTCTTCCCCCCAAAACCCAAGGACACCCTCATGATCTCCCGGACCCCTGAGGT |
| | CACATGCGTGGTGGTGGACGTGAGCCACGAAGACCCTGAGGTCAAGTTCAACTGGTAC |
| | GTGGACGGCGTGGAGGTGCATAATGCCAAGACAAAGCCGCGGGAGGAGCAGTACAACA |
| | GCACGTACCGTGTGGTCAGCGTCCTCACCGTCCTGCACCAGGACTGGCTGAATGGCAA |
| | GGAGTACAAGTGCAAGGTCTCCAACAAAGCCCTCCCAGCCCCCATCGAGAAAACCATC |
| | TCCAAAGCCAAAGGGCAGCCCCGAGAACCACAGGTGTACACCCTGCCCCCATCCCGGG |
| | AGGAGATGACCAAGAACCAGGTCAGCCTGACCTGCCTGGTCAAAGGCTTCTATCCCAG |
| | CGACATCGCCGTGGAGTGGGAGAGCAATGGGCAGCCGGAGAACAACTACAAGACCACG |
| | CCTCCCGTGCTGGACTCCGACGGCTCCTTCTTCCTCTATAGCAAGCTCACCGTGGACA |
| | AGAGCAGGTGGCACCAGGGGAACGTCTTCTCATGCTCCGTGATGCATGAGGCTCTGCA |
| | CAACCACTACACGCAGAAGAGCCTCTCCCTGTCCCCGGGTAAA |

| | |
|---|---|
| ORF Start: at 1 | ORF Stop: at 1375 |

| | | |
|---|---|---|
| SEQ ID NO: 10 | 468 aa | MW at 523 73.6 kD |

| | |
|---|---|
| NOV1e, | KIPPVTFINEGPFKDCQQAKEAGHSVSGIYMIKPENSNGPMQLWCENSLDPGGWTVIQ |
| CG57067-04 | |
| Protein | KRTDGSVNFFRNWENYKKGFGNIDGEYWLGLENIYMLSNQDNYKLLIELEDWSDKKVY |
| Sequence | |
| | AEYSSFRLEPESEFYRLRLGTYQGNAGDSMMWHNGKQFTTLDRDKDMYAGNCAHFHKG |
| | GWWYNACAHSNLNGVWYRGGHYRSKHQDGIFWAEYRGGSYSLRAVQMMIKPIDEPKSC |
| | DKTHTCPPCPAPELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKVSNKALPAPIEKTI |
| | SKAKGQPREPQVYTLPPSREEMTKNQVSLTCLVKGFYPSDIAVEWESNGQPENNYKTT |
| | PPVLDSDGSFFLYSKLTVDKSRWQQGNVFSCSVMHEALHNHYTQKSLSLSPG |

| | |
|---|---|
| SEQ ID NO: 11 | 1377bp |

| | |
|---|---|
| NOV1F, | GAGCCCAAATCTTGTGACAAAACTCACACATGCCCACCGTGCCCAGCACCTGAACTCC |
| CG57067-05 | |
| DNA Sequence | TGGGGGGACCGTCAGTCTTCCTCTTCCCCCCAAAACCCAAGGACACCCTCATGATCTC |
| | CCGGACCCCTGAGGTCACATGCGTGGTGGTGGACGTGAGCCACGAAGACCCTGAGGTC |
| | AAGTTCAACTGGTACGTGGACGGCGTGGAGGTGCATAATGCCAAGACAAAGCCGCGGC |
| | AGGAGCAGTACAACAGCACGTACCGTGTGGTCAGCGTCCTCACCGTCCTGCACCAGGA |
| | CTGGCTGAATGGCAAGGAGTACAAGTGCAAGGTCTCCAACAAAGCCCTCCCAGCCCCC |
| | ATCGAGAAAACCATCTCCAAAGCCAAAGGGCAGCCCCGAGAACCACAGGTGTACACCC |
| | TGCCCCCATCCCGGGAGGAGATGACCAAGAACCAc4GTCAGCCTGACCTGCCTGGTCAA |
| | AGGCTTCTATCCCAGCGACATCGCCGTGGAGTGGGAGAGCAATGGGCAGCCGGAGAAC |
| | AACTACAAGACCACGCCTCCCGTGCTGGACTCCGACGGCTCCTTCTTCCTCTATAGCA |
| | AGCTCACCGTGGACAAGAGCAGGTGGCACCAGCCGAACGTCTTCTCATGCTCCGTGAT |
| | GCATGAGGCTCTGCACAACCACTACACGCAGAAGAGCCTCTCCCTGTCCCCGGGTAAA |
| | AAGATACCACCGGTAACTTTCATCAATGAAGGACCATTCAAAGACTGTCAGCAAGCAA |
| | AAGAAGCTGGGCATTCGGTCAGTGGGATTTATATGATTAAACCTGAAAACAGCAATGG |
| | ACCAATGCAGTTATGGTGTGAAAACAGTTTGGACCCTGGGGGTTGGACTGTTATTCAG |
| | AAAAGAACAGACGGCTCTGTCAACTTCTTCAGAAATTGGGAAAATTATAAGAAAGGGT |
| | TTGGAAACATTGACGGAGAATACTGGCTTGGACTGGAAAATATCTATATGCTTAGCAA |
| | TCAAGATAATTACAAGTTATTGATTGAATTAGAAGACTGGAGTGATAAAAAAGTCTAT |
| | GCAGAATACAGCAGCTTTCGTCTGGAACCTGAAAGTGAATTCTATAGACTGCGCCTGG |
| | GAACTTACCAGGGAAATGCAGGGGATTCTATGATGTGGCATAATGGTAAACAATTCAC |
| | CACACTGGACAGAGATAAAGATATGTATGCAGGAAACTGCGCCCACTTTCATAAAGGA |
| | GGCTGGTGGTACAATGCCTGTGCACATTCTAACCTAAATGGAGTATGGTACAGAGGAG |
| | GCCATTACAGAAGCAAGCACCAAGATGGAATTTTCTGGGCCGAATACAGAGGCGGGTC |
| | ATACTCCTTAAGAGCAGTTCAGATGATGATCAAGCCTATTGAC |

| | |
|---|---|
| ORF Start: at 1 | ORF Stop: end of sequence |

| | | |
|---|---|---|
| SEQ ID NO: 12 | 469aa | MW at 52501.8 kD |

| | |
|---|---|
| NOV1f, | EPKSCDKTHTCPPCPAPELLGGPSVFLFPPKPKDTLMISRTPEVTCVVVDVSHEDPEV |
| CG57067-05 | |
| Protein | KFNWYVDGVEVHNAKTKPREEQYNSTYRVVSVLTVLHQDWLNGKEYKCKVSNKALPAP |
| Sequence | |
| | IEKTISKAKGQPREPQVYTLPPSREEMTKMQVSLTCLVKGFYPSDIAVEwESNGQPEN |
| | NYKTTPPVLDSDGSFFLYSKLTVDKSRWQQGNVFSCSVMHEALHNHYTQKSLSLSPGK |
| | KIPPVTFINEGPFKDCQQAKEAGHSVSGIYMIKPENSNGPMQLWCENSLDPGGWTVIQ |
| | KRTDGSVNFFRNWENYKKGFGNIDGEYWLGLENIYMLSNQDNYKLLIELEDWSDKKVY |
| | AEYSSFRLEPESEFYRLRLGTYQGNAGDSMMWHNGKQFTTLDRDKDMYAGNCAHFHKG |
| | GWWYNACAHSNLNGVWYRGGHYRSKHQDGIFWAEYRGGSYSLRAVQMMIKPID |

| | |
|---|---|
| SEQ ID NO: 13 | 1473 bp |

| | |
|---|---|
| NOV1g, | ATGAAGACTTTTACCTGGACCCTAGGTGTGCTATTCTTCCTACTAGTGGACACTGGAC |
| CG57067-06 | |
| DNA Sequence | ATTGCAGAGGTGGACAATTCAAAATTAAAAAAATAAACCAGAGAAGATACCCTCGTGC |
| | CACAGATGGTAAAGAGGAAGCAAAGAAATGTGCATACACATTCCTGGTACCTGAACAA |
| | AGAATAACAGGGCCAATCTGTGTCAACACCAAGGGGCAAGATGCAAGTACCATTAAAG |
| | ACATGATCACCAGGATGGACCTTGAAAACCTGAAGGATGTGCTCTCCAGCCAGAAGCG |
| | GGAGATAGATGTTCTGCAACTGGTGGTGGATGTAGATGGAAACATTGTGAATGAGGTA |
| | AGCTGCTGAGAAAGGAAAGCCGTAACATGAACTCTCGTGTTACTCAACTCTATATGC |
| | AATTATTACATGAGATTATCCGTAAGAGGGATAATTCACTTGAACTTTCCCAACTGGA |
| | AAACAAAATCCTCAATGTCACCACAGAAATGTTGAAGATGGCAACAAGATACAGGGAA |
| | CTAGAGGTGAAATACGCTTCCTTGACTGATCTTGTCAATAACCAATCTGTGATGATCA |
| | CTTTGTTGGAAGAACAGTGCTTGAGGATATTTTCCCGACAAGACACCCATGTGTCTCC |
| | CCCACTTGTCCAGGTGGTGCCACAACATATTCCTAACAGCCAACAGTATACTCCTGGT |
| | CTGCTCCGAGGTAACGAGATTCAGAGGGATCCAGGTTATCCCAGAGATTTAATGCCAC |
| | CACCTGATCTGGCAACTTCTCCCACCAAAAGCCCTTTCAAGATACCACCGGTAACTTT |
| | CATCAATGAAGGACCATTCAAAGACTGTCAGCAAGCAAAAGAAGCTGGGCATTCGGTC |
| | AGTGGQATTTATATGATTAAACCTGAAAACAGCAATGGACCAATGCAGTTATGGTGTG |
| | AAAACAGTTTGGACCCTGGGGGTTGGACTGTTATTCAGAAAAGAACAGACGGCTCTGT |
| | CAACTTCTTCAGAAATTGGGAAAATTATAAGAAAGGGTTTGGAAACATTGACGGAGAA |
| | TACTGGCTTGGACTGGAAAATATCTATATGCTTAGCAATCAAGATAATTACAAGTTAT |
| | TGATTGAATTAGAAGACTGGAGTGATAAAAAAGTCTATGCAGAATACAGCAGCTTTCG |
| | TCTGGAACCTGAAAGTGAATTCTATAGACTGCGCCTGGGAACTTACCAGGGAAATGCA |
| | GGGGATTCTATGATGTGGCATAATGGTAAACAATTCACCACACTGGACAGAGATAAAG |
| | ATATGTATGCAGGAAACTGCGCCCACTTTCATAAAGGAGGCTGGTGGTACAATGCCTG |
| | TGCACATTCTAACCTAAATGGAGTATGGTACAGAGGAGGCCATTACAGAAGCAAGCAC |
| | CAAGATGGAATTTTCTGGGCCGAATACAGAGGCGGGTCATACTCCTTAAGAGCAGTTC |
| | AGATGATGATCAAGCCTATTGAC |

| | |
|---|---|
| ORF Start: ATG at 1 | ORF Stop: end of sequence |

| | | |
|---|---|---|
| SEQ ID NO: 14 | 491 aa | MW at 567 19.2 kD |

| | |
|---|---|
| NOV1g, | MKTFTWTLGVLFFLLVDTGHCRGGQFKIKKINQRRYPRATDGKEEAKKCAYTFLVPEQ |
| CG57067-06 | |
| Protein | RITGPICVNTKGQDASTIKDMITRMDLENLKDVLSRQKREIDVLQLVVDVDGNIVNEV |
| Sequence | |
| | KLLRKESRNMNSRVTQLYMQLLHEIIRKRDNSLELSQLENKILNVTTEMLKMATRYRE |
| | LEVKYASLTDLVNNQSVMITLLEEQCLRIFSRQDTHVSPPLVQVVPQHIPNSQQYTPG |
| | LLGGNEIQRDPGYPRDLMPPPDLATSPTKSPFKIPPVTFINEGPFKDCQQAKEAGHSV |
| | SGIYMIKPENSNGPMQLWCENSLDPGGWTVIQKRTDGSVNFFRNWENYKKGFGNIDGE |
| | YWLGLENIYMLSNQDNYKLLIELEDWSDKKVYAEYSSFRLEPESEFYRLRLGTYQGNA |
| | GDSMMWHNGKQFTTLDRDKDMYAONCAHFHKGGWWYNACAHSNLNGVWYRGGHYRSKH |
| | QDGIFWAEYRGGSYSLRAVQMMIKPID |

| | |
|---|---|
| SEQ ID NO: 15 | 1492bp |

| | |
|---|---|
| NOV1h, | TGCAGAATTCGCCCTTATGAAGACTTTTACCTGGACCCTAGGTGTGCTATTCTTCCTA |
| CG57067-07 | |
| DNA Sequence | CTAGTGGACACTGGACATTGCAGAGGTGGACAATTCAAAATTAAAAAAATAAACCAGA |
| | GAAGATACCCTCGTGCCACAGATGGTAAAGAGGAAGCAAAGAAATGTGCATACACATT |
| | CCTGGTACCTGAACAAAGAATAACAGGGCCAATCTGTGTCAACACCAAGGGGCAAGAT |
| | GCAAGTACCATTAAAGACATGATCACCAGGATGGACCTTGAAAACCTGAAGGATGTGC |
| | TCTCCAGGCAGAAGCGGGAGATAGATGTTCTGCAACTGGTGGTGGATGTAGATGGAAA |
| | CATTGTGAATGAGGTAAAGCTGCTGAGAAAGGAAAGCCGTAACATGAACTCTCGTGTT |
| | ACTCAACTCTATATGCAATTATTACATGAGATTATCCGTAAGAGGGATAATTCACTTG |
| | AACTTTCCCAACTGGAAAACAAAATCCTCAATGTCACCACAGAAATGTTGAAGATGGC |
| | AACAAGATACAGGGAACTAGAGGTGAAATACGCTTCCTTGACTGATCTTGTCAATAAC |
| | CAATCTGTGATGATCACTTTGTTGGAAGAACAGTGCTTGAGGATATTTTCCCGACAAG |
| | GCACCCATGTGTCTCCCCCACTTGTCCAGGTGGTGCCACAACATATTCCTAACAGCCA |
| | ACAGTATACTCCTGGTCTGCTGGGAGGTAACGAGATTCAGAGGGATCCAGGTTATCCC |
| | AGAGATTTAATGCCACCACCTGATCTGGCAACTTCTCCCACCAAAAGCCCTTTCAAGA |
| | TACCACCGGTAACTTTCATCAATGAAGGACCATTCAAAGACTGTCAGCAAGCAAAAGA |
| | AGCTGGGCATTCGGTCAGTGGGATTTATATGATTAAACCTGAAAACAGCAATGGACCA |
| | ATGCAGTTATGGTGTGAAAACAGTTTGGACCCTGGGGGTTGGACTGTTATTCAGAAAA |
| | GAACAGACGGCTCTGTCAACTTCTTCAGAAATTGGGAAAATTATAAGAAAGGGTTTGG |
| | AAACATTGACGGAGAATACTGGCTTGGACTGGAAAATATCTATATGCTTAGCAATCAA |
| | GATAATTACAAGTTATTGATTGAATTAGAAGACTGGAGTGATAAAAAAGTCTATGCAG |
| | AATACAGCAGCTTTCGTCTGGAACCTGAAAGTGAATTCTATAGACTGCGCCTGGGAAC |
| | TTACCAGGGAAATGCAGGGGATTCTATGATGTGGCATAATGGTAAACAATTCACCACA |
| | CTQGACAGAGATAAAGATATGTATGCAGGAAACTGCGCCCACTTTCATAAAGGAGGCT |
| | GGTGGTACAATGCCTGTGCACATTCTAACCTAAATGGAGTATGGTACAGAGGAGGCCA |
| | TTACAGAAGCAAGCACCAAGATGGAATTTTCTGGGCCGAATACAGAGGCGGGTCATAC |
| | TCCTTAAGAGCAGTTCAGATGATGATCAAGCCTATTGACTGA |

| | |
|---|---|
| ORF Start: ATG at 17 | ORF Stop: TGA at 1490 |

| | | |
|---|---|---|
| SEQ ID NO: 16 | 491 aa | MW at 56661.1 kD |

| | |
|---|---|
| NOV1h, | MKTFTWTLGVLFFLLVDTGHCRGGQFKIKKINQRRYPRATDGKEEAKKCAYTFLVPEQ |
| CG57067-07 | |
| Protein | RITGPICVNTKGQDASTIKDMITRMDLENLKDVLSRQKREIDVLQLVVDVDGNIVNEV |
| Sequence | |
| | KLLRKESRNMNSRVTQLYMQLLHEIIRKRDNSLELSQLENKILNVTTEMLKMATRYRE |
| | LEVKYASLTDLVNNQSVMITLLEEQCLRIFSRQGTHVSPPLVQVVPQHIPNSQQYTPG |
| | LLGGNEIQRDPGYPRDLMPPPDLATSPTKSPFKIPPVTFINEGPFKDCQQAKEAGHSV |
| | SGIYMIKPENSNGPMQLWCENSLDPGGWTVIQKRTDGSVNFFRNWENYKKGFGNIDGE |
| | YWLGLENIYMLSNQDNYKLLIELEDWSDKKVYAEYSSFRLEPESEFYRLRLGTYQGNA |
| | GDSMMWHNGKQFTTLDRDKDMYAGNCAHFHKGGWWYNACAHSNLNGVWYRGGHYRSKH |
| | QDGIFWAEYRGGSYSLRAVQMMIKPID |

| | |
|---|---|
| SEQ ID NO: 17 | 1380 bp |

| | |
|---|---|
| NOV1i, | AAGATACCACCGGTAACTTTCATCAATGAAGGACCATTCAAAGACTGTCAGCAAGCAA |
| CG57067-08 | |
| DNA Sequence | AAGAAGCTGGGCATTCGGTCAGTGGGATTTATATGATTAAACCTGAAAACAGCAATGG |
| | ACCAATGCAGTTATGGTGTGAAAACAGTTTGGACCCTGGGGGTTGGACTGTTATTCAG |
| | AAAAGAACAGACGGCTCTGTCAACTTCTTCAGAAATTGGGAAAATTATAAGAAAGGGT |
| | TTGGAAACATTGACGGAGAATACTGCCTTGGACTGGAAAATATCTATATGCTTAGCAG |
| | TCAAGATAATTACAAGTTATTGATTGAATTAGAAGACTGGAGTGATAAAAAAGTCTAT |
| | GCAGAATACAGCAGCTTTCGTCTGGAACCTGAAAGTGAATTCTATAGACTGCGCCTGG |
| | GAACTTACCAGGGAAATGCAGGGGATTCTATGATGTGGCATAATGGTAAACAATTCAC |
| | CACACTGGACAGAGATAAAGATATGTATGCAGGAAACTGCGCCCACTTTCATAAAGGA |
| | GGCTGGTGGTACAATGCCTGTGCACATTCTAACCTAAATGGAGTATGGTACAGAGGAG |
| | GCCATTACAGAAGCAAGCACCAAGATGGAATTTTCTGGGCCGAATACAGAGGCGGGTC |
| | ATACTCCTTAAGAGCAGTTCAGATGATGATCAAGCCTATTGACGAGCCCAAATCTTGT |
| | GACAAAACTCACACATGCCCACCGTGCCCAGCACCTGAACTCCTGGGGCGACCGTCAG |
| | TCTTCCTCTTCCCCCCAAAACCCAAGGACACCCTCATGATCTCCCGGACCCCTGAGGT |
| | CACATGCGTGGTGGTGGACGTGAGCCACGAAGACCCTGAGGTCAAGTTcAACTGGTAC |
| | GTGGACGGCGTGGAGGTGCATAATGCCAAGACAAAGCCGCGGGAGGAGCAGTACAACA |
| | GCACGTACCGTGTGGTCAGCGTCCTCACCGTCCTGCACCAGGACTGGCTGAATGGCAA |
| | GGAGTACAAGTGCAAGGTCTCCAACAAAGCCCTCCCAGCCCCCATCGAGAAAACCATC |
| | TCCAAAGCCAAAGGGCAGCCCCGAGAACCACAGGTGTACACCCTGCCCCCATCCCGGG |
| | AQGAGATGACCAAGAACCAGGTCAGCCTGACCTGCCTGGTCAAAGGCTTCTATCCCAG |
| | CGACATCGCCGTGGAGTGGGAGAGCAATGGGCACCCGGAGAACAACTACAAGACCACG |
| | CCTCCCGTGCTGGACTCCGACGGCTCCTTCTTCCTCTATAGCAAGCTCACCGTGGACA |
| | ACAGCAGGTGGCAGCAGGGGAACGTCTTCTCATGCTCCGTGATGCATGAGGCTCTGCA |
| | CAACCACTACACGCAGAAGAGCCTCTCCCTGTCCCCGGGTAAATGA |

| | |
|---|---|
| ORF Start: at 1 | ORF Stop: TGA at 1378 |

| | | |
|---|---|---|
| SEQ ID NO: 18 | 469aa | MW at 52501.8 kD |

| | |
|---|---|
| NOV1i, | KIPPVTFINEGPFKDCQQAKEAGHSVSGIYMIKPENSNGPMQLWCENSLDPGGWTVIQ |
| CG57067-08 | |
| Protein | KRTDGSVNFFRNWENYKKGFGNIDGEYWLGLENIYMLSNQDNYKLLIELEDWSDKKVY |
| Sequence | |
| | AEYSSFRLEPESEFYRLRLGTYQGNAGDSMMWHNGKQFTTLDRDKDMYAGNCAHFHKG |
| | GWWYNACAHSNLNGVWYRGGHYRSKHQDGIFWAEYRGGSYSLRAVQMMIKPIDEPKSC |
| | DKTHTCPPCPAPELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKVSNKALPAPIEKTI |
| | SKAKGQPREPQVYTLPPSREEMTKNQVSLTCLVKGFYPSDIAVEWESNGQPENNYKTT |
| | PPVLDSDGSFFLYSKLTVDKSRWQQGNVFSCSVMHEAILHNHYTQKSLSLSPGK |

| | |
|---|---|
| SEQ ID NO: 19 | 2181 bp |

| | |
|---|---|
| NOV1j, | ATGAAGACTTTTACCTGGACCCTAGGTGTGCTATTCTTCCTACTAGTGGACACTGGAC |
| CG57067-09 | |
| DNA Sequence | ATTGCAGAGGTGGACAATTCAAAATTAAAAAAATAAACCAGAGAAGATACCCTCGTGC |
| | CACAGATGGTAAAGAGGAAGCAAAGAAATGTGCATACACATTCCTGGTACCTGAACAA |
| | AGAATAACAGGGCCAATCTGTGTCAACACCAAGGGGCAAGATGCAAGTACCATTAAAG |
| | ACATGATCACCAGGATGGACCTTGAAAACCTGAAGGATGTGCTCTCCAGGCAGAAGCG |
| | GGAGATAGATGTTCTGCAACTGGTGGTGGATGTAGATGGAAACATTGTGAATGAGGTA |
| | AAGCTGCTGAGAAAGGAAAGCCGTAACATGAACTCTCGTGTTACTCAACTCTATATGC |
| | ATTATTACATGAGATTATCCGTAAGAGGGATAATTCACTTGAACTTTCCCAACTGGAA |
| | AAACAAAATCCTCAATGTCACCACAGAAATGTTGAAGATGGCAACAAGATACAGGGAA |
| | CTAGAGGTGAAATACGCTTCCTTGACTGATCTTGTCAATAACCAATCTGTGATGATCA |
| | CTTTGTTGGAAGAACAGTGCTTGAGGATATTTTCCCGACAAGACACCCATGTGTCTCC |
| | CCCACTTGTCCAGGTGGTGCCACAACATATTCCTAACAGCCAACAGTATACTCCTGGT |
| | CTGCTGGGAGGTAACGAGATTCAGAGGGATCCAGGTTATCCCAGAGATTTAATGCCAC |
| | CACCTGATCTGGCAACTTCTCCCACCAAAAGCCCTTTCAAGATACCACCCGTAACTTT |
| | CATCAATGAAGGACCATTCAAAGACTGTCAGCAAGCAAAAGAAGCTGGGCATTCGGTC |
| | AGTGGGATTTATATGATTAAACCTGAAAACAGCAATGGACCAATGCAGTTATGGTGTG |
| | AAAACAGTTTGGACCCTGGGGGTTGGACTGTTATTCAGAAAAGAACAGACGGCTCTGT |
| | CAACTTCTTCAGAAATTGGGAAAATTATAAGAAAGGGTTTGGAAACATTGACGGAGAA |
| | TACTGGCTTGGACTGGAAAATATCTATATGCTTAGCAATCAAGATAATTACAAGTTAT |
| | TGATTGAATTAGAAGACTGGAGTGATAAAAAAGTCTATGCAGAATACAGCAGCTTTCG |
| | TCTGGAACCTGAAAQTGAATTCTATAGACTGCGCCTGGGAACTTACCAGGGAAATGCA |
| | GGGGATTCTATGATGTGGCATAATGGTAAACAATTCACCACACTGGACAGAGATAAAG |
| | ATATGTATGCAGGAAACTGCGCCCACTTTCATAAAGGAGGCTGGTGGTACAATGCCTG |
| | TGCACATTCTAACCTAAATGGAGTATGGTACAGAGGAGGCCATTACAGAAGCAAGCAC |
| | CAAGATGGAATTTTCTGGGCCGAATACAGAGGCGGGTCATACTCCTTAAGAGCAGTTC |
| | AGATGATGATCAAGCCTATTGACGGCGGCGGCGGCGGCGGCGGCGGCGACAAAACTCA |
| | CACATGCCCACCGTGCCCAGCACCTGAACTCCTGGGQGGACCGTCAGTCTTCCTCTTC |
| | CCCCCAAAACCCAAGGACACCCTCATGATCTCCCGGACCCCTGAGGTCACATGCGTGG |
| | TGGTGGACGTGAGCCACGAAGACCCTGAGGTCAAGTTCAACTGGTACGTGGACGGCGT |
| | GGAGGTGCATAATGCCAAGACAAAGCCGCGGGAGGAGCAGTACAACAGCACGTACCGT |
| | GTGGTCAGCGTCCTCACCGTCCTGCACCAGGACTGGCTGAATGGCAAGGAGTACAAGT |
| | GCAAGGTCTCCAACAAAGCCCTCCCAGCCCCCATCGAGAAAACCATCTCCAAAGCCAA |
| | AGGGCAGCCCCGAGAACCACAGGTGTACACCCTGCCCCCATCCCGGGATGAGCTGACC |
| | AAGAACCAGGTCAGCCTGACCTGCCTGGTCAAAGGCTTCTATCCCAGCGACATCGCCG |
| | TGGAGTGGGAGAGCAATGGGCAGCCGGAGAACAACTACAAGACCACGCCTCCCGTGCT |
| | GGACTCCGACGGCTCCTTCTTCCTCTACAGCAAGCTCACCGTGGACAAGAGCAGGTGG |
| | CAGCAGGGGAACGTCTTCTCATGCTCCGTGATGCATGAGGCTCTGCACAACCACTACA |
| | CGCAGAAGAGCCTCTCCCTGTCTCCGGGTAAATGA |

| | |
|---|---|
| ORF Start: ATG at 1 | ORF Stop: TGA at 2179 |

| | | |
|---|---|---|
| SEQ ID NO: 20 | 726 aa | MW at 82705.4 kD |

| | |
|---|---|
| NOV1j, | MKTFTWTLGVLFFLLVDTGHCRGGQFKIKKINQRRYPRATDCKEEAKKCAYTFLVPEQ |
| CG57067-09 | |
| Protein | RITGPICVNTKGQDASTIKDMITRMDLENLKDVLSRQKREIDVLQLVVDVDGNIVNEV |
| Sequence | |
| | KLLRKESRNMNSRVTQLYMQLLHEIIRKRDNSLELSQLENKILNVTTEMLKMATRYRE |
| | LEVKYASLTDLVNNQSVMITLLEEQCLRIFSRQDTHVSPPVLQVVPQHIPNSQQYTPG |
| | LLGGNEIQRDPGYPRDLMPPPDLATSPTKSPFKIPPVTFINEGPFKDCQQAKEAGHSV |
| | SGIYMIKPENSNGPMQLWCENSLDPGGWTVIQKRTDGSVNFFRNWENYKKGFGNIDGE |
| | YWLGLENIYMLSNQDNYKLLIELEDWSDKKVYAEYSSFRLEPESEFYRLRLGTYQGNA |
| | GDSMMWHNGKQFTTLDRDKDMYAGNCAHFHKGGWWYNACAHSNLNGVWYRGGHYRSKH |
| | QDGIFWAEYRGGSYSLRAVQMMIKPIDGGGGGGGGDKTHTCPPCPAPELLGGPSVFLF |
| | PPKPKDTLMISRTPEVTCVVVDVSHEDPEVKFNWYVDGVEVHNAKTKPREEQYNSTYR |
| | VVSVLTVLHQDWLNGKEYKCKVSNKALPAPIEKTISKAKGQPREPQVYTLPPSRDELT |
| | KNQVSLTCLVKGFYPSDIAVEWESNGQPENNYKTTPPVLDSDGSFFLYSKLTVDKSRW |
| | QQGNVFSCSVMHEALHNHYTQKSLSLSPGK |

| | |
|---|---|
| SEQ ID NO:21 | 2118 bp |

| | |
|---|---|
| NOV1k, | AGAGGTGGACAATTCAAAATTAAAAAAATAAACCAGAGAAGATACCCTCGTGCCACAG |
| CG57067-10 | |
| DNA Sequence | ATGGTAAAGAGGAAGCAAAGAAATGTGCATACACATTCCTGGTACCTGAACAAAGAAT |
| | AACAGGGCCAATCTGTGTCAACACCAAGGGQCAAGATGCAAGTACCATTAAAGACATG |
| | ATCACCAGGATGGACCTTGAAAACCTGAAGGATGTGCTCTCCAGGCAGAAGCGGGAGA |
| | TAGATGTTCTGCAACTGGTGGTGGATGTAGATGGAAACATTGTGAATGAGGTAAAGCT |
| | GCTGAGAAAGGAAAGCCGTAACATGAACTCTCGTGTTACTCAACTCTATATGCAATTA |
| | TTACATGAGATTATCCGTAAGAGGGATAATTCACTTGAACTTTCCCAACTGGAAAACA |
| | AAATCCTCAATGTCACCACAGAAATGTTGAAGATGGCAACAAGATACAGGGAACTAGA |
| | GGTGAAATACGCTTCCTTGACTGATCTTGTCAATAACCAATCTGTGATGATCACTTTG |
| | TTGGAAGAACAGTGCTTGAGGATATTTTCCCGACAAGACACCCATGTGTCTCCCCCAC |
| | TTGTCCAGGTGGTGCCACAACATATTCCTAACAGCCAACAGTATACTCCTGGTCTGCT |
| | GGGAGGTAACGAGATTCAGAGGGATCCAGGTTATCCCAGAGATTTAATGCCACCACCT |
| | GATCTGGCAACTTCTCCCACCAAAAGCCCTTTCAAGATACCACCGGTAACTTTCATCA |
| | ATGAAGGACCATTCAAAGACTGTCAGCAAGCAAAAGAAGCTGGGCATTCGGTCAGTGG |
| | GATTTATATGATTAAACCTGAAAACAGCAATGGACCAATGCAGTTATGGTGTGAAAAC |
| | AGTTTGGACCCTGGGGGTTGGACTGTTATTCAGAAAAGAACAGACGGCTCTGTCAACT |
| | TCTTCAGAAATTGGGAAAATTATAAGAAAGGGTTTGGAAACATTGACGGAGAATACTG |
| | GCTTGGACTGGAAAATATCTATATGCTTAGCAATCAAGATAATTACAAGTTATTGATT |
| | GAATTAGAAGACTGGAGTGATAAAAAAGTCTATGCAGAATACAGCAGCTTTCGTCTGG |
| | AACCTGAAAGTGAATTCTATAGACTGCGCCTGGGAACTTACCAGGGAAATGCAGGGGA |
| | TTCTATGATGTGGCATAATGGTAAACAATTCACCACACTGGACAGAGATAAAGATATG |
| | TATGCAGGAAACTGCQCCCACTTTCATAAAGGAGGCTGGTGGTACAATGCCTGTGCAC |
| | ATTCTAACCTAAATGGAGTATGGTACAGAGGAGGCCATTACAGAAGCAAGCACCAAGA |
| | TGGAATTTTCTGGGCCGAATACAGAGGCGGGTCATACTCCTTAAGAGCAGTTCAGATG |
| | ATGATCAAGCCTATTGACGGCGGCGGCGGCGGCGGCGGCGGCGACAJAAACTCACACAT |
| | GCCCACCGTGCCCAGCACCTGAACTCCTGGGGGGACCGTCAGTCTTCCTCTTCCCCCC |
| | AAAACCCAAGGACACCCTCATGATCTCCCGGACCCCTGAGGTCACATGCGTGGTGGTG |
| | GACGTGAGCCACGAAGACCCTGAGGTCAAGTTCAACTGGTACGTGGACGGCGTGGAGG |
| | TGCATAATGCCAAGACAAAGCCGCGGGAGGAGCAGTACAACAGCACGTACCGTGTGGT |
| | CAGCGTCCTCACCGTCCTGCACCAGGACTGGCTGAATGGCAAGGAGTACAAGTGCAAG |
| | GTCTCCAACAAAGCCCTCCCAGCCCCCATCGAGAAAACCATCTCCAAAGCCAAAGGGC |
| | AGCCCCOAGAACCACAGGTGTACACCCTGCCCCCATCCCGGGATGAGCTGACCAAGAA |
| | CCAGGTCAGCCTGACCTGCCTGGTCAAAGGCTTCTATCCCAGCGACATCGCCGTGGAG |
| | TGGGAGAGCAATGGGCAGCCGGAGAACAACTACAAGACCACGCCTCCCGTGCTGGACT |
| | CCGACGGCTCCTTCTTCCTCTACAGCAAGCTCACCGTGGACAAGAGCAGGTGGCAGCA |
| | GGGGAACGTCTTCTCATGCTCCGTGATGCATGAGGCTCTGCACAACCACTACACGCAG |
| | AAGAGCCTCTCCCTGTCTCCGGGTAAATGA |

| | |
|---|---|
| ORF Start: at 1 | ORF Stop: TGA at 2116 |

| | | |
|---|---|---|
| SEQ ID NO: 22 | 705 aa | MW at 80293.5 kD |

| | |
|---|---|
| NOV1k, | RGGQFKIKKINQRRYPRATDGKEEAKKCAYTFLVPEQRITGPICVNTKGQDASTIKDM |
| CG57067-10 | |
| Protein | ITRMDLENLKDVLSRQKREIDVLQLVVDVDGNIVNEVKLLRKESRNMNSRVTQLYMQL |
| Sequence | |
| | LHEIIRKRDNSLELSQLENKILNVTTEMLKMATRYRELEVKYASLTDLVNNQSVMTTL |
| | LEEQCLRIFSRQDThVSPPLVQVVPQHIPNSQQYTPGLLGGNEIQRDPGYPRDLMPPP |
| | DLATSPTKSPFKIPPVTFINEGPFKDCQQAKEAGHSVSGIYMIKPENSNGPMQLWCEN |
| | SLDPGGWTVIQKRTDGSVNFFRNWENYKKGFGNIDGEYWLGLENIYMLSNQDNYKLLI |
| | ELEDWSDKKVYAEYSSFRLEPESEFYRLRLGTYQGNAGDSMMWHNGKQFTTLDRDKDM |
| | YAGNCAHFHKGGWWYNACAHSNLNGVWYRGGHYRSKHQDGIFWAEYRGGSYSLRAVQM |
| | MIKPIDGGGGOGGGDKTHTCPPCPAPELLGGPSVFLFPPKPKDTLMISRTPEVTCVVV |
| | DVSHEDPEVKFNWYVDGVEVHNAKTKPREEQYNSTYRVVSVLTVLHQDWLNGKEYKCK |
| | VSNKALPAPIEKTISKAKGQPREPQVYTLPPSRDELTKNQVSLTCLVKGFYPSDIAVE |
| | WESNGQPENNYKTTPPVLDSDGSFFLYSKLTVDKSRWQQGNVFSCSVMHEALHNHYTQ |
| | KSLSLSPGK |

| | |
|---|---|
| SEQ ID NO:23 | 1947bp |

| | |
|---|---|
| NOV1l, | ATGATCACCAGGATGGACCTTGAAAACCTGAAGGATGTGCTCTCCAGGCAGAAGCGGG |
| CG57067-11 | |
| DNA Sequence | AGATAGATGTTCTGCAACTGGTGGTGGATGTAGATGGAAACATTGTGAATGAGGTAAA |
| | GCTGCTGAGAAAGGAAAGCCGTAACATGAACTCTCGTGTTACTCAACTCTATATGCAA |
| | TTATTACATGAGATTATCCGTAAGAGGGATAATTCACTTGAACTTTCCCAACTGGAAA |
| | ACAAAATCCTCAATGTCACCACAGAAATGTTGAAGATGGCAACAAGATACAGGGAACT |
| | AGAGGTGAAATACGCTTCCTTGACTGATCTTGTCAATAACCAATCTGTGATGATCACT |
| | TTGTTGGAAGAACAGTGCTTGAGGATATTTTCCCGACAAGACACCCATGTGTCTCCCC |
| | CACTTGTCCAGGTGGTGCCACAACATATTCCTAACAGCCAACAGTATACTCCTGGTCT |
| | GCTGGGAGGTAACGAGATTCAGAGGGATCCAGGTTATCCCAGAGATTTAATGCCACCA |
| | CCTGATCTGGCAACTTCTCCCACCAAAAGCCCTTTCAAGATACCACCGGTAACTTTCA |
| | TCAATGAAGGACCATTCAAAGACTGTCAGCAAGCAAAAGAAGCTGGGCATTCGGTCAG |
| | TGGGATTTATATGATTAAACCTGAAAACAGCAATGGACCAATGCAGTTATGGTGTGAA |
| | AACAGTTTGGACCCTGGGGGTTGCACTGTTATTCAGAAAAGAACAGACGGCTCTGTCA |
| | ACTTCTTCAGAAATTGGGAAAATTATAAGAAAGGGTTTGGAAACATTGACGCAGAATA |
| | CTGGCTTGGACTGGAAAATATCTATATGCTTAGCAATCAAGATAATTACAAGTTATTG |
| | ATTGAATTAGAAGACTGGAGTGATAAAAAAGTCTATGCAGAATACAGCAGCTTTCGTC |
| | TGGAACCTGAAAGTGAATTCTATAGACTGCGCCTGGGAACTTACCAGGGAAATGCAGG |
| | GGATTCTATGATGTGGCATAATGGTAAACAATTCACCACACTGGACAGAGATAAAGAT |
| | ATGTATGCAGGAAACTGCGCCCACTTTCATAAAGGAGGCTGGTGGTACAATGCCTGTG |
| | CACATTCTAACCTAAATGGAGTATGGTACAGAGGAGGCCATTACAGAAGCAAGCACCA |
| | AGATGGAATTTTCTGGGCCGAATACAGAGGCGGGTCATACTCCTTAAGAGCAGTTCAG |
| | ATGATGATCAAGCCTATTGACGGCGGCGGCGGCGGCGGCGGCGGCGACAAAACTCACA |
| | CATGCCCACCGTGCCCAGCACCTGAACTCCTGGGGGGACCGTCAGTCTTCCTCTTCCC |
| | CCCAAAACCCAAGGACACCCTCATGATCTCCCGGACCCCTGAGGTCACATGCGTGGTG |
| | GTGGACGTGAGCCACGAAGACCCTGAGGTCAAGTTCAACTGGTACGTGGACGGCGTGG |
| | AGGTGCATAATGCCAAGACAAAGCCGCGGGAGGAGCAGTACAACAGCACQTACCQTGT |
| | GGTCAGCGTCCTCACCGTCCTGCACCAGGACTGGCTGAATGGCAAGGAGTACAAGTGC |
| | AAGGTCTCCAACAAAGCCCTCCCAGCCCCCATCGAGAAAACCATCTCCAAAGCCAAAG |
| | GGCAGCCCCGAGAACCACAGGTGTACACCCTGCCCCCATCCCGGGATGAGCTGACCAA |
| | GAACCAGGTCAGCCTGACCTGCCTGGTCAAAGGCTTCTATCCCAGCQACATCGCCGTG |
| | GAGTGGGAGAGCAATGGGCAGCCGGAGAACAACTACAAGACCACGCCTCCCGTGCTGG |
| | ACTCCGACGGCTCCTTCTTCCTCTACAGCAAGCTCACCGTGGACAAGAGCAGGTGGCA |
| | GCAGGGGAACGTCTTCTCATGCTCCGTGATGCATGAGGCTCTGCACAACCACTACACG |
| | CAGAAGAGCCTCTCCCTGTCTCCGGGTAAATGA |

| | |
|---|---|
| ORF Start: ATG at 1 | ORF Stop: TGA at 1946 |

| | | |
|---|---|---|
| SEQ ID NO:24 | 648 aa | MW at 73856.1 kD |

| | |
|---|---|
| NOV1l, | MITRMDLENLKDVLSRQKREIDVLQLVVDVDGNIVNEVKLLRKESRNMNSRVTQLYMQ |
| CG57067-11 | |
| Protein | LLHEIIRKRDNSLELSQLENKILNVTTEMLKMATRYRELEVKYASLTDLVNNQSVMIT |
| Sequence | |
| | LLEEQCLRIFSRQDTHVSPPLVQVVPQHIPNSQQYTPGLLGGNEIQRDPGYPRDLMPP |
| | PDLATSPTKSPFKIPPVTFINEGPFKDCQQAKEAGHSVSGIYMIKPENSNGPMQLWCE |
| | NSLDPGGWTVIQKRTDGSVNFFRNWENYKKGFGNIDGEYWLGLENIYMLSNQDNYKLL |
| | IELEDWSDKKVYAEYSSFRLEPESEFYRLRLGTYQGNAGDSMMWHNGKQFTTLDRDKD |
| | MYAGNCAHFHKGGWWYNACAHSHLNGVWYRGGHYRSKHQDGIFWAEYRGGSYSLRAVQ |
| | MMIKPIDGGGGGGGGDKTHTCPPCPAPELLGGPSVFLFPPKPKDTLMISRTPEVTCVV |
| | VDVSHEDPEVKFNWYVDGVEVHNAKTKPREEQYNSTYRVVSVLTVLHQDWLNGKEYKC |
| | KVSNKALPAPIEKTISKAKGQPREPQVYTLPPSRDELTKNQVSLTCLVKGFYPSDIAV |
| | EWESNGQPENNYKTTPPVLDSDGSFFLYSKLTVDKSRWQQGNVFSCSVMHEALHNHYT |
| | QKSLSLSPGK |

| | |
|---|---|
| SEQ ID NO: 25 | 1998 bp |

| | |
|---|---|
| NOV1m, | GGGCCAATCTGTGTCAACACCAAGGGGCAAGATGCAAGTACCATTAAAGACATGATCA |
| CG57067-12 | |
| DNA Sequence | CCAGGATGGACCTTGAAAACCTGAAGGATGTGCTCTCCAGGCAGAAGCGGGAGATAGA |
| | TGTTCTGCAACTGGTGGTGGATGTAGATGGAAACATTGTGAATGAGGTAAAGCTGCTG |
| | AGAAAGGAAAGCCGTAACATGAACTCTCGTGTTACTCAACTCTATATGCAATTATTAC |
| | ATGAGATTATCCGTAAGAGGGATAATTCACTTGAACTTTCCCAACTGGAAAACAAAAT |
| | CCTCAATGTCACCACAGAAATGTTGAAGATGGCAACAAGATACAGGGAACTAGAGGTG |
| | AAATACGCTTCCTTGACTGATCTTGTCAATAACCAATCTGTGATGATCACTTTGTTGG |
| | AAGAACAGTGCTTGAGGATATTTTCCCGACAAGACACCCATGTGTCTCCCCCACTTGT |
| | CCAGGTGGTGCCACAACATATTCCTAACAGCCAACAGTATACTCCTGGTCTGCTGGGA |
| | GGTAACGAGATTCAGAGGGATCCAGGTTATCCCAGAGATTTAATGCCACCACCTGATC |
| | TGGCAACTTCTCCCACCAAAAGCCCTTTCAAGATACCACCGGTAACTTTCATCAATGA |
| | AGGACCATTCAAAGACTGTCAGCAAGCAAAAGAAGCTGGGCATTCGGTCAGTQGGATT |
| | TATATGATTAAACCTGAAAACACCAATGGACCAATGCAGTTATGGTGTGAAAACAGTT |
| | TGGACCCTGGGGGTTGGACTGTTATTCAGAAAAGAACAGACGGCTCTGTCAACTTCTT |
| | CAGAAATTGGGAAAATTATAAQAAAGGGTTTGGAAACATTGACCGAGAATACTGGCTT |
| | GGACTGGAAAATATCTATATGCTTAGCAATCAAGATAATTACAAGTTATTGATTGAAT |
| | TAGAAGACTGGAGTGATAAAAAAGTCTATGCAGAATACAGCAGCTTTCGTCTGGAACC |
| | TGAAAGTGAATTCTATAGACTGCGCCTGGGAACTTACCAGGGAAATGCAGGGGATTCT |
| | ATGATGTGGCATAATGGTAAACAATTCACCACACTGGACAGAGATAAAGATATGTATG |
| | CAGGAAACTGCGCCCACTTTCATAAAGGAGGCTGGTGGTACAATGCCTGTGCACATTC |
| | TAACCTAAATGGAGTATGGTACAGAGGAGGCCATTACAGAAGCAAGCACCAAGATGGA |
| | ATTTTCTGCGCCGAATACAGAGGCGGGTCATACTCCTTAAGAGCAGTTCAGATGATGA |
| | TCAAGCCTATTGACGGCGGCGGCGGCGGCGGCGGCGGCGACAAAACTCACACATGCCC |
| | ACCGTGCCCAGCACCTGAACTCCTGGGGGGACCGTCAGTCTTCCTCTTCCCCCCAAAA |
| | CCCAAGGACACCCTCATGATCTCCCGGACCCCTGAGGTCACATGCGTGGTGGTGGACG |
| | TGAGCCACGAAGACCCTGAGGTCAAGTTCAACTGGTACGTGGACGGCGTGGAQGTGCA |
| | TAATGCCAAGACAAAGCCGCGGGAOGAGCAGTACAACAGCACGTACCGTGTGGTCAGC |
| | GTCCTCACCGTCCTGCACCAGGACTGGCTGAATGGCAAGGAGTACAAGTGCAAGGTCT |
| | CCAACAAAGCCCTCCCAGCCCCCATCGAGAAAACCATCTCCAAAGCCAAAGGGCAGCC |
| | CCGAGAACCACAGGTGTACACCCTGCCCCCATCCCGGGATGAGCTGACCAAGAACCAG |
| | GTCAGCCTGACCTGCCTGGTCAAAGGCTTCTATCCCAGCGACATCGCCGTGGAGTGGG |
| | AGAGCAATGGGCAGCCGGAGAACAACTACAAGACCACGCCTCCCGTGCTGGACTCCGA |
| | CGGCTCCTTCTTCCTCTACAGCAAGCTCACCGTGGACAAGAGCAGGTGGCAGCAGGGG |
| | AACGTCTTCTCATGCTCCOTGATGCATGAGGCTCTGCACAACCACTACACGCAGAAGA |
| | GCCTCTCCCTGTCTCCGGGTAAATGA |

| | |
|---|---|
| ORF Start: at 1 | ORF Stop: TGA at 1996 |

| | | |
|---|---|---|
| SEQ ID NO:26 | 665 aa | MW at 75585.0 kD |

| | |
|---|---|
| NOV1m, | GPICVNTKGQDASTIKDMITRMDLENLKDVLSRQKREIDVLQLVVDVDGNIVNEVKLL |
| CG57067-12 | |
| Protein | RKESRNMNSRVTQLYMQLLHEIIRKRDNSLELSQLENKILNVTTEMLKMATRYRELEV |
| Sequence | |
| | KYASLTDLVNNQSVMITLLEEQCLRIFSRQDTHVSPPLVQVVPQHIPNSQQYTPGLLG |
| | GNEIQRDPGYPRDLMPPPDLATSPTKSPFKIPPVTFDIEGPFKDCQQAKEAGHSVSGI |
| | YMIKPENSNGPMQLWCENSLDPGGWTVIQKRTDGSVNFFRNWENYKKGFGNIDGEYWL |
| | GLENIYMLSNQDNYKLLIELEDWSDKKVYAEYSSFRLEPESEFYRLRLGTYQGNAGDS |
| | MMWHNGKQFTTLDRDKDMYAGNCAHFHKGGWWYNACAHSNLNGVWYRGGHYRSKHQDG |
| | IFWAEYRGGSYSLRAVQMMIKPIDGGGGGGGGDKTHTCPPCPAPELLGGPSVFLFPPK |
| | PKDTLMISRTPEVTCVVVDVSHEDPEVKFNWYVDGVEVHNAKTKPREEQYNSTYRVVS |
| | VLTVLHQDWLNGKEYKCKVSNKALPAPIEKTISKAKGQPREPQVYTLPPSRDELTKNQ |
| | VSLTCLVKGFYPSDIAVEWESNGQPENNYKTTPPVLDSDGSFFLYSKLTVDKSRWQQG |
| | NVFSCSVMHEALHNHYTQKSLSLSPGK |

| | |
|---|---|
| SEQ ID NO:27 | 1767 bp |

| | |
|---|---|
| NOV1n, | CATGAGATTATCCGTAAGAGGGATAATTCACTTGAACTTTCCCAACTGGAAAACAAA |
| CG57067-13 | |
| DNA Sequence | TCCTCAATGTCACCACAGAAATGTTGAAGATGGCAACAAGATACAGGGAACTAGAGGT |
| | GAAATACGCTTCCTTGACTGATCTTGTCAATAACCAATCTGTCATGATCACTTTGTTG |
| | GAAGAACAGTGCTTGAGGATATTTTCCCGACAAGACACCCATGTGTCTCCCCCACTTG |
| | TCCAGGTGGTGCCACAACATATTCCTAACAGCCAACAGTATACTCCTGGTCTGCTGGG |
| | AGGTAACGAGATTCAGAGGGATCCAGGTTATCCCAGAGATTTAATGCCACCACCTGAT |
| | CTGGCAACTTCTCCCACCAAAAGCCCTTTCAAGATACCACCGGTAACTTTCATCAATG |
| | AAGGACCATTCAAAGACTGTCAGCAAGCAAAAGAAGCTGGGCATTCCGTCAQTGGGAT |
| | TTATATGATTAAACCTGAAAACACCAATGGACCAATGCAGTTATGGTGTGAAAACAGT |
| | TTGGACCCTQGGGGTTGGACTGTTATTCAGAAAAGAACAGACGGCTCTGTCAACTTCT |
| | TCAGAAATTGGGAAAATTATAAGAAAGGGTTTGGAAACATTGACGGAGAATACTGGCT |
| | TGGACTGGAAAATATCTATATGCTTAGCAATCAAGATAATTACAAGTTATTGATTGAA |
| | TTAGAAGACTGGAGTGATAAAAAAGTCTATGCAGAATACACCAGCTTTCGTCTGGAAC |
| | CTGAAAGTGAATTCTATAGACTGCGCCTGGGAACTTACCAGGGAAATGCAGGGGATTC |
| | TATGATGTGGCATAATGGTAAACAATTCACCACACTGGACAGAGATAAAGATATGTAT |
| | GCAGGAAACTGCGCCCACTTTCATAAAGGAGGCTGGTGGTACAATGCCTGTGCACATT |
| | CTAACCTAAATGGAGTATGGTACAGAGGAGGCCATTACAGAAGCAAGCACCAAGATGG |
| | AATTTTCTGGGCCGAATACAGAGGCGGGTCATACTCCTTAAGAGCAGTTCACATGATG |
| | ATCAAGCCTATTGACGGCGGCGGCGGCGGCGGCGGCGGCGACAAAACTCACACATGCC |
| | CACCGTGCCCAGCACCTGAACTCCTGGGGGGACCGTCAGTCTTCCTCTTCCCCCCAAA |
| | ACCCAACGACACCCTCATGATCTCCCGGACCCCTGAGGTCACATGCGTGGTGGTGGAC |
| | GTGAGCCACGAAGACCCTGAGGTCAAGTTCAACTCGTACGTGGACGGCGTGGAGGTGC |
| | ATAATGCCAAGACAAAGCCGCGGGAGGAGCAGTACAACAGCACGTACCGTGTGGTCAG |
| | CGTCCTCACCGTCCTGCACCAGGACTGGCTGAATGGCAAGGAGTACAAGTGCAAGGTC |
| | TCCAACAAAGCCCTCCCAGCCCCCATCGAGAAAACCATCTCCAAAGCCAAAGGGCAGC |
| | CCCGAGAACCACAGGTGTACACCCTGCCCCCATCCCGGGATGAGCTGACCAAGAACCA |
| | GGTCAGCCTGACCTGCCTGGTCAAAGGCTTCTATCCCAGCGACATCGCCGTGGAGTGG |
| | GAGAGCAATGGGCAGCCGGAGAACAACTACAAGACCACGCCTCCCGTGCTGGACTCCG |
| | ACGGCTCCTTCTTCCTCTACAGCAAGCTCACCGTGGACAAGAGCAGGTGQCAGCAGGG |
| | GAACGTCTTCTCATGCTCCGTGATGCATGAGGCTCTGCACAACCACTACACGCAGAAG |
| | AGCCTCTCCCTGTCTCCGGGTAAATGA |

| | |
|---|---|
| ORF Start: at 1 | ORF Stop: TGA at 1765 |

| | | |
|---|---|---|
| SEQ ID NO: 28 | 588 aa | MW at 6675.8 kD |

| | |
|---|---|
| NOV1n, | HEIIRKRDNSLELSQLENKILNVTTEMLKMATRYRELEVKYASLTDLVNNQSVMITLL |
| CG57067-13 | |
| Protein | EEQCLRIFSRQDTHVSPPLVQVVPQHIPNSQQYTPGLLGGNEIQRDPGYPRDLMPPPD |
| Sequence | |
| | LATSPTKSPFKIPPVTFINEGPFKDCQQAKEAGHSVSGIYMIKPENSNGPMQLWCENS |
| | LDPGGWTVIQKRTDGSVNFFRNWENYKKGFGNIDGEYWLGLENIYMLSNQDNYKLLIE |
| | LEDWSDKKVYAEYSSFRLEPESEFYRLRLGTYQGNAGDSMMWHNGKQFTTLDRDKDMY |
| | AGNCAHFHKGGWWYNACAHSNLNGVWYRGGHYRSKHQDGIFWAEYRGGSYSLPAVQMM |
| | IKPIDGGGGGGGGDKTHTCPPCPAPELLGGPSVFLFPPKPKDTLMISRTPEVTCVVVD |
| | VSHEDPEVKFNWYVDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGFYPSDIAVEW |
| | ESNGQPENNYKTTPPVLDSDGSFFLYSKLTVDKSRWQQGNVFSCSVMHEALHNHYTQK |

| | |
|---|---|
| SEQ ID NO: 29 | 1470 bp |

| | |
|---|---|
| NOV1o, | GAGATTCAGAGGGATCCAGGTTATCCCAGAGATTTAATGCCACCACCTGATCTGGCAA |
| GG57067-14 | |
| DNA Sequence | CTTCTCCCACCAAAAGCCCTTTCAAGATACCACCGGTAACTTTCATCAATGAAGGACC |
| | ATTCAAAGACTGTCAGCAAGCAAAAGAAGCTGGGCATTCGGTCAGTGGGATTTATATG |
| | ATTAAACCTGAAAACAGCAATGGACCAATGCAGTTATGGTGTGAAAACAGTTTGGACC |
| | CTGGGGGTTGGACTGTTATTCAGAAAAGAACAGACGGCTCTGTCAACTTCTTCAGAAA |
| | TTGGGAAAATTATAAGAAAGGGTTTGGAAACATTGACGGAGAATACTGGCTTGGACTG |
| | GAAAATATCTATATGCTTAGCAATCAAGATAATTACAAGTTATTGATTGAATTAGAAG |
| | ACTGGAGTGATAAAAAAGTCTATGCAGAATACAGCAGCTTTCGTCTGGAACCTGAAAG |
| | TGAATTCTATAGACTGCGCCTGGGAACTTACCAGGGAAATGCAGGGGATTCTATGATG |
| | TGGCATAATGGTAAACAATTCACCACACTGGACAGAGATAAAGATATGTATGCAGGAA |
| | ACTGCGCCCACTTTCATAAAGGAGGCTGGTGGTACAATGCCTGTGCACATTCTAACCT |
| | AAATGGAGTATGGTACAGAGGAGGCCATTACAGAAGCAAGCACCAAGATGGAATTTTC |
| | TGGGCCGAATACAGAGGCGGGTCATACTCCTTAAGAGCAGTTCAGATGATGATCAAGC |
| | CTATTGACGGCGGCGGCGGCGGCGGCGGCGGCGACAAAACTCACACATGCCCACCGTG |
| | CCCAGCACCTGAACTCCTGGGGCGACCGTCAGTCTTCCTCTTCCCCCCAAAACCCAAG |
| | GACACCCTCATGATCTCCCGGACCCCTGAGGTCACATGCGTGGTGGTGGACGTGAGCC |
| | ACGAAGACCCTGAGGTCAAGTTCAACTGGTACGTGGACGGCGTGGAGGTGCATAATGC |
| | CAAGACAAAGCCGCGGGAGGAGCAGTACAACAGCACGTACCGTGTGGTCAGCGTCCTC |
| | ACCGTCCTGCACCAGGACTGGCTGAATGGCAAGGAGTACAAGTGCAAGGTCTCCAACA |
| | AAGCCCTCCCAGCCCCCATCGAGAAAACCATCTCCAAAGCCAAAGGGCAGCCCCGAGA |
| | ACCACAGGTGTACACCCTGCCCCCATCCCGGGATGAGCTGACCAAGAACCAGGTCAGC |
| | CTGACCTGCCTGGTCAAAGGCTTCTATCCCAGCGACATCGCCGTGGAGTGGGAGAGCA |
| | ATGGGCAGCCGGAGAACAACTACAAGACCACGCCTCCCGTGCTGGACTCCGACGGCTC |
| | CTTCTTCCTCTACAGCAAGCTCACCGTGGACAAGAGCAGGTGGCAGCAGGGGAACGTC |
| | TTCTCATGCTCCGTGATGCATGAGGCTCTGCACAACCACTACACGCAGAAGAGCCTCT |
| | CCCTGTCTCCGGGTAAATGA |

| | |
|---|---|
| ORF Start: at 1 | ORF Stop: TGA at 1468 |

| | | |
|---|---|---|
| SEQ ID NO:30 | 489 aa | MW at 55389.9 kD |

| | |
|---|---|
| NOV1o, | EIQRDPGYPRDLMPPPDLATSPTKSPFKIPPVTFINEGPFKDCQQAKEAGHSVSGIYM |
| CG57067-14 | |
| Protein | IKPENSNGPMQLWCENSLDPGGWTVIQKRTDGSVNFFRNWENYKKGFGNIDGEYWLGL |
| Sequence | |
| | ENIYMLSNQDNYKLLIELEDWSDKKVYAEYSSFRLEPESEFYRLRLGTYQGNAGDSMM |
| | WHNGKQFTTLDRDKDMYAGNCAHFHKGGWWYNACAHSNLNGVWYRGGHYRSKHQDGIF |
| | WAEYRGGSYSLRAVQMMIKPIDGGGGGGGGDKTHTCPPCPAPELLGGPSVFLFPPKPK |
| | DTLMISRTPEVTCVVVDVSHEDPEVKFNWYVDGVEVHNAKTKPREEQYNSTYRVVSVL |
| | TVLHQDWLNGKEYKCKVSNKALPAPIEKTISKAKGQPREPQVYTLPPSRDELTKNQVS |
| | LTCLVKGFYPSDIAVEWESNGQPENNYKTTPPVLDSDGSFFLYSKLTVDKSRWQQGNV |
| | FSCSVMHEALHNHYTQKSLSLSPGK |

| | |
|---|---|
| SEQ ID NO: 31 | 1389 bp |

| | |
|---|---|
| NOV1p | AAGATACCACCGGTAACTTTCATCAATGAAGGACCATTCAAAGACTGTCAGCAAGCAA |
| CG57067-15 | |
| DNA Sequence | AAGAAGCTGGGCATTCGGTCAGTGGGATTTATATGATTAAACCTGAAAACAGCAATGG |
| | ACCAATGCAGTTATGGTGTGAAAACAGTTTGGACCCTGGGGGTTGGACTGTTATTCAG |
| | AAAAGAACAGACGGCTCTGTCAACTTCTTCAGAAATTGGGAAAATTATAAGAAAGGGT |
| | TTGGAAACATTGACGGAGAATACTGGCTTGGACTGGAAAATATCTATATGCTTAGCAA |
| | TCAAGATAATTACAAGTTATTGATTGAATTAGAAGACTGGACTGATAAAAAAGTCTAT |
| | GCAGAATACAGCAGCTTTCGTCTGGAACCTGAAAGTGAATTCTATAGACTGCGCCTGG |
| | GAACTTACCAGGGAAATGCAGGGGATTCTATGATGTGGCATAATGGTAAACAATTCAC |
| | CACACTGGACAGAGATAAAGATATGTATGCAGGAAACTGCGCCCACTTTCATAAAGGA |
| | GGCTGGTGGTACAATGCCTGTGCACATTCTAACCTAAATGGAGTATGGTACAGAGGAG |
| | GCCATTACAGAAGCAAGCACCAAGATGGAATTTTCTGGGCCGAATACAGAGGCGGGTC |
| | ATACTCCTTAAGAGCAGTTCAGATGATGATCAAGCCTATTGACGGCGGCGGCGGCGGC |
| | GGCGGCGGCGACAAAACTCACACATGCCCACCGTGCCCAGCACCTGAACTCCTGGGGG |
| | GACCGTCAGTCTTCCTCTTCCCCCCAAAACCCAAGGACACCCTCATGATCTCCCGGAC |
| | CCCTGAGGTCACATGCGTGGTGGTGGACGTGAGCCACGAAGACCCTGAGGTCAAGTTC |
| | AACTGGTACGTGGACGGCGTGGAGGTGCATAATGCCAAGACAAAGCCGCGGGAGGAGC |
| | AGTACAACAGCACGTACCGTGTGGTCAGCGTCCTCACCGTCCTGCACCAGGACTGGCT |
| | GAATGGCAAGGAGTACAAGTGCAAGGTCTCCAACAAAGCCCTCCCAGCCCCCATCGAG |
| | AAAACCATCTCCAAAGCCAAAGGGCAGCCCCGAGAACCACAGGTGTACACCCTGCCCC |
| | CATCCCGGGATGAGCTGACCAAGAACCAGGTCAGCCTGACCTGCCTGGTCAAAGGCTT |
| | CTATCCCAGCGACATCGCCGTGGAGTGGGAGAGCAATGGGCAGCCGGAGAACAACTAC |
| | AAGACCACGCCTCCCGTGCTGGACTCCGACGGCTCCTTCTTCCTCTACAGCAAGCTCA |
| | CCGTGGACAAGAGCAGGTGGCAGCAGGGGAACGTCTTCTCATGCTCCGTGATGCATGA |
| | GGCTCTGCACAACCACTACACGCAGAAGAGCCTCTCCCTGTCTCCGGGTAAATGA |

| | |
|---|---|
| ORF Start: at 1 | ORF Stop: TGA at 1387 |

| | | |
|---|---|---|
| SEQ ID NO:32 | 462 aa | MW at 52381.5 kD |

| | |
|---|---|
| NOV1p, | KIPPVTFINEGPFKDCQQAKEAGHSVSGIYMIKPENSNGPMQLWCENSLDPGGWTVIQ |
| CG57067-15 | |
| Protein | KRTDGSVNFFRNWENYKKGFGNIDGEYWLGLENIYMLSNQDNYKLLLELEDWSDKKVY |
| Sequence | |
| | AEYSSFRLEPESEFYRLRLGTYQGNAGDSMMWHNGKQFTTLDRDKDMYAGNCAHFHKG |
| | GWWYNACAHSNLNGVWYRGGHYRSKHQDGIFWAEYRGGSYSLRAVQMMIKPIDGGGGG |
| | GGGDKTHTCPPCPAPELLGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALPAPIE |
| | KTISKAKGQPREPQVYTLPPSRDELTKNQVSLTCLVKGFYPSDIAVEWESNGQPENNY |
| | KTTPPVLDSDGSFFLYSKLTVDKSRWQQGNVFSCSVMHEALHNHYTQKSLSLSPGK |

| | |
|---|---|
| SEQ ID NO:33 | 2118 bp |

| | |
|---|---|
| NOV1q, | GACAAAACTCACACATGCCCACCGTGCCCAGCACCTGAACTCCTGGGGGGACCGTCAG |
| CG57067-16 | |
| DNA Sequence | TCTTCCTCTTCCCCCCAAAACCCAAGGACACCCTCATGATCTCCCGGACCCCTGAGGT |
| | CACATGCGTGGTGGTGGACGTGAGCCACGAAGACCCTGAGGTCAAGTTCAACTGGTAC |
| | GTGGACGGCGTGGAGGTGCATAATGCCAAGACAAAGCCGCGGGAGGAGCAGTACAACA |
| | GCACGTACCGTGTGGTCAGCGTCCTCACCGTCCTGCACCAGGACTGGCTGAATGGCAA |
| | GGAGTACAAGTGCAAGGTCTCCAACAAAGCCCTCCCAGCCCCCATCGAGAAAACCATC |
| | TCCAAAGCCAAAGGGCAGCCCCGAGAACCACAGGTGTACACCCTGCCCCCATCCCGGG |
| | ATGAGCTGACCAAGAACCAGGTCAGCCTGACCTGCCTGGTCAAAGGCTTCTATCCCAG |
| | CGACATCGCCGTGGAGTGGGAGAGCAATGGGCAGCCGGAGAACAACTACAAGACCACG |
| | CCTCCCGTGCTGGACTCCGACGGCTCCTTCTTCCTCTACAGCAAGCTCACCGTGGACA |
| | AGAGCAGGTGGCAGCAGGGGAACGTCTTCTCATGCTCCGTGATGCATGAGGCTCTGCA |
| | CAACCACTACACGCAGAAGAGCCTCTCCCTGTCTCCGGGTAAAGGCGGCGGCGGCGGC |
| | GGCGGCGGCAGAGGTGGACAATTCAAAATTAAAAAAATAAACCAGAGAAGATACCCTC |
| | GTGCCACAGATGGTAAAGAGGAAGCAAAGAAATGTGCATACACATTCCTGGTACCTGA |
| | ACAAAGAATAACAGGGCCAATCTGTGTCAACACCAAGGGGCAAGATGCAAGTACCATT |
| | AAAGACATGATCACCAGGATGGACCTTGAAAACCTGAAGGATGTGCTCTCCAGGCAGA |
| | AGCGGGAGATAGATGTTCTGCAACTGGTGGTGGATGTAGATGGAAACATTGTGAATGA |
| | GGTAAAGCTGCTGAGAAAGGAAAGCCGTAACATGAACTCTCGTGTTACTCAACTCTAT |
| | ATGCAATTATTACATGAGATTATCCGTAAGAGGGATAATTCACTTGAACTTTCCCAAC |
| | TGGAAAACAAAATCCTCAATGTCACCACAGAAATGTTGAAGATGGCAACAAGATACAG |
| | GGAACTAGAGGTGAAATACGCTTCCTTGACTGATCTTGTCAATAACCAATCTGTGATG |
| | ATCACTTTGTTGGAAGAACAGTGCTTGAGGATATTTTCCCGACAAGACACCCATGTGT |
| | CTCCCCCACTTGTCCAGGTGGTGCCACAACATATTCCTAACAGCCAACAGTATACTCC |
| | TGGTCTGCTGGGAGGTAACGAGATTCAGAGGGATCCAGGTTATCCCAGAGATTTAATG |
| | CCACCACCTGATCTGGCAACTTCTCCCACCAAAAGCCCTTTCAAGATACCACCGGTAA |
| | CTTTCATCAATGAAGGACCATTCAAAGACTGTCAGCAAGCAAAAGAAGCTGGCCATTC |
| | GGTCAGTGGGATTTATATGATTAAACCTGAAAACAGCAATGGACCAATGCAGTTATGG |
| | TGTGAAAACAGTTTGGACCCTGGGGGTTGGACTGTTATTCAGAAAAGAACAGACGGCT |
| | CTGTCAACTTCTTCAGAAATTGGGAAAATTATAAGAAAGGGTTTGGAAACATTGACGG |
| | AGAATACTGGCTTGGACTGGAAAATATCTATATGCTTAGCAATCAAGATAATTACAAG |
| | TTATTGATTGAATTAGAAGACTGGAGTGATAAAAAAGTCTATGCAGAATACAGCAGCT |
| | TTCGTCTGGAACCTGAAAGTGAATTCTATAGACTGCGCCTGGGAACTTACCAGGGAAA |
| | TGCAGGGGATTCTATGATGTGGCATAATGGTAAACAATTCACCACACTGGACAGAGAT |
| | AAAGATATGTATGCAGGAAACTGCGCCCACTTTCATAAAGGAGGCTGGTGGTACAATG |
| | CCTGTGCACATTCTAACCTAAATGGAGTATGGTACAGAGGAGGCCATTACAGAAGCAA |
| | GCACCAAGATGGAATTTTCTGGGCCGAATACAGAGGCGGGTCATACTCCTTAAGAGCA |
| | GTTCAGATGATGATCAAGCCTATTGACTGA |

| | |
|---|---|
| ORF Start: at 1 | ORF Stop: TGA at 2116 |

| | | |
|---|---|---|
| SEQ ID NO: 34 | 705 aa | MW at 80293.5 kD |

| | |
|---|---|
| NOV1q, | DKTHTCPPCPAPELLGGPSVFLFPPKPKDTLMISRTPEVTCVVVDVSHEDPEVKFNWY |
| CG57067-16 | |
| Protein | VDGVEVHNAKTKPREEQYNSTYRVVSVLTVLHQDWLNGKEYKCKVSNKALPAPIEKTI |
| Sequence | |
| | SKAKGQPREPQVYTLPPSRDELTKNQVSLTCLVKGFYPSDIAVEWESNGQPENNYKTT |
| | PPVLDSDGSFFLYSKLTVDKSRWQQGNVFSCSVMHEALHNHYTQKSLSLSPGKGGGGG |
| | GGGRGGQFKIKKINQRRYPRATDGKEEAKKCAYTFLVPEQRITGPICVNTKGQDASTI |
| | KDMITRMDLENLKDVLSRQKREIDVLQLVVDVDGNIVNEVKLLRKESRNMNSRVTQLY |
| | MQLLHETIRKRDNSLELSQLENKILNVTTEMLKMATRYRELEVKYASLTDLVNNQSVM |
| | ITLLEEQCLRIFSRQDTHVSPPLVQVVPQHIPNSQQYTPGLLGGNEIQRDPGYPRDLM |
| | PPPDLATSPTKSPFKIPPVTFINEGPFKDCQQAKEAGHSVSGIYMIKPENSNGPMQLW |
| | CENSLDPGCWTVIQKRTDGSVNFFRNWENYKKGFGNIDGEYWLGLENIYMLSNQDNYK |
| | LLIELEDWSDKKVYAEYSSFRLEPESEFYRLRLGTYQGNAGDSMMWHNGKQFTTLDRD |
| | KDMYAGNCAHFHKGGWWYNACAHSNLNGVWYRGGHYRSKHQDGIFWAEYRGGSYSLRA |
| | VQMMIKPID |

| | |
|---|---|
| SEQ ID NO: 35 | 1947 bp |

| | |
|---|---|
| NOV1r, | GACAAAACTCACACATGCCCACCGTGCCCAGCACCTGAACTCCTGGGGGGACCGTCAG |
| CG57067-17 | |
| DNA Sequence | TCTTCCTCTTCCCCCCAAAACCCAAGGACACCCTCATGATCTCCCGGACCCCTGAGGT |
| | CACATGCGTGGTGGTGGACGTGAGCCACGAAGACCCTGAGGTCAAGTTCAACTGGTAC |
| | GTGGACGGCGTGGAGGTGCATAATGCCAAGACAAAGCCGCGGGAGGAGCAGTACAACA |
| | GCACGTACCGTGTGGTCAGCGTCCTCACCGTCCTGCACCAGGACTGGCTGAATGGCAA |
| | GGAGTACAAGTGCAAGGTCTCCAACAAAGCCCTCCCAGCCCCCATCGAGAAAACCATC |
| | TCCAAAGCCAAAGGGCAGCCCCGAGAACCACAGGTGTACACCCTGCCCCCATCCCGGG |
| | ATGAGCTGACCAAGAACCAGGTCAGCCTGACCTGCCTGGTCAAAGGCTTCTATCCCAG |
| | CGACATCGCCGTGGAGTGGGAGAGCAATGGGCAGCCGGAGAACAACTACAAGACCACG |
| | CCTCCCGTGCTGGACTCCGACGGCTCCTTCTTCCTCTACAGCAAGCTCACCGTGGACA |
| | AGAGCAGGTGGCAGCAGGGGAACGTCTTCTCATGCTCCGTGATGCATGAGGCTCTGCA |
| | CAACCACTACACGCAGAAGAGCCTCTCCCTGTCTCCGGGTAAAGGCGGCGGCGGCGGC |
| | GGCGGCGGCATGATCACCAGGATGGACCTTGAAAACCTGAAGGATGTGCTCTCCAGGC |
| | AGAAGCGGGAGATAGATGTTCTGCAACTGGTGGTGGATGTAGATGGAAACATTGTGAA |
| | TGAGGTAAAGCTGCTGAGAAAGGAAAGCCGTAACATGAACTCTCGTGTTACTCAACTC |
| | TATATGCAATTATTACATGAGATTATCCGTAAGAGGGATAATTCACTTGAACTTTCCC |
| | AACTGGAAAACAAAATCCTCAATGTCACCACAGAAATGTTGAAGATGGCAACAAGATA |
| | CAGGGAACTAGAGGTGAAATACQCTTCCTTGACTGATCTTGTCAATAACCAATCTGTG |
| | ATGATCACTTTGTTGGAAGAACAGTGCTTGAGGATATTTTCCCGACAAGACACCCATG |
| | TGTCTCCCCCACTTGTCCAGGTGGTGCCACAACATATTCCTAACAGCCAACAGTATAC |
| | TCCTGGTCTGCTGGGAGGTAACGAGATTCAGAGGGATCCAGGTTATCCCAGAGATTTA |
| | ATGCCACCACCTGATCTGGCAACTTCTCCCACCAAAAGCCCTTTCAAGATACCACCGG |
| | TAACTTTCATCAATGAAGGACCATTCAAAGACTGTCAGCAAGCAAAAGAAGCTGGGCA |
| | TTCGGTCAGTGGGATTTATATGATTAAACCTGAAAACAGCAATGGACCAATGCAGTTA |
| | TGGTGTGAAAACAGTTTGGACCCTGGGGGTTGGACTGTTATTCAGAAAAGAACAGACG |
| | GCTCTGTCAACTTCTTCAGAAATTGGGAAAATTATAAGAAAGGGTTTGGAAACATTGA |
| | CGGAGAATACTGGCTTGGACTGGAAAATATCTATATGCTTAGCAATCAAGATAATTAC |
| | AAGTTATTGATTGAATTAGAAGACTGGAGTGATAAAAAAGTCTATGCAGAATACAGCA |
| | GCTTTCGTCTGGAACCTGAAAGTGAATTCTATAGACTGCGCCTGGGAACTTACCAGGG |
| | AAATGCAGGGGATTCTATGATGTGGCATAATGGTAAACAATTCACCACACTGGACAGA |
| | GATAAAGATATGTATGCAGGAAACTGCGCCCACTTTCATAAAGGAGGCTGGTGGTACA |
| | ATGCCTGTGCACATTCTAACCTAAATGGAGTATGGTACAGAGGAGGCCATTACAGAAG |
| | CAAGCACCAAGATGGAATTTTCTGGGCCGAATACAGAGGCGGGTCATACTCCTTAAGA |
| | GCAGTTCAGATGATGATCAAGCCTATTGACTGA |

| | |
|---|---|
| ORF Start at 1 | ORF Stop: TGA at 1946 |

| | | |
|---|---|---|
| SEQ ID NO:36 | 648 aa | MW at 73856.1 kD |

| | |
|---|---|
| NOV1r, | DKTHTCPPCPAPELLGGPSVFLFPPKPKDTLMISRTPEVTCVVVDVSHEDPEVKFNWY |
| CG57067-17 | |
| Protein | VDGVEVHNAKTKPREEQYNSTYRVVSVLTVLHQDWLNGKEYKCKVSNKALPAPIEKTI |
| Sequence | |
| | SKAKGQPREPQVYTLPPSRDELTKNQVSLTCLVKGFYPSDIAVEWESNGQPENNYKTT |
| | PPVLDSDGSFFLYSKLTVDKSRWQQGNVFSCSVMHEALHNHYTQKSLSLSPGKGGGGG |
| | GGGMITRMDLENLKDVLSRQKREIDVLQLVVDVDGNIVNEVKLLRKESRNMNSRVTQL |
| | YMQLLHEIIRKRDNSLELSQLENKILNVTTEMLKMATRYRELEVKYASLTDLVNNQSV |
| | MITLLEEQCLRIFSRQDTHVSPPLVQVVPQHIPNSQQYTPGLLGGNEIQRDPGYPRDL |
| | MPPPDLATSPTKSPFKTPPVTFINEGPFKDCQQAKEAGHSVSGIYMIKPENSNGPMQL |
| | WCENSLDPGGWTVIQKRTDGSVNFFRNWENYKKGFGNIDGEYWLGLENIYMLSNQDNY |
| | KLLIELEDWSDKKVYAEYSSFRLEPESEFYRLRLGTYQGNAGDSMMWHNGKQFTTLDR |
| | DKDMYAGNCAHFHKGGWWYNACAHSNLNGVWYRGGHYRSKHQDGIFWAEYRGGSYSLR |
| | AVQMMIKPID |

| | |
|---|---|
| SEQ ID NO:37 | 1998bp |

| | |
|---|---|
| NOV1s, | GACAAAACTCACACATGCCCACCGTGCCCAGCACCTGAACTCCTGGGGGGACCGTCAG |
| CG57067-18 | |
| DNA Sequence | TCTTCCTCTTCCCCCCAAAACCCAAGGACACCCTCATGATCTCCCGGACCCCTGAGGT |
| | CACATGCGTGGTGGTGGACGTGAGCCACGAAGACCCTGAGGTCAAGTTCAACTGGTAC |
| | GTGGACGGCGTGGAGGTGCATAATGCCAAGACAAAGCCGCGGGAGGAGCAGTACAACA |
| | GCACGTACCGTGTGGTCAGCGTCCTCACCGTCCTGCACCAGGACTGGCTGAATGGCAA |
| | GGAGTACAAGTGCAAGGTCTCCAACAAAGCCCTCCCAGCCCCCATCGAGAAAACCATC |
| | TCCAAAGCCAAAGGGCAGCCCCGAGAACCACAGGTGTACACCCTGCCCCCATCCCGcIG |
| | ATGAGCTGACCAAGAACCAGGTCAGCCTGACCTGCCTGGTCAAAGGCTTCTATCCCAG |
| | CGACATCGCCGTGGAGTCGGAGAGCAATGGGCAGCCGGAGAACAACTACAAGACCACG |
| | CCTCCCGTGCTGGACTCCGACGGCTCCTTCTTCCTCTACAGCAAGCTCACCGTGGACA |
| | AGAGCAGGTGGCAGCAGGGGAACGTCTTCTCATGCTCCGTGATGCATGAGGCTCTGCA |
| | CAACCACTACACGCAGAAGAGCCTCTCCCTGTCTCCGGGTAAAGGCGGCGGCGGCGGC |
| | GGCGGCGGCGGGCCAATCTGTGTCAACACCAAGGGGCAAGATGCAAGTACCATTAAAG |
| | ACATGATCACCAGGATGGACCTTGAAAACCTGAAGGATGTGCTCTCCAGGCACAAGCG |
| | GGAGATAGATGTTCTGCAACTGGTGGTGGATGTAGATGGAAACATTGTGAATGAGGTA |
| | AAGCTGCTGAGAAAGGAAAGCCGTAACATGAACTCTCGTGTTACTCAACTCTATATGC |
| | AATTATTACATGAGATTATCCGTAAGAGGGATAATTCACTTGAACTTTCCCAACTGGA |
| | AAACAAAATCCTCAATGTCACCACAGAAATGTTGAAGATGGCAACAAGATACAGGGAA |
| | CTAGAGGTGAAATACGCTTCCTTGACTGATCTTGTCAATAACCAATCTGTGATGATCA |
| | CTTTGTTGGAAGAACAGTGCTTGAGGATATTTTCCCGACAAGACACCCATGTGTCTCC |
| | CCCACTTGTCCAGGTGGTGCCACAACATATTCCTAACAGCCAACAGTATACTCCTGGT |
| | CTGCTGGGAGGTAACGAGATTCAGAGGGATCCAGGTTATCCCAGAGATTTAATGCCAC |
| | CACCTGATCTGGCAACTTCTCCCACCAAAAGCCCTTTCAAGATACCACCGGTAACTTT |
| | CATCAATGAAGGACCATTCAAAGACTGTCAGCAAGCAAAAGAAGCTGGGCATTCGGTC |
| | AGTGGGATTTATATGATTAAACCTGAAAACAGCAATGGACCAATGCAGTTATGGTGTG |
| | AAAACAGTTTGGACCCTGGGGGTTGGACTGTTATTCAGAAAAGAACAGACGGCTCTGT |
| | CAACTTCTTCAGAAATTGGGAAAATTATAAGAAAGGGTTTGGAAACATTGACGGAGAA |
| | TACTGGCTTGGACTGGAAAATATCTATATGCTTAGCAATCAAGATAATTACAAGTTAT |
| | TGATTGAATTAGAAGACTGGAGTGATAAAAAAGTCTATGCAGAATACAGCAGCTTTCC |
| | TCTGGAACCTGAAAGTGAATTCTATAGACTGCGCCTGGGAACTTACCAGGGAAATGCA |
| | GGGGATTCTATGATGTGGCATAATGGTAAACAATTCACCACACTGGACAGAGATAAAG |
| | ATATGTATGCAGGAAACTGCCCCCACTTTCATAAAGGAGGCTGGTGGTACAATGCCTG |
| | TGCACATTCTAACCTAAATGGAGTATGGTACAGAGGAGGCCATTACAGAAGCAAGCAC |
| | CAAGATGGAATTTTCTGGGCCGAATACAGAGGCGGGTCATACTCCTTAAGAGCAGTTC |
| | AGATGATGATCAAGCCTATTGACTGA |

| | |
|---|---|
| ORF Start: at 1 | ORF Stop: TGA at 1996 |

| | | |
|---|---|---|
| SEQ ID NO: 38 | 665 aa | MW at 75585.0 kD |

| | |
|---|---|
| NOV1s, | DKTHTCPPCPAPELLGGPSVFLFPPKPKDTLMISRTPEVTCVVVDVSHEDPEVKFNWY |
| CG57067-18 | |
| Protein | VDGVEVHNAKTKPREEQYNSTYRVVSVLTVLHQDWLNGKEYKCKVSNKALPAPIEKTI |
| Sequence | |
| | SKAKGQPREPQVYTLPPSRDELTKNQVSLTCLVKGFYPSDIAVEWESNGQPENNYKTT |
| | PPVLDSDGSFFLYSKLTVDKSRWQQGNVFSCSVMHEALHNHYTQKSLSLSPGKGGGGG |
| | GGGGPICVNTKGQDASTIKDMITRMDLENLKDVLSRQKREIDVLQLVVDVDGNTVNEV |
| | KLLRKESRNMNSRVTQLYMQLLHEIIRKRDNSLELSQLENKILNVTTEMLKMATRYRE |
| | LEVKYASLTDLVNNQSVMTTLLEEQCLRIFSRQDTHVSPPLVQVVPQHIPNSQQYTPG |
| | LLGGNEIQRDPGYPRDLMPPPDLATSPTKSPFKIPPVTFINEGPFKDCQQAKEAGHSV |
| | SGIYMIKPENSNGPMQLWCENSLDPGGWTVIQKRTDGSVNFFRNWENYKKGFGNIDGE |
| | YWLGLENIYMLSNQDNYKLLIELEDWSDKKVYAEYSSFRLEPESEFYRLRLGTYQGNA |
| | GDSMMWHNGKQFTTLDRDKDMYAGNCAHFHKGGWWYNACAHSNLNGVWYRGGHYRSKH |
| QDGIFWAEYRGGSYSLRAVQMMIKPID | |

| | |
|---|---|
| SEQ ID NO:39 | 1767 bp |

| | |
|---|---|
| NOV1t, | GACAAAACTCACACATGCCCACCGTGCCCAGCACCTGAACTCCTGGGGGGACCGTCAG |
| CG57067-19 | |
| DNA Sequence | TCTTCCTCTTCCCCCCAAAACCCAAGGACACCCTCATGATCTCCCGGACCCCTGAGGT |
| | CACATGCGTGGTGGTGGACGTGAGCCACGAAGACCCTGAGGTCAAGTTCAACTGGTAC |
| | GTGGACGGCGTGGAGGTGCATAATGCCAAGACAAAGCCGCGGGAGGAGCAGTACAACA |
| | GCACGTACCGTGTGGTCAGCGTCCTCACCGTCCTGCACCAGGACTGGCTGAATGGCAA |
| | GGAGTACAAGTGCAAGGTCTCCAACAAAGCCCTCCCAGCCCCCATCGAGAAAACCATC |
| | TCCAAAGCCAAAGGGCAGCCCCGAGAACCACAGGTGTACACCCTGCCCCCATCCCGGG |
| | ATGAGCTGACCAAGAACCAGGTCAGCCTGACCTGCCTGGTCAAAGGCTTCTATCCCAG |
| | CGACATCGCCGTGGAGTGGGAGAGCAATGGCCAGCCGGAGAACAACTACAAGACCACG |
| | CCTCCCGTGCTGGACTCCGACGGCTCCTTCTTCCTCTACAGCAAGCTCACCGTGGACA |
| | AGAGCAGGTGGCAGCAGGGGAACGTCTTCTCATGCTCCGTGATQCATGAGGCTCTGCA |
| | CAACCACTACACGCAGAAGAOCCTCTCCCTGTCTCCCGGTAAAGGCGGCGGCGGCGGC |
| | GGCGGCGGCCATGAGATTATCCGTAAGAGGGATAATTCACTTGAACTTTCCCAACTGG |
| | AAAACAAAATCCTCAATGTCACCACAGAAATGTTGAAGATGGCAACAAGATACAGGGA |
| | ACTAGAGGTGAAATACGCTTCCTTGACTGATCTTGTCAATAACCAATCTGTGATGATC |
| | ACTTTGTTGGAAGAACAGTGCTTGAGGATATTTTCCCGACAAGACACCCATGTGTCTC |
| | CCCCACTTGTCCAGGTGGTGCCACAACATATTCCTAACAGCCAACAGTATACTCCTGQ |
| | TCTGCTGGGAGGTAACGAGATTCAGAGGGATCCAGGTTATCCCAGAGATTTAATGCCA |
| | CCACCTGATCTGGCAACTTCTCCCACCAAAAGCCCTTTCAAGATACCACCGGTAACTT |
| | TCATCAATGAAGGACCATTCAAAGACTGTCAGCAAGCAAAAGAAGCTGGGCATTCGGT |
| | CAGTGGGATTTATATGATTAAACCTGAAAACAGCAATGGACCAATGCAGTTATGGTGT |
| | GAAAACAGTTTGGACCCTGGGGGTTGGACTGTTATTCAGAAAAGAACAGACGGCTCTG |
| | TCAACTTCTTCAGAAATTGQGAAAATTATAAGAAAGGGTTTGGAAACATTGACGGAGA |
| | ATACTGGCTTGGACTGGAAAATATCTATATGCTTAGCAATCAAGATAATTACAAGTTA |
| | TTGATTOAATTAGAAGACTGGAGTGATAAAAAAGTCTATGCAGAATACAGCAGCTTTC |
| | GTCTGGAACCTGAAAGTGAATTCTATAGACTGCGCCTGGGAACTTACCAGGGAAATGC |
| | AGGGGATTCTATGATGTGGCATAATGGTAAACAATTCACCACACTGGACAGAGATAAA |
| | GATATGTATGCAGGAAACTGCGCCCACTTTCATAAAGGAGGCTGGTGGTACAATGCCT |
| | GTGCACATTCTAACCTAAATGGAGTATGGTACAGAGGAGGCCATTACAGAAGCAAGCA |
| | CCAAGATGGAATTTTCTGGGCCGAATACAGAGGCGGGTCATACTCCTTAAGAGCAGTT |
| | CAGATGATGATCAAGCCTATTGACTGA |

| | |
|---|---|
| ORF Start: at 1 | ORF Stop: TGA at 1765 |

| | | |
|---|---|---|
| SEQ ID NO: 40 | 588 aa | MW at 66758.8 kD |

| | |
|---|---|
| NOV1t, | DKTHTCPPCPAPELLGGPSVFLFPPKPKDTLMISRTPEVTCVVVDVSHEDPEVKFNWY |
| CG57067-19 | |
| Protein | VDGVEVHNAKTKPREEQYNSTYRVVSVLTVLHQDWLNGKEYKCKVSNKALPAPIEKTI |
| Sequence | |
| | SKAKGQPREPQVYTLPPSRDELTKNQVSLTCLVKGFYPSDIAVEWESNGQPENNYKTT |
| | PPVLDSDGSFFLYSKLTVDKSRWQQGNVFSCSVMHEALHNHYTQKSLSLSPGKGGGGG |
| | GGGHEIIRKRDNSLELSQLENKILNVTTEMLKMATRYRELEVKYASLTDLVNNQSVMI |
| | TLLEEQCLRIFSRQDTHVSPPLVQVVPQHIPNSQQYTPGLLGGNEIQRDPGYPRDLMP |
| | RPDLATSPTKSPFKIPPVTFINEGPFKDCQQAKEAGHSVSGIYMIKPENSNGPMQLWC |
| | ENSLDPGGWTVIQKRTDGSVNFFRNWENYKKGFGNIDGEYWLGLENIYMLSNQDNYKL |
| | LIELEDWSDKKVYAEYSSFRLEPESEFYRLRLGTYQGNAGDSMMWHNGKQFTTLDRDK |
| | DMYAGNCAHFHKGGWWYNACAHSNLNGVWYRGGHYRSKHQDGIFWAEYRGGSYSLRAV |
| | QMMIKPID |

| | |
|---|---|
| SEQ ID NO:41 | 1767 bp |

| | |
|---|---|
| NOV1t, | GACAAAACTCACACATGCCCACCGTGCCCAGCACCTGAACTCCTGGGGGGACCGTCAGTC |
| CG57067-19a | |
| DNA Sequence | TTCCTCTTCCCCCCAAAACCCAAGGACACCCTCATGATCTCCCGGACCCCTGAGGTCACA |
| | TGCGTGGTGGTGGACGTGAGCCACGAAGACCCTGAGGTCAAGTTCCTGGTACGTGGAC |
| | GGCGTGGAGGTGCATAATGCCAAGACAAAGCCGCGGGAGGAGCAGTACAACAGCACGTAC |
| | CGTGTGGTCAGCGTCCTCACCGTCCTGCACCAGGACTGGCTGAATGGCAAGGAGTACAAG |
| | TGCAAGGTCTCCAACAAAGCCCTCCCAGCCCCCATCGAGAAAACCATCTCCAAAGCCAAA |
| | GGGCAGCCCCGAGAACCACAGGTGTACACCCTGCCCCCATCCCGGGATGAGCTGACCAAG |
| | GACCAGGTCAGCCTGACCTGCCTGGTCAAAGGCTTCTATCCCAGCGACATCGCCGTGGAG |
| | TGGGAGAGCAATGGGCAGCCGGAGAACAACTACAAGACCACGCCTCCCGTGCTGGACTCC |
| | GACGGCTCCTTCTTCCTCTACAGCAAGCTCACCGTGGACAAGAGCAGGTGGCAGCAGGGG |
| | AACGTCTTCTCATGCTCCGTGATGCATGAGGCTCTGCACAACCACTACACGCAGACGAGC |
| | CTCTCCCTGTCTCCGGGTAAAGGCGGCGGCGGCGGCGGCGGATCTCATGAGATTATCCGT |
| | AAGAGGGATAATTCACTTGAACTTTCCCAACTGGAAAACAAAATCCTCAATGTCACCACA |
| | GAAATGTTGAAGATGGCAACAAGATACAGGGAACTAGAGGTGAAATACGCTTCCTTGACT |
| | GATCTTGTCAATAACCAATCTGTGATGATCACTTTGTTGGAAGAACAGTGCTTGAGGATA |
| | TTTTCCCGACAAGACACCCATGTGTCTCCCCCACTTGTCCAGGTGGTGCCACAACATATT |
| | CCTAACAGCCAACAGTATACTCCTGGTCTGCTGGGACGTAACGAGATTCAGAGGGATCCA |
| | GGTTATCCCAGAGATTTAATGCCACCACCTGATCTGGCAACTTCTCCCACCAAAAGCCCT |
| | TTCAAGATACCACCGGTAACTTTCATCAATGAAGGACCATTCAAAGACTGTCAGCAAGCA |
| | AAAGAAGCTGGGCATTCGGTCAGTGGGATTTATATGATTAAACCTGAAAACAGCAATGGA |
| | CCAATGCAGTTATGGTGTGAAAAACAGTTTGGACCCTGGGGGTTGGACTGTTATTCAGAA |
| | AGAACAGACGGCTCTGTCAACTTCTTCAGAAATTGGGAAAATTATAAGAAAGGGTTTGGA |
| | AACATTGACGGAGAATACTGGCTTGGACTGGAAAATATCTATATGCTTAGCAATCAAGAT |
| | AATTACAAGTTATTGATTGAATTAGAAGACTGGAGTGATAAAAAAGTCTATGCAGAATAC |
| | AGCAGCTTTCGTCTGGACCTGAAAGTGAATTCTATAGACTGCGCCTGGGAAACTTACCAG |
| | GGAAATGCAGGGGATTCTATGATGTGGCATAATGGTAAACAATTCACCACACTGGACAGA |
| | GATAAAGATATGTATGCAGGAAACTGCGCCCACTTTCATAAAGGAGGCTGGTGGTACAAT |
| | GCCTGTGCACATTCTAACCTAATGGAGTATGGTACAGAGGAGGCCATTACAGAAGCAAAG |
| | CACCAAGATGGAATTTTCTGGGCCGAATACAGAGGCGGGTCATACTCCTTAAGAGCAGTT |
| | CAGATGATGATCAAGCCTATTGACTGA |

| | |
|---|---|
| ORF Start: at 1 | ORF Stop: TGA at 1765 |

| | | |
|---|---|---|
| SEQ ID NO: 42 | 588 aa | MW at 66758.8 kD |

| | |
|---|---|
| NOV1t, | DKTHTCPPCPAPELLGGPSVFLFPPKPKDTLMISTRTPEVTCVVVDVSHEDPEVKFNWY |
| CG57067-19a | |
| Protein | VDGVEVHNAKTKPREEQYNSTYRVVSVLTVLHQDWLNGKEYKCKVSNKALPAPIEKTI |
| Sequence | |
| | SKAKGQPREPWVYTLPPSRDELTKDQVSLTCLVKGFYPSDIAVEWESNGQPENNYKTT |
| | PPVLDSDGSFFLYSKLTVDKSRWQQGNVFSCSVMHEALHNHYTQKSLSLSPGKGGGGG |
| | GGSHEIIRKRDNSLELSQLENKILNVTTEMLKMATRYRELEVKYASLTDLVNNQSVMI |
| | TLLEEQCLRIFSRQDTHVSPPLVQVVPQHIPNSQQYTPGLLGGNEIQRDPGYPRDLMP |
| | PPDLATSPTKSPFKIPPVTFINEGPFKDCQQAKEAGHSVSGIYMIKPENSNGPMQLWC |
| | ENSLDPGGWTVIQKRTDGSVNFFRNWENYKKGFGNIDGEYWLGLENIYMLSNQDNYKL |
| | DMYAGNCAHFHKGGWWYNACAHSNLNGVWYRGGHYRSKHQDGIFWAEYRGGSYSLRAV |
| | QMMIKPID |

| | |
|---|---|
| SEQ ID NO: 43 | 1470 bp |

| | |
|---|---|
| NOV1u, | GACAAAACTCACACATGCCCACCGTGCCCAGCACCTGAACTCCTGGGGGGACCGTCAG |
| CG57067-20 | |
| DNA Sequence | TCTTCCTCTTCCCCCCAAAACCCAAGGACACCCTCATGATCTCCCGGACCCCTGAGGT |
| | CACATGCGTGGTGGTGGACGTGAGCCACGAAGACCCTGAGGTCAAGTTCAACTGGTAC |
| | GTGGACGGCGTGGAGGTGCATAATGCCAAGACAAAGCCGCGGGAGGAGCAGTACAACA |
| | GCACGTACCGTGTGGTCAGCGTCCTCACCGTCCTGCACCAGGACTGGCTGAATGGCAA |
| | GGAGTACAAGTGCAAGGTCTCCAACAAAGCCCTCCCAGCCCCCATCGAGAAAACCATC |
| | TCCAAAGCCAAAGGGCAGCCCCGAGAACCACAGGTGTACACCCTGCCCCCATCCCGGG |
| | ATGAGCTGACCAAGAACCAGGTCAGCCTGACCTGCCTGGTCAAAGGCTTCTATCCCAG |
| | CGACATCGCCGTGGAGTGGGAGAGCAATGGGCAGCCGGAGAACAACTACAAGACCACG |
| | CCTCCCGTGCTGGACTCCGACGGCTCCTTCTTCCTCTACAGCAAGCTCACCGTGGACA |
| | AGAGCAGGTGGCAGCAGGGQAACGTCTTCTCATGCTCCGTGATGCATGAGGCTCTGCA |
| | CAACCACTACACGCAGAAGAGCCTCTCCCTGTCTCCGGGTAAAGGCGGCGGCGGCGGC |
| | GGCGGCGOCGAGATTCAGAGGGATCCAGGTTATCCCAGAGATTTAATGCCACCACCTG |
| | ATCTGGCAACTTCTCCCACCAAAAGCCCTTTCAAGATACCACCGGTAACTTTCATCAA |
| | TGAAGGACCATTCAAAGACTGTCAGCAAGCAAAAGAAGCTGGGCATTCGGTCAGTGGG |
| | ATTTATATGATTAAACCTGAAAACAGCAATGGACCAATGCAGTTATGGTGTGAAAACA |
| | GTTTGGACCCTGGGGGTTGGACTGTTATTCAGAAAAGAACAGACGGCTCTGTCAACTT |
| | CTTCAGAAATTGGGAAAATTATAAGAAAGGGTTTGGAAACATTGACGGAGAATACTGG |
| | CTTGGACTGGAAAATATCTATATGCTTAGCAATCAAGATAATTACAAGTTATTGATTG |
| | AATTACAAGACTGGAGTGATAAAAAAGTCTATGCAGAATACAGCAGCTTTCGTCTGGA |
| | ACCTGAAAGTGAATTCTATAGACTGCGCCTGGGAACTTACCAGGGAAATGCAGGGGAT |
| | TCTATGATGTGGCATAATGGTAAACAATTCACCACACTGGACAGAGATAAAGATATGT |
| | ATGCAGGAAACTGCGCCCACTTTCATAAAGGAGGCTGGTGGTACAATGCCTGTGCACA |
| | TTCTAACCTAAATGGAGTATGGTACAGAGGAGGCCATTACAGAAGCAAGCACCAAGAT |
| | GGAATTTTCTGGGCCGAATACAGAGGCGGGTCATACTCCTTAAGAGCAGTTCAGATGA |
| | TGATCAAGCCTATTGACTGA |

| | |
|---|---|
| ORF Start: at 1 | ORF Stop: TGA at 1468 |

| | | |
|---|---|---|
| SEQ ID NO: 44 | 489 aa | MW at 55389.9 kD |

| | |
|---|---|
| NOV1u, | DKTHTCPPCPAPELLGGPSVFLFPPKPKDTLMISRTPEVTCVVVDVSHEDPEVKFNWY |
| CG57067-20 | |
| Protein | VDGVEVHNAKTKPREEQYNSTYRVVSVLTVLHQDWLNGKEYKCKVSNKALPAPIEKTI |
| Sequence | |
| | SKAKGQPREPQVYTLPPSRDELTKNQVSLTCLVKGFYPSDIAVEWESNGQPENNYKTT |
| | PPVLDSDGSFFLYSKLTVDKSRWQQGNVFSCSVMHEALHNHYTQKSLSLSPGKGGGGG |
| | GGGEIQRDPGYPRDLMPPPDLATSPTKSPFKIPPVTFINEGPFKDCQQAKEAGHSVSG |
| | IYMIKPENSNGPMQLWCENSLDPGGWTVIQKRTDGSVNFFRNWENYKKGFGNIDGEYW |
| | LGLENIYMLSNQDNYKLLTELEDWSDKKVYAEYSSFRLEPESEFYRLRLGTYQGNAGD |
| | SMMWHNGKQFTTLDRDKDMYAGNCAHFHKGGWWYNACAHSNLNGVWYRCGHYRSKHQD |
| | GIFWAEYRGGSYSLRAVQMMIKPID |

| | |
|---|---|
| SEQ ID NO:45 | 1389 bp |

| | |
|---|---|
| NOV1v, | GACAAAACTCACACATGCCCACCGTGCCCAGCACCTGAACTCCTGGGGGGACCGTCAG |
| CG57067-21 | |
| DNA Sequence | TCTTCCTCTTCCCCCCAAAACCCAAGGACACCCTCATGATCTCCCGGACCCCTGAGGT |
| | CACATGCGTGGTGGTGGACGTGAGCCACGAAGACCCTGAGGTCAAGTTCAACTGGTAC |
| | GTGGACGGCGTGGAGGTGCATAATGCCAAGACAAAGCCGCGGGAGGAGCAGTACAACA |
| | GCACGTACCGTGTGGTCAGCGTCCTCACCGTCCTGCACCAGGACTGGCTGAATGGCAA |
| | GGAGTACAAGTGCAAGGTCTCCAACAAAGCCCTCCCAGCCCCCATCGAGAAAACCATC |
| | TCCAAAGCCAAAGGGCAGCCCCGAGAACCACAGGTGTACACCCTGCCCCCATCCCGGG |
| | ATGAGCTGACCAAGAACCAGGTCAGCCTGACCTGCCTGGTCAAAGGCTTCTATCCCAG |
| | CGACATCGCCGTGGAGTGGGAGAGCAATGGGCAGCCGGAGAACAACTACAAGACCACG |
| | CCTCCCGTOCTGGACTCCGACGGCTCCTTCTTCCTCTACAGCAAGCTCACCGTGGACA |
| | AGAGCAGGTGGCAGCAGGGGAACGTCTTCTCATGCTCCGTGATGCATGAGGCTCTGCA |
| | CAACCACTACACGCAGAAGAGCCTCTCCCTGTCTCCGGGTAAAGGCGGCGGCGGCGGC |
| | GGCGGCGCCAAGATACCACCGGTAACTTTCATCAATGAAGGACCATTCAAAGACTGTC |
| | AQCAAGCAAAAGAAGCTGGGCATTCGGTCAGTGGGATTTATATGATTAAACCTGAAAA |
| | CAGCAATGGACCAATGCAGTTATGGTGTGAAAACAGTTTGGACCCTGGGGGTTGGACT |
| | GTTATTCAGAAAAGAACAGACGGCTCTGTCAACTTCTTCAGAAATTGGGAAAATTATA |
| | AGAAAGGGTTTGGAAACATTGACGGAGAATACTGGCTTGGACTGGAAAATATCTATAT |
| | GCTTAGCAATCAAGATAATTACAAGTTATTGATTGAATTAGAAGACTGGAGTGATAAA |
| | AAAGTCTATGCAGAATACAGCAGCTTTCGTCTGGAACCTGAAAGTGAATTCTATAGAC |
| | TGCGCCTGGGAACTTACCAGGGAAATGCAGGGGATTCTATGATGTGGCATAATGGTAA |
| | ACAATTCACCACACTGGACAGAGATAAAGATATGTATGCAGGAAACTGCGCCCACTTT |
| | CATAAAGGAGGCTGGTGGTACAATGCCTGTGCACATTCTAACCTAAATGGAGTATGGT |
| | ACAGAGGAGGCCATTACAGAAGCAAGCACCAAGATGGAATTTTCTGGGCCGAATACAG |
| | AGGCGGGTCATACTCCTTAAGAGCAGTTCAGATGATGATCAAGCCTATTGACTGA |

| | |
|---|---|
| ORF Start: at 1 | ORF Stop: TGA at 1387 |

| | | |
|---|---|---|
| SEQ ID No:46 | 462 aa | MW at 52381.5 kD |

| | |
|---|---|
| NOV1v, | DKTHTCPPCPAPELLGGPSVFLFPPKPKDTLMISRTPEVTCVVVDVSHEDPEVKFNWY |
| CG57067-21 | |
| Protein | VDGVEVHNAKTKPREEQYNSTYRVVSVLTVLHQDWLNGKEYKCKVSNKALPAPIEKTI |
| Sequence | |
| | SKAKGQPREPQVYTLPPSRDELTKNQVSLTCLVKGFYPSDIAVEWESNGQPENNYKTT |
| | PPVLDSDGSFFLYSKLTVDKSRWQQGNVFSCSVMHEALHNHYTQKSLSLSPGKGGGGG |
| | GGGKIPPVTFINEGPFKDCQQAKEAGHSVSGIYMIKPENSNGPMQLWCENSLDPGGWT |
| | VIQKRTDGSVNFFRNWENYKKGFGNIDGEYWLGLENIYMLSNQDNYKLLIELEDWSDK |
| | KVYAEYSSFRLEPESEFYRLRLGTYQGNAGDSMMWHNGKQFTTLDRDKDMYAGNCAHF |
| | HKGGWWYNACAHSNLWGVWYRGGHYRSKHQDGIFWAEYRGGSYSLRAVQMMIKPID |

| | |
|---|---|
| SEQ ID NO:47 | 2094 bp |

| | |
|---|---|
| NOV1w, | GACAAAACTCACACATGCCCACCGTGCCCAGCACCTGAACTCCTGGGGGGACCGTCAG |
| CG57067-22 | |
| DNA Sequence | TCTTCCTCTTCCCCCCAAAACCCAAGGACACCCTCATGATCTCCCGGACCCCTGAGGT |
| | CACATGCGTGGTGGTGGACGTGAGCCACGAAGACCCTGAGGTCAAGTTCAACTGGTAC |
| | GTGGACGGCGTGCAGGTGCATAATGCCAAGACAAAGCCGCGGGAGGAGCAGTACAACA |
| | GCACGTACCGTGTGGTCAGCGTCCTCACCGTCCTGCACCAGGACTGGCTGAATGGCAA |
| | GGAGTACAAGTGCAAGGTCTCCAACAAAGCCCTCCCAGCCCCCATCGAGAAAACCATC |
| | TCCAAAGCCAAAGGGCAGCCCCGAGAACCACAGGTGTACACCCTGCCCCCATCCCGGG |
| | ATGAGCTGACCAAGAACCAGGTCAGCCTGACCTGCCTGGTCAAAGGCTTCTATCCCAG |
| | CGACATCGCCGTGGAGTGGGAGAGCAATGGGCAGCCGGAGAACAACTACAAGACCACG |
| | CCTCCCGTGCTGGACTCCGACGGCTCCTTCTTCCTCTACAGCAAGCTCACCGTGGACA |
| | AGAGCAGGTGGCAGCAGGGGAACGTCTTCTCATQCTCCGTGATGCATGAGGCTCTGCA |
| | CAACCACTACACGCAGAAGAGCCTCTCCCTGTCTCCGGGTAAAGGCGGCGGCGGCGCC |
| | GGCGGCGGCAAGATACCACCGGTAACTTTCATCAATGAAGGACCATTCAAAGACTGTC |
| | AGCAAGCAAAAGAAGCTGGGCATTCGGTCAGTGGGATTTATATGATTAAACCTGAAAA |
| | CAGCAATGGACCAATGCAGTTATGGTGTGAAAACAGTTTGGACCCTGGGGGTTGGACT |
| | GTTATTCAGAAAAGAACAGACGGCTCTGTCAACTTCTTCAGAAATTGGGAAAATTATA |
| | AGAAAGGGTTTGGAAACATTGACGGAGAATACTGGCTTGGACTGGAAAATATCTATAT |
| | GCTTAGCAATCAAGATAATTACAAGTTATTGATTGAATTAGAAGACTGGAGTGATAAA |
| | AAAGTCTATGCAGAATACAGCAGCTTTCGTCTGGAACCTGAAAGTGAATTCTATAGAC |
| | TGCGCCTGGGAACTTACCAGGGAAATGCAGGGGATTCTATGATGTGGCATAATGGTAA |
| | ACAATTCACCACACTGGACAGAGATAAAGATATGTATGCAGGAAACTGCGCCCACTTT |
| | CATAAAGGAGGCTGGTGGTACAATGCCTGTGCACATTCTAACCTAAATGGAGTATCGT |
| | ACAGAGGAGGCCATTACAGAAGCAAGCACCAAGATGGAATTTTCTGGGCCGAATACAG |
| | AGGCGGGTCATACTCCTTAAGAGCAGTTCAGATGATGATCAAGCCTATTGACGGCGGC |
| | GGCGGCGGCGGCGGCGGCGACAAAACTCACACATGCCCACCGTGCCCAGCACCTGAAC |
| | TCCTGGGGGGACCGTCAGTCTTCCTCTTCCCCCCAAAACCCAAGGACACCCTCATGAT |
| | CTCCCGGACCCCTGAGGTCACATGCGTGGTGGTGGACGTGAGCCACGAAGACCCTGAG |
| | GTCAAGTTCAACTGGTACGTGGACGGCGTGGAGGTGCATAATGCCAAGACAAAGCCGC |
| | GGGAGGAGCAGTACAACAGCACGTACCGTGTGGTCAGCGTCCTCACCGTCCTGCACCA |
| | GGACTGGCTGAATGGCAAGGAGTACAAGTGCAAGGTCTCCAACAAAGCCCTCCCAGCC |
| | CCCATCGAGAAAACCATCTCCAAAGCCAAAGGGCAGCCCCGAGAACCACAGGTGTACA |
| | CCCTGCCCCCATCCCGGGATGAGCTGACCAAGAACCAGGTCAGCCTGACCTGCCTGGT |
| | CAAAGGCTTCTATCCCAGCGACATCGCCGTGGAGTGGGAGAGCAATGGGCAGCCGGAG |
| | AACAACTACAAGACCACGCCTCCCGTGCTGGACTCCGACGGCTCCTTCTTCCTCTACA |
| | GCAAGCTCACCGTGGACAAGAGCAGGTGGCAGCAGGGGAACGTCTTCTCATGCTCCGT |
| | GATGCATGAGGCTCTGCACAACCACTACACGCAGAAGAGCCTCTCCCTGTCTCCGGGT |
| | AAATGA |

| | |
|---|---|
| ORF Start: at 1 | ORF Stop: TGA at 2092 |

| | | |
|---|---|---|
| SEQ ID NO: 48 | 697 aa | MW at 78367.7 kD |

| | |
|---|---|
| NOV1w, | DKTHTCPPCPAPELLGGPSVFLFPPKPKDTLMISRTPEVTCVVVDVSHEDPEVKFNWY |
| CG57067-22 | |
| Protein | VDGVEVHNAKTKPREEQYNSTYRVVSVLTVLHQDWLNGKEYKCKVSNKALPAPIEKTI |
| Sequence | |
| | SKAKGQPREPQVYTLPPSRDELTKNQVSLTCLVKGFYPSDIAVEWESNGQPENNYKTT |
| | PPVLDSDGSFFLYSKLTVDKSRWQQGNVFSCSVMHEALHNHYTQKSLSLSPGKGGGGG |
| | GGGKIPPVTFINEGPFKDCQQAKEAGHSVSGIYMIKPENSNGPMQLWCENSLDPGGWT |
| | VIQKRTDGSVNFFRNWENYKKGFGNIDGEYWLGLENIYMLSNQDNYKLLIELEDWSDK |
| | KVYAEYSSFRLEPESEFYRLRLGTYQGNAGDSMMWHNGKQFTTLDRDKDMYAGNCAHF |
| | HKGGWWYNACAHSNLNGVWYRGGHYRSKHQDGIFWAEYRGGSYSLRAVQMMIKPIDGG |
| | GGGGGGDKTHTCPPCPAPELLGGPSVFLFPPKPKDTLMISRTPEVTCVVVDVSHEDPE |
| | VKFNWYVDGVEVHNAKTKPREEQYNSTYRVVSVLTVLHQDWLNGKEYKCKVSNKALPA |
| | PIEKTISKAKGQPREPQVYTLPPSRDELTKNQVSLTCLVKGFYPSDIAVEWESNGQPE |
| | NNYKTTPPVLDSDGSFFLYSKLTVDKSRWQQGNVFSCSVMHEALHNHYTQKSLSLSPG |
| | K |

| | |
|---|---|
| SEQ ID NO: 49 | 1308 bp |

| | |
|---|---|
| NOV1x, | GACAAAACTCACACATGCCCACCGTGCCCAGCACCTGAACTCCTGGGGGGACCGTCAG |
| CG57067-24 | |
| DNA Sequence | TCTTCCTCTTCCCCCCAAAACCCAAGGACACCCTCATGATCTCCCGGACCCCTGAGGT |
| | CACATGCGTGGTGGTGGACGTGAGCCACGAAGACCCTGAGGTCAAGTTCAACTGGTAC |
| | GTGGACGGCGTGGAGGTGCATAATGCCAAGACAAAGCCGCGGGAGGAGCAGTACAACA |
| | GCACGTACCGTGTGGTCAGCGTCCTCACCGTCCTGCACCAGGACTGGCTGAATGGCAA |
| | GGAGTACAAGTGCAAGGTCTCCAACAAAGCCCTCCCAGCCCCCATCGAGAAAACCATC |
| | TCCAAAGCCAAAGGGCAGCCCCGAGAACCACAGGTGTACACCCTGCCCCCATCCCGGG |
| | ATGAGCTGACCAAGAACCAGGTCAGCCTGACCTGCCTGGTCAAAGGCTTCTATCCCAG |
| | CGACATCGCCGTGGAGTGGGAGAGCAATGGGCAGCCGGAGAACAACTACAAGACCACG |
| | CCTCCCGTGCTGGACTCCGACGGCTCCTTCTTCCTCTACAGCAAGCTCACCGTGGACA |
| | AGAGCAGGTGGCAGCAGOGGAACGTCTTCTCATGCTCCGTGATGCATGAGGCTCTGCA |
| | CAACCACTACACGCAGAAGAGCCTCTCCCTGTCTCCGGQTAAAGGCGGCGGCGGCGGC |
| | GGCGGCGGCATGAAGACTTTTACCTGGACCCTAGGTGTGCTATTCTTCCTACTAGTGG |
| | ACACTGGACATTGCAGAGGTGGACAATTCAAAATTAAAAAAATAAACCAGAGAACATA |
| | CCCTCGTGCCACAGATGGTAAAGAGGAAGCAAAGAAATGTGCATACACATTCCTGGTA |
| | CCATTAAAGACATGATCACCAGGATGGACCTTGAAAACCTGAAGGATGTGCTCTCCAG |
| | GCAGAAGCGGGAGATAGATGTTCTGCAACTGGTGGTGGATGTAGATGGAAACATTGTG |
| | AATGAGGTAAAGCTGCTGAGAAAGGAAAGCCGTAACATGAACTCTCGTGTTACTCAAC |
| | TCTATATGCAATTATTACATGAGATTATCCGTAAGAGGGATAATTCACTTGAACTTTC |
| | CCAACTGGAAAACAAAATCCTCAATGTCACCACAGAAATGTTGAAGATGGCAACAAGA |
| | TACAGGGAACTAGAGGTGAAATACGCTTCCTTGACTGATCTTGTCAATAACCAATCTG |
| | TGATGATCACTTTGTTGGAAGAACAGTGCTTG |

| | |
|---|---|
| ORF Start: at 1 | ORF Stop: end of sequence |

| | | |
|---|---|---|
| SEQ ID NO:50 | 436 aa | MW at 49356.2 kD |

| | |
|---|---|
| NOV1x, | DKTHTCPPCPAPELLGGPSVFLFPPKPKDTLMISRTPEVTCVVVDVSHEDPEVKFNWY |
| CG57067-24 | |
| Protein | VDGVEVHNAKTKPREEQYNSTYRVVSVLTVLHQDWLNGKEYKCKVSNKALPAPIEKTI |
| Sequence | |
| | SKAKGQPREPQVYTLPPSRDELTKNQVSLTCLVKGFYPSDIAVEWESNGQPENNYKTT |
| | PPVLDSDGSFFLYSKLTVDKSRWQQGNVFSCSVMHEALHNHYTQKSLSLSPGKGGGGG |
| | GGGMKTFTWTLGVLFFLLVDTGHCRGGQFKIKKINQRRYPRATDGKEEAKKCAYTFLV |
| | PEQRITGPICVNTKGQDASTIKDMITRMDLENLKDVLSRQKREIDVLQLVVDVDGNIV |
| | NEVKLLRKESRNMNSRVTQLYMQLLHEIIRKRDNSLELSQLENKILNVTTEMLKMATR |
| | YRELEVKYASLTDLVNNQSVMITLLEEQCL |

| | |
|---|---|
| SEQ ID NO: 51 | 1578 bp |

| | |
|---|---|
| NOV1y, | GACAAAACTCACACATGCCCACCGTGCCCAGCACCTGAACTCCTGGGGGGACCGTCAG |
| CG57067-25 | |
| DNA Sequence | TCTTCCTCTTCCCCCCAAAACCCAAGGACACCCTCATGATCTCCCGGACCCCTGAGGT |
| | CACATGCGTGGTGGTGGACGTGAGCCACGAAAGACCCTGAGGTCAAGTTCAACTGGTAC |
| | GTGGACGGCGTGGAGGTGCATAATGCCAAGACAAAGCCGCGGGAGGAGCAGTACAACA |
| | GCACGTACCGTGTGGTCAGCGTCCTCACCGTCCTGCACCAGGACTGGCTGAATGGCAA |
| | GGAGTACAAGTGCAAGGTCTCCAACAAAGCCCTCCCAGCCCCCATCGAGAAAACCATC |
| | TCCAAAGCCAAAGGGCAGCCCCGAGAACCACAGGTGTACACCCTGCCCCCATCCCGGG |
| | ATGAGCTGACCAAGAACCAGGTCAGCCTGACCTGCCTGGTCAAAGGCTTCTATCCCAG |
| | CGACATCGCCGTGGAGTGGGAGAGCAATGGGCAGCCGGAGAACAACTACAAGACCACG |
| | CCTCCCGTGCTGGACTCCGACGGCTCCTTCTTCCTCTACAGCAAGCTCACCGTGGACA |
| | AGAGCAGGTGGCAGCAGGGGAACGTCTTCTCATGCTCCGTGATGCATGAGGCTCTGCA |
| | CAACCACTACACGCAGAAGAGCCTCTCCCTGTCTCCGGGTAAAGGCGGCGGCGGCGGC |
| | GGCGGCGGCAGGATATTTTCCCGACAAGACACCCATGTGTCTCCCCCACTTGTCCAGG |
| | TGGTGCCACAACATATTCCTAACAGCCAACAGTATACTCCTGGTCTGCTGGGAGGTAA |
| | CGAGATTCAGAGGGATCCAGGTTATCCCAGAGATTTAATGCCACCACCTGATCTGGCA |
| | ACTTCTCCCACCAAAGCCCTTTCAAGATACCACCGGTAACTTTCATCAATGAAAGGAC |
| | CATTCAAAGACTGTCAGCAAGCAAAAGAAGCTGGGCATTCGGTCAGTGGGATTTATAT |
| | GATTAAACCTGAAAACAGCAATGGACCAATGCAGTTATGGTGTGAAAACAGTTTGGAC |
| | CCTGGGGGTTGGACTGTTATTCAGAAAGAACAGACGGCTCTGTCAAACTTCTTCAGAA |
| | ATTGGGAAAATTATAAGAAAGGGTTTGGAAACATTGACGGAGAATACTGGCTTGGACT |
| | GGAAAATATCTATATGCTTAGCAATCAAGATAATTACAAGTTATTGATTGAATTAGAA |
| | GACTGGAGTGATAAAAAAGTCTATGCAGAATACAGCAGCTTTCGTCTGGAAACCTGAA |
| | GTGAATTCTATAGACTGCGCCTGGGAACTTACCAGGGAAATGCAGGGGATTCTATGAT |
| | GTGGCATAATGGTAAACAATTCACCACACTGGACAGAGATAAAGATATGTATGCAGGA |
| | AACTGCGCCCACTTTCATAAAGGAGGCTGGTGGTACAATGCCTGTGCACATTCTAACC |
| | TAAATGGAGTATGGTACAGAOGAGGCCATTACAGAAGCAGCAACCAAGATGGAATTTT |
| | CTGGGCCGAATACAGAGGCGGGTCATACTCCTTAAGAGCAGTTCAGATGATGATCAAG |
| | CCTATTGACGGC |

| | |
|---|---|
| ORF Start: at 1 | ORF Stop: end of sequence |

| | | |
|---|---|---|
| SEQ ID NO: 52 | 526 aa | MW at 59410.4 kD |

| | |
|---|---|
| NOV1y, | DKTHTCPPCPAPELLGGPSVFLFPPKPKDTLMISRTPEVTCVVVDVVSHEDPEVKFNWY |
| CG57067-25 | |
| Protein | VDGVEVHNAKTKPREEQYNSTYRVVSVLTVLHQDWLNGKEYKCKVSNKALPAPIEKTI |
| Sequence | |
| | SKAKGQPREPQVYTLPPSRDELTKNQVSLTCLVKGFYPSDIAVEWESNGQPENNYKTT |
| | PPVLDSDGSFFLYSKLTVDKSRWQQGNVFSCSVMHEALHNHYTQKSLSLSPGKGGGGG |
| | GGGRTFSRQDTHVSPPLVQVVPQHIPMSQQYTPGLLGGNETQRDPGYPRDLMPPPDLA |
| | TSPTKSPFKIPPVTFINEGPFKDCQQAKEAGHSVSGIYMIKPENSNGPMQLWCENSLD |
| | PGGWTVIQKRTDGSVNFFRNWENYKKGFGNIDGEYWLGLENIYMLSNQDNYKLLIELE |
| | DWSDKKVYAEYSSFRLEPESEFYRLRLGTYQGNAGDSMMWHNGKQFTTLDRDKDMYAG |
| | NCAHFHKGGWWYNACAHSNLNGVWYRGGHYRSKHQDGIFWAEYRGGSYSLRAVQMMIK |
| | PIDG |

| | |
|---|---|
| SEQ ID NO: 53 | 603bp |

| | |
|---|---|
| NOV1z, | ATGAAGACTTTTACCTGGACCCTAGGTGTGCTATTCTTCCTACTAGTCGACACTGGAC |
| CG57067-26 | |
| DNA Sequence | ATTGCAGAGGTGGACAATTCAAAATTAAAAAAATAAACCAGAGAAGATACCCTCGTGC |
| | CACAGATGGTAAAGAGGAAGCAAAGAAATGTGCATACACATTCCTGGTACCTGAACAA |
| | AGAATAACAGGGCCAATCTGTGTCAACACCAAGGGGCAAGATGCAAGTACCATTAAAG |
| | ACATGATCACCAGGATGGACCTTGAAAACCTGCTGGATGTGCTCTCCAGGCAGAAGCG |
| | GGAGATAGATGTTCTGCACGTGGTGGTGGATGTAGATGGAAACATTGTGAATGAGGTA |
| | AAGCTGCTGAGAAAGGAAAGCCGTAACATGAACTCTCGTGTTACTCAACTCTATATGC |
| | AATTATTACATGAGATTATCCGTAAGAGGGATAATTCACTTGAACTTTCCCAACTGGA |
| | AAACAAAATCCTCAATGTCACCACAGAAATGTTGAAGATGGCAACAAGATACAGGGAA |
| | CTAGAGGTGAAATACGCTTCCTTGACTGATCTTGTCAATAACCAATCTGTGATGATCA |
| | CTTTGTTGGAAGAACAGTGCTTG |

| | |
|---|---|
| ORF Start: ATG at 1 | ORF Stop: end of sequence |

| | | |
|---|---|---|
| SEQ ID NO: 54 | 201 aa | MW at 23370.0 kD |

| | |
|---|---|
| NOV1z, | MKTFTWTLGVLFFLLVDTGHCRGGQFKIKKINQRRYPRATDGKEEAKKCAYTFLVPEQ |
| CG57067-26 | |
| Protein | RITGPICVNTKGQDASTIKDMITRMDLENLKDVLSRQKREIDVLQLVVDVDGNIVNEV |
| Sequence | |
| | KLLRKESRNMNSRVTQLYMQLLHEIIRKRDNSLELSQLENKILHVTTEMLKMATRYRE |
| | LEVKYASLTDLVNNQSVMITLLEEQCL |

| | |
|---|---|
| SEQ ID NO: 55 | 870 bp |

| | |
|---|---|
| NOV1aa, | AGGATATTTTCCCGACAAGACACCCATGTGTCTCCCCCACTTGTCCAGGTGGTGCCAC |
| CG57067-27 | |
| DNA Sequence | AACATATTCCTAACAGCCAACAGTATACTCCTGGTCTGCTGGGAGGTAACGAGATTCA |
| | GAGGGATCCAGGTTATCCCAGAGATTTAATGCCACCACCTGAATCTGGCACTTCTCCC |
| | ACCAAAAGCCCTTTCAAGATACCACCGGTAACTTTCATCAATGAAGGACCATTCAAAG |
| | ACTGTCAGCAAGCAAAAGAAGCTGGGCATTCGGTCAGTGGGATTTATATGATTAAACC |
| | TGAAAACAGCAATGGACCAATGCAGTTATGGTGTGAAAACAGTTTGGACCCTGGGGGT |
| | TGGACTGTTATTCAGAAAAGAACAGACGGCTCTGTCAACTTCTTCAGAAATTGGGAAA |
| | ATTATAAGAAAGGGTTTGGAAACATTGACGGAGAATACTGGCTTGGACTGGAAAATAT |
| | CTATATGCTTAGCAATCAAGATAATTACAAGTTATTGATTGAATTAGAAGACTGGAGT |
| | GATAAAAAAGTCTATGCAGAATACAGCAGCTTTCGTCTGGAACCTGAAAGTGAATTCT |
| | ATAGACTGCGCCTGGGAACTTACCAGGGAAATGCAGGGGATTCTATGATGTGGCATAA |
| | TGGTAAACAATTCACCACACTGGACAGAGATAAAGATATGTATGCAGGAAACTGCGCC |
| | CACTTTCATAAAGGAGGCTGGTGGTACAATGCCTGTGCACATTCTAACCTAAATGGAG |
| | TATGGTACAGAGGAGGCCATTACAGAAGCAAGCACCAAGATGGAATTTTCTGGGCCGA |
| | ATACAGAGGCGGGTCATACTCCTTAAGAGCAGTTCAGATGATGATCAAGCCTATTGAC |

| | |
|---|---|
| ORF Start: at 1 | ORF Stop: end of sequence |

| | | |
|---|---|---|
| SEQ ID NO:56 | 290 aa | MW at 33367.2 kD |

| | |
|---|---|
| NOV1ab, | CTGACAGACTAACAGACTGTTCCTTTCCATGGGTCTTTTCTGCAGTCACCGTCCTTGA |
| CG57067-28 | |
| DNA Sequence | CACGAAGCTCTAGCCACCATGGAGACAGACACACTCCTGCTATGGGTACTGCTGCTCT |
| | GGGTTCCAGGTTCCACTGGTGACGCGGCCCAGCCGGCCAGGCGCGCGCGCCGTACGAA |
| | GCTTGGTACCGAGCTAGGATCTGACAAAACTCACACATGCCCACCGTGCCCAGCACCT |
| | GAACTCCTGGGGGGACCGTCAGTCTTCCTCTTCCCCCCAAAACCCAAGGACACCCTCA |
| | TGATCTCCCGGACCCCTGAGGTCACATGCGTGGTGGTGGACGTGAGCCACGAAGACCC |
| | TGAGGTCAAGTTCAACTGGTACGTGGACGGCGTGGAGGTGCATAATGCCAAGACAAAG |
| | CCGCGGGAGGAGCAGTACAACAGCACGTACCGTGTGGTCAGCGTCCTCACCGTCCTGC |
| | ACCAGGACTGGCTGAATGGCAAGGAGTACAAGTGCAAGGTCTCCAACAAAGCCCTCCC |
| | AGCCCCCATCGAGAAAACCATCTCCAAAGCCAAAGGGCAGCCCCGAGAACCACAGGTG |
| | TACACCCTGCCCCCATCCCGGGATGAGCTGACCAAGGACCAGGTCAGCCTGACCTGCC |
| | TGGTCAAAGGCTTCTATCCCAGCGACATCGCCGTGGAGTGGGAGAGCAATGGGCAGCC |
| | GGAGAACAACTACAAGACCACGCCTCCCGTGCTGGACTCCGACGGCTCCTTCTTCCTC |
| | TACAGCAAGCTCACCGTGGACAAGAGCAGGTGGCAGCAGGGGAACGTCTTCTCATGCT |
| | CCGTGATGCATGAGGCTCTGCACAACCACTACACGCAGAAGAGCCTCTCCCTGTCTCC |
| | GGGTAAAGGCGGCGGCGGCGGCGGCGGATCTCATGAGATTATCCGTAAGAGGGATAAT |
| | TCACTTGAACTTTCCCAACTGGAAAACAAAATCCTCAATGTCACCACAGAAATGTTGA |
| | AGATGGCAACAAGATACAGGGAACTAGAGGTGAAATACGCTTCCTTGACTGATCTTGT |
| | CAATAACCAATCTGTGATGATCACTTTGTTGGAAGAACAGTGCTTGAGGATATTTTCC |
| | CGACAAGACACCCATGTGTCTCCCCCACTTGTCCAGGTGGTGCCACAACATATTCCTA |
| | ACAGCCAACAGTATACTCCTGGTCTGCTGGGAGGTAACGAGATTCAGAGGGATCCAGG |
| | TTATCCCAGAGATTTTAATGCCACCACCTGATCTGGCAACTTCTCCCACCAAAAGCCT |
| | TTCAAGATACCACCGGTAACTTTCATCAATGAAGGACCATTCACCGACTGTCAGCAAG |
| | CAAAAGAAGCTGGGCATTCGGTCAGTGGGATTTATATGATTAAACCTGAAAACAGCAA |
| | TGGACCAATGCAGTTATGGTGTGAAAACAGTTTGGACCCTGGCGGTTGGACTGTTATT |
| | CAGAAAAGAACAGACGGCTCTGTCAACTTCTTCAGAAATTGGGAAAATTATAAGAAAG |
| | GGTTTGGAAACATTGACGGAGAATACTGGCTTGGACTGGACAATATCTATATGCTTAG |
| | CAATCAAGATAATTACAAGTTATTGATTGAATTAGAAGACTGGAGTGATAAAAAAGTA |
| | TATGCAGAATACAGCAGCTTTCGTCTGGAACCTGAAAGTGAATTCTATAGACTGCGCC |
| | TGGGAACTTACCAGGGAAATGCAGGGGATTCTATGATGTGGCATAATGGTAAACAATT |
| | CACCACACTGGACAGAGATAAAGATATGTATGCAGGAAACTGCGCCCACTTTCATAAA |
| | GGAGGCTGGTGGTACAATGCCTGTGCACATTCTAGCCTAAATGGAGTATGGTACAGAG |
| | GAGGCCATTACAGAAGCAAGCACCAAGATGGAATTTTCTGGGCCGAATACAGAGGCGG |
| | GTCATACTCCTTAAGAGCAGTTCAGATGATGATCAAGCCTATTGACTGACTCGAGGGT |
| | AAGCCTATCCCTAACCCTCTCCTCGGTCTCGATTCTACGCGTACCGGTCATCACCACC |
| | ATCACCATTGAGTTTAATTCAT |

| | |
|---|---|
| ORF Start: at 197 | ORF Stop: TGA at 1961 |

| | | |
|---|---|---|
| SEQ ID NO: 58 | 588 aa | MW at 66762.8 kD |

| | |
|---|---|
| NOV1ab, | DKTHTCPPCPAPELLGGPSVFLFPPKPKDTLMISRTPEVTCVVVDVSHEDPEVKFNWY |
| CG57067-28 | |
| Protein | VDGVEVJMALTLTREEQYNSTRVVSVLTVLHQDWLNGKEYKCKVSNKALPAPIEKT |
| Sequence | |
| | SKAKGQPREPQVYTLPPSRDELTKDQVSLTCLVKGFYPSDIAVEWESNGQPENNYKTT |
| | PPVLDSDGSFFLYSKLTVDKSRWQQGNVFSCSVMHEALHNHYTQKSLSLSPGKGGGGG |
| | GGSHEIIRKRDNSLELSQLENKILNVTTEMLKMATRYRELEVKYASLTDLVNNQSVMI |
| | TLLEEQCLRIFSRQDTHVSPPLVQVVPQHIPNSQQYTPGLLGGNEIQRDPGYPRDLMP |
| | PPDLATSPTKSPFKTPPVTFINEGPFKDCQQAKEAGHSVSGIYMIKPENSNGPMQLWC |
| | ENSLDPGGWTVIQKRTDGSVNFFRNWENYKKGFGNIDGEYWLGLENIYMLSNQDNYKL |
| | LIELEDWSDKKVYAEYSSFRLEPESEFYRLRLGTYQGNAGDSMMWHNGKQFTTLDRDK |
| | DMYAGNCAHFHKGGWWYNACAHSSLNGVWYRGGHYRSKHQDGIFWAEYRGGSYSLRAV |
| | QMMTKPID |

| | |
|---|---|
| SEQ ID NO: 59 | 1484 bp |

| | |
|---|---|
| NOV1ac, | GCCCTTCCACCATGAAGACTTTTACCTGGACCCTAGGTGTGCTATTCTTCCTACTAGT |
| CG57067-29 | |
| DNA Sequence | GGACACTGGACATTGCAGAGGTGGACAATTCAAAATTAAAAAAATAAACCAGAGAAGA |
| | TACCCTCGTGCCACAGATGGTAAAGAGGAAGCAAAGAAATGTGCATACACATTCCTGG |
| | TACCTGACCAAAGAATAACAGGGCCAATCTGTGTCAACACCAAGGGGCAACATGCAAG |
| | TACCATTAAAGACATGATCACCAGGATGGACCTTGAAAACCTGAAGGATGTGCTCTCC |
| | AGGCAGAAGCGGGAGATAGATGTTCTGCAACTGGTGGTGGATGTAGATGGAAACATTG |
| | TGAATGAGGTAAAGCTGCTGAGAAAGGAAAGCCGTAACATGAACTCTCGTGTTACTCA |
| | ACTCTATATGCAATTATTACATGAGATTATCCGTAAGAGGGATAATTCACTTGAACTT |
| | TCCCAACTGGAAAACAAAATCCTCAATGTCACCACAGAAATGTTGAAGATGGCAACAA |
| | GATACAGGGAACTAGAGGTGAAATACGCTTCCTTGACTGATCTTGTCAATAACCAATC |
| | TGTGATGATCACTTTGTTGGAAGAACAGTGCTTGAGGATATTTTCCCGACAAGACACC |
| | CATGTGTCTCCCCCACTTGTCCAGGTGGTGCCACAACATATTCCTAACAGCCAACAGT |
| | ATACTCCTGGTCTGCTGGGAGGTAACGAGATTCAGAGGGATCCAGGTTATCCCAGACA |
| | TTTAATGCCACCACCTGATCTGGCAACTTCTCCCACCAAAAGCCCTTTCAAGATACCA |
| | CCGGTAACTTTCATCAATGAAGGACCATTCAAAGACTGTCAGCAAGCAAAAGAAGCTG |
| | GGCATTCGGTCAGTGGGATTTATATGATTAAACCTGAAAACAGCAATGGACCAATGCA |
| | GTTATGGTGTGAAAACAGTTTGGACCCTGGGGGTTGGACTGTTATTCAGAAAAGAACA |
| | GACGGCTCTGTCAACTTCTTCAGAAATTGGGAAAATTATAAGAAAGGGTTTGGAAACA |
| | TTGACGGAGAATACTGGCTTGGACTGGAAAATATCTATATGCTTAGCAATCAAGATAA |
| | TTACAAGTTATTGATTGAATTAGAAGACTGGAGTGATAAAAAAGTCTATGCAGAATAC |
| | AGCAGCTTTCGTCTGGAACCTGAAAGTGAATTCTATAGACTGCGCCTGGGAACTTACC |
| | AGGGAAAATGCAGGGGATTCTATGATGTGGCATAATGGTAAACAATTCACCACACTGA |
| | CAGAGATAAAGATATGTATGCAGGAAACTGCGCCCACTTTCATAGGGGAGGCTGGTGG |
| | TACAATGCCTGTGCACATTCTAGCCTAAATGGAGTATGGTACAGAGGAGGCCATTACA |
| | GAAGCAAGCACCAAGATGGAATTTTCTGGGCCGAATACAGAGGCGGGTCATACAAACC |
| | AAGAGCAGTTCAGATGATGATCAAGCCTATTGAC |

| | |
|---|---|
| ORF Start: ATG at 12 | ORF Stop: end of sequence |

| | | |
|---|---|---|
| SEQ ID NO: 60 | 491 aa | MW at 56678.1 kD |

| | |
|---|---|
| NOV1ac, | MKTFTWTLGVLFFLLVDTGHCRGGQFKIKKINQRRYPRATDGKEEAKKCAYTFLVPDQ |
| CG57067-29 | |
| Protein | RITGPICVNTKGQDASTIKDMITRMDLENLKDVLSRQKREIDVLQLVVDVDGNIVNEV |
| Sequence | |
| | KLLRKESRNMNSRVTQLYMQLLHEIIRKRDNSLELSQLENKILNVTTEMLKMATRYRE |
| | LEVKYASLTDLVNNQSVMITLLEEQCLRIFSRQDTHVSPPLVQVVPQHIPNSQQYTPG |
| | LLGGNEIQRDPGYPRDLMPPDLATSPTKSPFKIPPVTFINEGPFKDCQQAAKEAGHSV |
| | SGIYMIKPENSNGPMQLWCENSLDPGGWTVIQKRTDGSVNFFRNWENYKKGFGNIDGE |
| | YWLGLENIYMLSNQDNYKLLIELEDWSDKKVYAEYSSFRLEPESEFYRLRLGTYQGNA |
| | GDSMMWHNGKQFTTLDRDKDMYAGNCAHFHKGGWWYNACAHSSLNGVWYRGGHYRSKH |
| | QDGIFWAEYRGGSYSLPAVQMMIKPID |

| | |
|---|---|
| SEQ ID NO: 61 | 1498 bp |

| | |
|---|---|
| NOV1ad, | CACCAGATCTCCCACCATGAAGACTTTTACCTGGACCCTAGGTGTGCTATTCTTCCTA |
| CG57067-30 | |
| DNA Sequence | CTAGTGGACACTGGACATTGCAGAGGTGGACAATTCAAAATTAAAAAAATAAACCAGA |
| | GAAGATACCCTCGTGCCACAGATGGTAAAGAGGAAGCAAAGAAATGTGCATACACATT |
| | CCTGGTACCTGACCAAAGAATAACAGGGCCAATCTGTGTCAACACCAAGGGGCAAGAT |
| | GCAAGTACCATTAAAGACATGATCACCAGGATGGACCTTGAAAACCTGAAGGATGTGC |
| | TCTCCAGGCAGAAGCGGGAGATAGATGTTCTGCAACTGGTGGTGGATGTAGATGGAAA |
| | CATTGTGAATGAGGTAAAGCTGCTGAGAAAGGAAAGCCGTAACATGAACTCTCGTGTT |
| | ACTCAACTCTATATGCAATTATTACATGAGATTATCCGTAAGAGGGATAATTCACTTG |
| | AACTTTCCCAACTGGAAAACAAAATCCTCAATGTCACCACAGAAATGTTGAAGATGGC |
| | AACAAGATACAGGGAACTAGAGGTGAAATACGCTTCCTTGACTGATCTTGTCAATAAC |
| | CAATCTGTGATGATCACTTTGTTGGAAGAACAGTGCTTGAGGATATTTTCCCGACAAG |
| | ACACCCATGTGTCTCCCCCACTTGTCCAGGTGGTGCCACAACATATTCCTAACAGCCA |
| | ACAGTATACTCCTGGTCTGCTGGGAGGTAACGAGATTCAGAGGGATCCAGGTTATCCC |
| | AGAGATTTAATGCCACCACCTGATCTGGCAACTTCTCCCACCAAAAGCCCTTTCAAGA |
| | TACCACCGGTAACTTTCATCAATGAAGGACCATTCAAAGACTGTCAGCAAGCAAAAGA |
| | AGCTGGGCATTCGGTCAGTGGGATTTATATGATTAAACCTGAAAACAGCAATGGACCA |
| | ATGCAGTTATGGTGTGAAAACAGTTTGGACCCTGGGGGTTGGACTGTTATTCAGAAAA |
| | GAACAGACGGCTCTGTCAACTTCTTCAGAAATTGGGAAAATTATAAGAAAGGGTTTGG |
| | AAACATTGACGGAGAATACTGGCTTGGACTGGAAAATATCTATATGCTTAGCAATCAA |
| | GATAATTACAAGTTATTGATTGAATTAGAAGACTGGAGTGATAAAAAAGTCTATGCAG |
| | AATACAGCAGCTTTCGTCTGGAACCTGAAAGTGAATTCTATAGACTGCGCCTGGGAAC |
| | TTACCAGGGAAATGCAGGGGATTCTATGATGTGGCATAATGGTAAACAATTCACCACA |
| | CTGGACAGAGATAAAGATATGTATGCAGGAAACTGCOCCCACTTTCATAAAGGAGGCT |
| | GGTGGTACAATGCCTGTGCACATTCTAGCCTAAATGGAGTATGGTACAGAGGAGGCCA |
| | TTACAGAAGCAAGCACCAAGATGGAATTTTCTGGGCCGAATACAGAGGCGGGTCATAC |
| | TCCTTAAGAGCAGTTCAGATGATGATCAAGCCTATTGACGTCGACGGC |

| | |
|---|---|
| ORF Start: ATG at 17 | ORF Stop: at 1490 |

| | | |
|---|---|---|
| SEQ ID NO:62 | 491 aa | MW at 56678.1 kD |

| | |
|---|---|
| NOV1ad, | MKTFTWTLGVLFFLLVDTGHCRGGQFKIKKINQRRYPRATDGKEEAKKCAYTFLVPDQ |
| CG57067-30 | |
| Protein | RITGPICVNTKGQDASTIKDMITRMDLENLKDVLSRQKREIDVLQLVVDVDGNIVNEV |
| Sequence | |
| | KLLRKESRNNNSRVTQLYMQLLHEIIRKRDNSLELSQLENKILNVTTEMLKMATRYRE |
| | LEVKYASLTDLVNNQSVMITLLEEQCLRIFSRQDTHVSPPLVQVVPQHIPNSQQYTPG |
| | LLGGNEIQRDPGYPRDLMPPPDLATSPTKSPFKIPPVTFINEGPFKDCQQAKEAGHSV |
| | SGIYMIKPENSNGPMQLWCENSLDPGGWTVIQKRTDGSVNFFRNWENYKKGFGNIDGE |
| | YWLGLENIYMLSNQDNYKLLIELEDWSDKKVYAEYSSFRLEPESEFYRLRLGTYQGNA |
| | GDSMMWHNGKQFTTLDRDKDMYAGNCAHFHKGGWWYNACAHSSLNGVWYRGGHYRSKH |
| | QDGIFWAEYRGGSYSLRAVQMMIKPID |

| | |
|---|---|
| SEQ ID NO 63 | 1062bp |

| | |
|---|---|
| NOV1ae | CATGAGATTATCCGTAAGAGGGATAATTCACTTGAACTTTCCCAACTGGCAAAA |
| CG57067-31 | |
| DNA | TCCTCAATGTCACCACAGAAATGTTGAAGATGGCAACAAGATACAGGGAACTAGAGGT |
| Sequence | |
| | GAAATACGCTTCCTTGACTGATCTTGTCAATAACCAATCTGTGATGATCACTTTGTTG |
| | GAAGAACAGTGCTTGAGGATATTTTCCCGACAAGACACCCATGTGTCTCCCCCACTTG |
| | TCCAGGTGGTGCCACAACATATTCCTAACAGCCAACAGTATACTCCTGGTCTGCTGGG |
| | AGGTAACGAGATTCAGAGGGATCCAGGTTATCCCAGAGATTTAATGCCACCACCTGAT |
| | CTGGCAACTTCTCCCACCAAAAGCCCTTTCAAGATACCACCGGTAACTTTCATCAATG |
| | AAGGACCATTCAAAGACTGTCAGCAAGCAAAAGAAGCTGGGCATTCGGTCAGTGGGAT |
| | TTATATGATTAAACCTGAAAACAGCAATGGACCAATGCAGTTATGGTGTGAAAACAGT |
| | TTGGACCCTGGGGGTTGGACTGTTATTCAGAAAAGAACAGACGGCTCTGTCAACTTCT |
| | TCAGAAATTGGGAAAATTATAAGAAAGGGTTTGGAAACATTGACGGAGAATACTGGCT |
| | TGGACTGGAAAATATCTATATGCTTAGCAATCAAGATAATTACAAGTTATTGATTGAA |
| | TTAGAAGACTGGAGTGATAAAAAAGTCTATGCAGAATACAGCAGCTTTCGTCTGGAAC |
| | CTGAAAGTGAATTCTATAGACTGCGCCTGGGAACTTACCAGGGAAATGCAGGGGATTC |
| | TATGATGTGGCATAATGGTAAACAATTCACCACACTGGACAGAGATAAAGATATGTAT |
| | GCAGGAAACTGCGCCCACTTTCATAAAGGAGGCTGGTGGTACAATGCCTGTGCACATT |
| | CTAACCTAAATGGAGTATGGTACAGAGGAGGCCATTACAGAAGCAAGCACCAAOATGG |
| | AATTTTCTGGGCCGAATACAGAGGCGGGTCATACTCCTTAAGAGCAGTTCAGATGATG |
| | ATCAAGCCTATTGACTGA |

| | |
|---|---|
| SEQ ID NO 64 | 353 aa |

| | |
|---|---|
| NOV 1ac | HEIIRKRDNSLELSQLENKILNVTTEMLKMATRYRELEVKYASLTDLVNNQSVMITLL |
| CG57067-31 | |
| Protein | EEQCLRIFSRQDTHXTSPPLVQVVPQHIPNSQQYTPGLLGGNEIQRDPGYPRDLMPPPD |
| Sequence | |
| | LATSPTKSPFKIPPVTFINEGPFKDCQQAKEAGHSVSGIYMIKPENSNGPMQLWCENS |
| | LDPGGWTVIQKRTDGSVNFFRNWENYKKGFGNIDGEYWLGLENIYMLSNQDNYKLLIE |
| | LEDWSDKKVYAEYSSFRLEPESEFYRLRLGTYQGNAGDSMMWHNGKQFTTLDRDKDMY |
| | AGNCAHFHKGGWWYNACAHSNLNGVWYRGGHYRSKHQDGIFWAEYRGGSYSLRAVQMM |
| | IKPID |

| | |
|---|---|
| SEQ ID NO: 65 | 63 bp |

| | |
|---|---|
| Igk DNA | ATGGAGACAGACACACTCCTGCTATGGGTACTGCTGCTCTGGGTTCCAGGTTCCACTG |
| Sequence | |
| | GTGAC |

| | |
|---|---|
| SEQ ID NO: 66 | 21 aa |

| | |
|---|---|
| Igk Protein | METDTLLLWVLLLWVPGSTGD |
| Sequence | |

| | |
|---|---|
| SEQ ID NO: 67 | 360 bp |

| | |
|---|---|
| Coiled Coil | ACCAGGATGGACCTTGAAAACCTGAAGGATGTGCTCTCCAGGCAGAAGCGGGAGATAG |
| (CC) Domain | |
| DNA | ATGTTCTGCAACTGGTGGTGGATGTAGATGGAPACATTGTGAATGAGGTAAAGCTGCT |
| Sequence | |
| | GAGAAAGGAAAGCCGTAACATGAACTCTCGTGTTACTCAACTCTATATGCAATTATTA |
| | CATGAGATTATCCGTAAGAGGGATAATTCACTTGAACTTTCCCAACTGGAAAACAAAA |
| | TCCTCAATGTCACCACAGAAATGTTGAAGATGOCAACAAGATACAGGGAACTAGAGGT |
| | GAAATACGCTTCCTTGACTGATCTTGTCAATAACCAATCTGTGATGATCACTTTGTTG |
| | GAAGAACAGTGC |

| | |
|---|---|
| SEQ ID NO: 68 | 121 aa |

| | |
|---|---|
| Coiled Coil | ITRMDLENLKDVLSRQKREIDVLQLVVDVDGNIVNEVKLLRKESRNMNSRVTQLMQ |
| (CC) Domain | |
| Protein | LLHEIIRKRDNSLELSQLENKILNVTTEMLKMATRYRELEVKYASLTDLVNNQSVMI |
| Sequence | |
| | TLLEEQC |

| | |
|---|---|
| SEQ ID NO: 69 | 84 bp |

| | |
|---|---|
| Coiled Coil 1 | ATCACCAGGATGGACCTTGAAAACCTGAAGGATGTGCTCTCCAGGCAGAAGCGGGAGA |
| (CCI) domain | |
| DNA | TAGATGTTCTGCAACTGGTGGTGGAT |
| Sequence | |

| | |
|---|---|
| SEQ ID NO: 70 | 29 aa |

| | |
|---|---|
| Coiled Coil 1 | ITRMDLENLKDVLSRQKREIDVLQLVVDV |
| (CC1) domain | |
| Protein | |
| Sequence | |

| | |
|---|---|
| SEQ ID NO:71 | 162bp |

| | |
|---|---|
| Coiled Coil 2 | AATTCACTTGAACTTTCCCAACTGGAAAACAAAATCCTCAATGTCACCACAGAAATGT |
| (CC2) domain | |
| DNA | TGAAGATGGCAACAAGATACAGGGAACTAGAGGTGAAATACGCTTCCTTGACTGATCT |
| Sequence | |
| | TGTCAATAACCAATCTGTGATGATCACTTTGTTGGAAGAACAGTGC |

| | |
|---|---|
| SEQ ID NO: 72 | 53aa |

| | |
|---|---|
| Coiled Coil 2 | SLELSQLENKILNVTTEMLKMATRYRELEVKYASLTDLVNNQSVMITLLEEQC |
| (CC2) domain | |
| Protein | |
| Sequence | |

| | |
|---|---|
| SEQ ID NO: 73 | 867 bp |

| | |
|---|---|
| Fibrinogen | CCTTTCAAGATACCACCGGTAACTTTCATCAATGAAGGACCATTCAAAGACTGTCAGC |
| Binding | |
| Domain (FBD) | AAGCAAAAGAAGCTGC3GCATTCGGTCAGTGGGATTTATATGATTAAACCTGAAAACAG |
| DNA | |
| Sequence | CAATGGACCAATGCAGTTATGGTGTGAAAACAGTTTGGACCCTGGGGGTTGGACTGTT |
| | ATTCAGAAAAGAACAGACGGCTCTGTCAACTTCTTCAGAAATTGGGAAAATTATAAGA |
| | AAGGGTTTGGAAACATTGACGGAGAATACTGGCTTGGACTGGAAAATATCTATATGCT |
| | TAGCAATCAAGATAATTACAAGTTATTGATTGAATTAGAAGACTGGAGTGATAAAAAA |
| | GTCTATGCAGAATACAQCAGCTTTCGTCTGGAACCTGAAAGTGAATTCTATAGACTGC |
| | GCCTGGGAACTTACCAGGGAAATGCAGGGGATTCTATGATGTGGCATAATGGTAAACA |
| | ATTCACCACACTGGACAGAGATAAAGATATGTATGCAGGAAACTGCGCCCACTTTCAT |
| | AAAGGAGGCTGGTGGTACAATGCCTGTGCACATTCTAGCCTAAATGGAGTATGGTACA |
| | GAGGAGGCCATTACAGAAGCAAGCACCAAGATGGAATTTTCTGGGCCGAATACAGAGG |
| | CGGGTCATACTCCTTAAGAGCAGTTCAGATGATGATCAAGCCTATTGAC |

| | |
|---|---|
| SEQ ID NO: 74 | 228aa |

| | |
|---|---|
| Fibrinogen | FKIPPVTFINEGPFKDCQQAKEAGHSVSGIYMIKPENSNGPMQLWCENSLDPG |
| Binding | |
| Domain (FBD) | GWTVIQKRTDGSVNFFRNWENYKKGFGNIDGEYWLGLENIYMLSNQDNYKLLI |
| Protein | |
| Sequence | ELEDWSDKKVYAEYSSFRLEPESEFYRLRLGTYQGNAGDSMMWHNGKQFTTLD |
| | RDKDMYAGNCAHFHKGGWWYNACAHSS/NLNGVWYRGGHYRSKHQDGIFWAEY |
| | RGGSYSLRAVQMMIKPID |

### Example 2

Cloning and Expression of Angioarrestins

Angioarrestin FBD/FC (CG57067-08) Fusion Construct:

Based on the predicted reading frame encoding fibrinogen binding domain (FBD), oligonucleotide primers were designed to amplify FBD region by PCR. The primers used were as follows: forward primer, 5′-AAA TTC GGA TCC GAT CTG GCA ACT TCT CCC-3′ (SEQ ID NO:76); reverse primer, 5′-AAA TTC CTC GAG TGA CCC GCC TCT GTA TTC GGC-3′ (SEQ ID NO:77). The PCR mix contained 100 ng of AngX coding regopm, 75 pmole of primers, 5 μmole of dNTPs, 1 U of Fidelity expand polymerase and 5 μl of Fidelity expand buffer (Boehringer Mannheim, Indianapolis, Ind.). A touchdown PCR was used as per standard protocol. A single PCR product was obtained and cloned into pCEP4/Sec vector as Bam HI and Xho I (Invitrogen, San Diego, Calif.). A Xho I and Sal I containing Fc hinge region was amplified and cloned from fetal brain cDNA library. The amplified Fc portion of human IgG1 spanning the hinge region was cloned downstream of AngXFBD region and the resulting vector was named as pCEP4/Sec/AngXFBD-Fc. This vector contains an in-frame V5 and His6 tag at the 3′-terminus of the coding region.

### Example 3

Expression, Purification and Biochemical Characterization of Recombinant Angioarrestins

The pCEP4/Sec/Angioarrestin vector was transfected into HEK293T cells using Lipofectamine Plus reagent according to manufacturer's instructions (Life Technologies Inc., Rockville, Md.). The cell pellet and supernatant were harvested 72 h after transfection and examined for protein expression by Western blot analysis with an anti-V5 mAb. After initial confirmation of expression, large-scale transfections were carried out in 150-cm² petri dishes using Lipofectamine Plus reagent. The conditioned medium was collected from transfected cells after 72 h, pooled and loaded onto a Protein G affinity column according to manufacturer's instructions (Qiagen, Valencia, Calif.). The bound protein was purified as per manufacturer's instructions. The purified protein from Protein G column migrated as single band ˜70 kDa. In addition to monomeric protein higher molecular weight complex (˜210 kDa) was also observed. The higher molecular weight complex when reduced with 5 mM DTT migrated as a single band corresponding to monomeric ˜70 kDa protein suggesting disulfide linkage. The purified protein was immunoreactive to anti-human Fc antibody. The antibody was reactive to monomeric (˜70 kDa as well as higher molecular weight complex).

Following transient and stable transfections in both CHO-K1 and HEK293-EBNA cells, CG57067-28 proteins and molecular weight standards in 2×SDS-sample buffer were resolved by 4-20% SDS-PAGE gel electrophoresis under standard conditions. Proteins were then blotted to nitrocellulose membranes and detected with an anti-human Fc-HRP antibody using the ECL kit (Amersham, Piscataway, N.J.). CG57067-28 protein was expressed at relatively high levels in the cells, and under non-reducing conditions the protein is a monomer at approximately 70 kD and dimer at approximately 150 kD.

### Example 4

Endothelial Cell BrdU Incorporation Assay

The effect of CG57067-08 on proliferation was assessed using HUVEC. The cells were plated in 96-well flat bottom plates pre-coated with Attachment Factor (Cascade Biologics, Portland, Oreg.) at 3×10⁴ cells/well in 100 μl of Medium 200 (Cascade Biologics, Portland, Oreg.) containing 0.5% FBS. After 24 h of starvation at 37° C., the cells were washed 2× with serum-free medium, and then fed with fresh medium containing 1% FBS with VEGFI₁₆₅ and bFGF (10 ng/ml) (R & D Systems, Minneapolis, Minn.) with and without angioinhibin protein. The cells were pulsed with BrdU for 4 h before harvest. The BrdU assay was performed according to the manufacturer's specification (Roche Molecular Biochemicals, Indianapolis, Ind.).

Results:

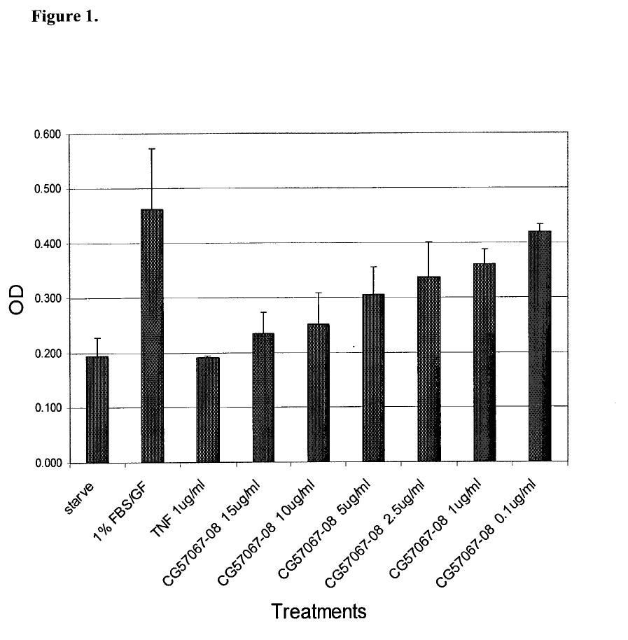

CG57067-08 inhibited VEGF/bFGF-induced BrdU incorporationin HUVEC cells in a dose-dependent manner. (FIG. 1). Maximum inhibition was obtained at 5 μg/ml corresponding to >80% inhibition over control.

### Example 5

Endothelial Cell Adhesion Assay

Untreated 96-well flat bottom tissue culture plates (Fisher Scientific, Springfield, N.J.) were used in the cell adhesion assay. The plates were coated with 10 μg/ml of different extracellular matrix (ECM) proteins (Type I collagen, Type IV collagen, fibronectin, vitronectin, laminin and Matrigel) overnight at 4° C. The remaining protein binding sites were blocked with 1% BSA in PBS, pH 7.4 for 2 h at 37° C. HUVEC cells were grown to subconfluence (70-80%) in Medium 200. The cells were labeled with Calcein-AM fluorophore (Molecular Probes, Eugene, Oreg.). The cells were trypsinized, washed and resuspended at 1.5×10⁵ cells/ml in serum-free medium containing 1% BSA. The cells were then mixed with different concentrations of angioarrestin in 100-μl volumes containing 2×10⁴ cells/treatment for 15 min at room temperature. After incubation, the cell suspension was then added to each well and the plates were incubated at 37° C. for 45 min in 5% CO₂. At the end of the incubation period, unattached cells were removed by washing 3× with serum-free medium, and attached cells were counted using a Cytofluor 4000 flurometer (PE Applied Biosystems, Foster City, Calif.). The number of attached cells was represented as percentage of endothelial cell adhesion, as determined by the ratio of attached cells in the presence or absence of factor.

Results:

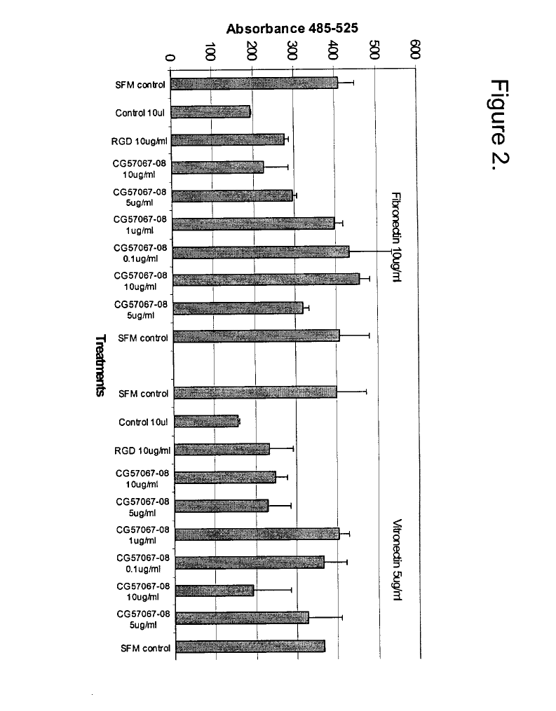

HUVEC cells adhered to all the ECM proteins, with maximum binding seen to fibronectin-coated plates. Addition of Angioarrestin, at different concentrations resulted in a dose-dependent inhibition of cell adhesion to ECM-coated plates. At 10 ug/ml there was a 80% reduction of HWEC cell binding to fibronectin-treated plates. (FIG. 2). Results showed that AngioarrestinFBD/Fc (CG57067-08) inhibits enldothelial cell adhesion to different ECM proteins.

### Example 6

Angioarrestin Inhibits MIGRATION of Endothelial and Carcinoma Cells

Migration Assay

To determine if Angioarrestin proteins CG57067 influence cell migration, cell lines were screened for cell motility in response to various treatments. Cell lines tested include: HUVEC (human umbilical vein endothelial cells), Panc-1 (pancreatic carcinoma), U87MG (glioblastoma), 786-0 (renal carcinoma, epithelial), HT1080 (fibrosarcoma), A549 and NCI-H1299 (lung carcinoma), OVCA5 (ovarian carcinoma), MDA-MB-468 (breast carcinoma), and CCD1070 (foreskin fibroblast). The bottom of the wells of the culture plate, or lower chamber contained purified CG57067 in the presence or absence growth factors such as VEGF or PDGF Collagen I coated insert membranes (Becton Dickinson) were prepared by rehydration for at least 30 minutes at room temperature with 300 μL of basal media and placed in the culture wells over the lower chamber. Approximately 90% confluent T175 flask of cells was treated with 2 mL trypsin, neutralized with 10 mL neutralization solution and centrifuged for 7 minutes at 800 RPM. Cells were resuspended in 10 mL basal media containing 0.1%BSA (diluent) and spun again. Cells were resuspended in diluent, counted, and adjusted to 6×10⁴ cells/ml with diluent.and 3×10⁴ cells/well) were placed in culture wells suspended above a membrane and a lower chamber.. The cells were incubated for 6 to 24 h and then assayed quantitatively for migratory activity (Cascade Biologics), VEGF (10 ng/mL) was a positive control for motility factor for endothelial cells. After the indicated incubation period, the cells were removed from the upper side of the insert using a cotton swab. The cell adhering to the underside of the filter were stained with 0.2% crystal violet in 70% ethanol, washed with distilled water, and counted under the microscope.

Results:

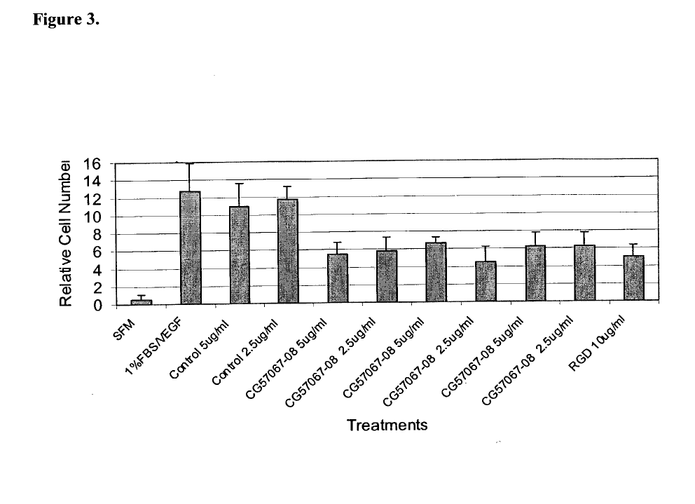

CG57067-08 inhibits endothelial cell migration. AngioarrestinFBD/Fc significantly inhibited the VEGF-induced migration of endothelial cells in a dose-dependent manner. A concentration of 2.5 ug/ml of Angioarrestin FBC/Fc resulted in approximately 40% inhibition of migration of HUVEC cells. (FIG. 3). These results demonstrate that Angioarrestin FBD/Fc specifically inhibits the VEGF-mediated migration of endothelial cells.

Results:

CG57067-19 Inhibits Cell Migration:

TABLE 3
HUVEC Cell Migration

| Treatment | Average Number of Cells | SD |
|---|---|---|
| Serum Free Media (SFM) | 1.33 | 1.54 |
| 1% FBS/VEGF | 28.00 | 2.00 |
| CG57067-19 0.5 uM | 2.00 | 2.00 |
| CG57067-19 0.25 uM | 3.33 | 0.58 |
| CG57067-19 0.125 uM | 6.66 | 1.53 |
| CG57067-19 0.0625 uM | 17.33 | 1.15 |
| RGD 10 ug/ml | 4.00 | 0.00 |

TABLE 4
Pancreatic carcinoma Panc-1 Cell Migration

| Treatment | Average Number of Cells | SD |
|---|---|---|
| Serum Free Media (SFM) | 0.33 | 0.58 |
| 2.5% FBS | 46.00 | 5.29 |
| CG57067-19 0.5 uM | 1.33 | o.58 |
| CG57067-19 0.25 uM | 6.67 | 2.31 |
| CG57067-19 0.125 uM | 12.67 | 4.16 |
| CG57067-19 0.0625 uM | 30.67 | 4.04 |
| RGD 10 ug/ml | 11.00 | 2.65 |
| Kinin dc 10 ug/ml | 8.33 | 2.52 |

TABLE 5
U87MG Glioblastoma cell migration

| Treatment | Average Number of Cells | SD |
|---|---|---|
| Serum Free Media (SFM) | 0.33 | 0.58 |
| 1% FBS | 32.34 | 4.04 |
| CG57067-19 0.5 uM | 1.33 | 0.58 |
| CG57067-19 0.25 uM | 5.67 | 1.15 |
| CG57067-19 0.125 uM | 12.33 | 3.21 |
| CG57067-19 0.0625 uM | 19 | 2.65 |
| RGD 10 ug/ml | 10.67 | 1.53 |

TABLE 6
HT1080 Fibrosarcoma cell migration

| Treatment | Average Number of Cells | SD |
|---|---|---|
| Serum Free Media (SFM) | 0.33 | 0.58 |
| 1% FBS | 37.67 | 2.52 |
| CG57067-19 1.0 uM | 0.33 | 0.57 |
| CG57067-19 0.5 uM | 0.33 | 0.57 |
| RGD 10 ug/ml | 11.67 | 2.08 |

TABLE 7
786-0 Renal Cell Carcinoma Cell Migration

| Treatment | Average Number of Cells | SD |
|---|---|---|
| Serum Free Media (SFM) | 0.33 | 0.58 |
| 1% FBS | 26.33 | 2.52 |
| CG57067-19 0.5 uM | 0.33 | 0.58 |
| CG57067-19 0.25 uM | 3.00 | 1.73 |
| CG57067-19 0.125 uM | 11.67 | 4.93 |
| CG57067-19 0.0625 uM | 19.00 | 1.00 |
| RGD 10 ug/ml | 2.00 | 1.00 |

TABLE 8
A549 Lung carcinoma cell migration

| Treatment | Average Number of Cells | SD |
|---|---|---|
| Serum Free Media (SFM) | 0.33 | 0.58 |
| 1% FBS | 15.00 | 4.36 |
| CG57067-19 0.5 uM | 1.00 | 0.00 |
| CG57067-19 0.25 uM | 7.67 | 3.06 |
| CG57067-19 0.1 uM | 14.00 | 2.65 |
| CG57067-19 0.01 uM | 16.00 | 3.00 |
| RGD 10 ug/ml | 10.33 | 4.16 |

TABLE 8A
Compared to CG57067-08:

| Treatment | Average Number of Cells | SD |
|---|---|---|
| % FBS | 14.00 | 6.24 |
| CG57067-08 1.0 uM | 16.00 | 4.58 |
| CG57067-08 0.5 uM | 16.67 | 5.03 |
| CG57067-08 0.25 uM | 15.67 | 5.13 |
| Kinin dc 10 ug/ml | 4.00 | 2.65 |

TABLE 9
NCI-H1299 Lung carcinoma cell migration

| Treatment | Average Number of Cells | SD |
|---|---|---|
| Serum Free Media (SFM) | 0.33 | 0.58 |
| 1% FBS/VEGF | 18.67 | 2.08 |
| CG57067-19 1.0 uM | 0.33 | 0.58 |
| CG57067-19 0.5 uM | 0.33 | 0.58 |
| RGD 10 ug/ml | 3.67 | 0.58 |

TABLE 10
OVCAR5 Ovarian Cancer Cell Migration

| Treatment | Average Number of Cells | SD |
|---|---|---|
| Serum Free Media (SFM) | 0.33 | 0.58 |
| 1% FBS | 49.00 | 2.65 |
| CG57067-19 0.5 uM | 28.67 | 9.07 |
| CG57067-19 0.25 uM | 48.67 | 5.29 |
| CG57067-19 0.1 uM | 48.00 | 5.29 |
| CG57067-19 0.01 uM | 45.00 | 5.00 |
| RGD 10 ug/ml | 12.33 | 4.04 |

TABLE 11
MDA-MB-468 Breast Carcinoma Cell Migration

| Treatment | Average Number of Cells | SD |
|---|---|---|
| Serum Free Media (SFM) | 106.5 | 0.71 |
| 3% FBS | 329.5 | 9.19 |
| CG57067-19 1 uM | 170.5 | 0.71 |
| CG57067-19 0.5 uM | 201.00 | 4.24 |
| CG57067-19 0.25 uM | 205.00 | 1.41 |
| CG57067-19 0.10 uM | 226.5 | 26.16 |
| CG57067-19 0.01 uM | 310.00 | 18.38 |
| RGD 30 ug/ml | 218.5 | 7.78 |

### Example 7

Angioarrestin CG57067-08 Inhibition of 786-0 Renal Cell Carcinoma Induced Angiogenesis in Vivo.

Single cell suspension of human renal clear cell asenocarcinoma cell line 786-0 cultures were prepared by trypsinization (5 min). Cells were mixed with Matrigel preparation to achieve a final cell density of 2×10⁶/ml. Matrigel containing 786-0 cell mixtures was distributed into five 50-ml, sterile culture tubes. A control matrigel solution without any cells was prepared separately (Group 1).

Out of the five tubes containing 786-0 cells, two tubes received vehicle alone. One of these two preparations was used as a positive control (Group 2). The other preparation was used for in vivo parenteral treatment with CG57067-08 (Group 6). Remaining three tubes received varying concentrations of CG57067-08 (1, 5 and 50 ug/ml final concentration in the gel mixture)—experimental groups 3,4 and 5 respectively.

Female, athymic nude mice (nu/nu) 8 weeks old, were injected sub-cutaneously on the right flank with 0.5 ml of a matrigel mixture. Six groups of mice were used. Each group had five mice.

Group 1: Matrigel alone

Group 2: Matrigel+786-0 cells (2×10⁶/ml)+vehicle

Group 3: Matrigel+786-0 cells (2×10⁶/ml)+CG57067-08, 1 ug/ml

Group 4: Matrigel+786-0 cells (2×10⁶/ml)+CG57067-08, 5 ug/ml

Group 5: Matrigel+786-0 cells (2×10⁶/ml)+CG57067-08, 50 ug/ml

Group 6: Matrigel+786-0 cells (2×10⁶/ml)+vehicle+intraperitoneal treatment with CG57067-08, −5.0 mg/kg, twice daily for 7 days.

Stock matrigel preparations were made for each group and 0.5 ml of the suspension was injected per mouse, subcutaneously, under aseptic conditions. Body weight measurements were made using an electronic balance.

The matrigel implants solidified in situ and were left undisturbed for 7 days. At the end of 7 days, mice were anesthetized by Ketamine and Xylazine mixture, and the matrigel plugs were removed carefully using microsurgical instruments. Gels were photographed under transillumination. One part of the gel was then fixed in buffered 10% formaldehyde (Sigma Chemicals) overnight and processed for paraffin embedded sectioning. Sections were cut at three different levels and stained with H/E. Another part of the gel was snap frozen in liquid nitrogen and then 10 □m sections of were prepared. Frozen sections were used for immunocytochemical staining with rat monoclonal antibody directed against mouse CD31 antigen conjugated with phycoerythrin (Pharmingen). DAPI staining was used to identify nucleated cells infiltrating the Matrigel plugs. H+E stained slides were evaluated for the formation of distinct, endothelial lined capillaries. Anti-CD31-PE stained slides were observed under Fluorescence microscope using appropriate filters. Images were captured digitally using Metamorph software program. Same areas were photographed under red and UV filters to acquire images from CD-31 PE and DAPI staining. Microvessel density was determined by the method published by Wild et al. (2000). DAPI images were superimposed with respective CD31-PE images to localize blood vessels.

Results:

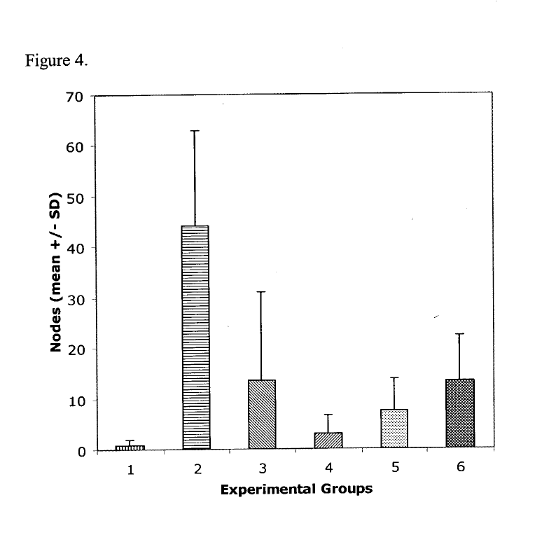

Results show the gross morphology of matrigels resected from mice. (FIG. 4). Group 1, control, showed no visible angiogenesis. Gels were essentially transparent and soft.

Matrigels from Group 2 showed evidence of angiogenesis. All the five gels showed hemorrhagic spots. Matrigels from Groups 2 to 6 were solid in contrast to matrigel plugs from the negative control group, 1. Groups 3-6 showed decreased vascularity. Some of the matrigel plugs from groups 3,4 and 5 showed evidence of hemorrhage.

Histology

Histology of control matrigels showed no major vessels. Most of the areas remained clear with a few layers of infiltrating nucleated cells. Histology of 786-0 matrigels showed cell-induced changes in the matrigels. Group 2 gels showed clear evidence of vascularization. Mature blood vessels are frequently seen. Most of the areas showed well-organized viable tumor cells. Histology of matrigels from Group 3 where GC57067-08 (1.0 ug/ml) was included in these gels along with 786-0 cells. Sections showed reduced vascularity. Matrigels showed viable tumor cells well organized. Histology of matrigels from Group 4 in which CG57067-08 was added at 5.0 ug/ml showed two matrigels had no blood vessels. Other images showed several small blood vessels filled with RBC. Except for one section, all the other sections contained well-organized viable tumor cells. Histology of matrigels from Group 5 where the highest concentration of CG57067-08 (50 ug/ml) was used with 786-0 cells showed major blood vessels in one gel. Other gels contained no major blood vessels. Tumor cell density was also reduced in these sections. Histology of matrigels from Group 6, which was parenterally treated with 5.0 mg/kg of CG57067-08 showed no major areas of vascularity. Tumor cell density was less when compared to the positive control group. Areas of viable tumor cells were dispersed among necrotic tumor cells.

CD31 staining of Group 1 matrigels showed no major vascularized areas. Nucleated cells are seen primarily at the periphery of matrigel plugs. In comparison, matrigel sections from Group 2 showed distinct blood vessel staining dispersed among DAPI stained nucleated cells. In matrigels treated with CG57067-08 (Groups 3, 4 and 5), some of the sections from the Group 3 showed highly vascularized areas. Staining from Group 4 (in situ treatment with 5.0 ug/ml of CG57067-08) showed no major areas of staining. However one section did show a well vascularized area dispersed evenly among the nucleated cells (blue). Group 5. matrigel plugs contained 50 ug/ml of CG57067-08. Except for one, all sections contained only isolated areas of CD-31 positive staining. Most of the areas remained free of blood vessels. Group 6 was treated parenterally with 5.0 mg/kg of CG57067-08. Density of nucleated cells is reduced in most sections, showing markedly reduced number of CD31-positive blood vessels.

Morphometric Analysis

Data from Morphometric analyses is shown in Table 12. Vessel nodes, ends and length from individual gels are tabulated. Mean number of vessel ends, nodes and length are shown. P value was calculated between group 1/2; Group 2/3; Group 2/4; Group 2/5 and Group 2/6. Data in FIG. 4 show comparative angiogenic response (number of nodes) in individual groups. Control group showed a mean of 0.92 node per unit area. Presence of 786-0 cancer cells in the gels stimulated neovascularization. Mean number of nodes increased to 44.18 with a range between 19.5 and 61. When CG57067-08 was added to the gels at a concentration of 1.0 ug/ml along with 786-0 cells, there was a reduction in angiogenic response. About 70.9% decrease in the number of nodes was observed (range 1-47). Increasing the concentration to 5.0 ug/ml reduced the number of nodes further. Highest inhibition (95%) was seen in this group (range 0.16-9.6 nodes). At the highest concentration tested (50 ug/ml of CG57067-08), there was 84.56% inhibition of angiogenic response (number of nodes 7.5+/−6.28). P values showed statistically significant inhibition (<0.05) of angiogenesis by CG57067-08.

TABLE 12

| | A | B | C | D | E | Mean | SD | p | P2 |
|---|---|---|---|---|---|---|---|---|---|
| Nodes | | | | | | | | | |
| 1 | 1 | 0.2 | 2.8 | 0.6 | 0 | 0.92 | 1.00 | 0.004 | |
| 2 | 61 | 19.5 | 49.4 | 65.6 | 25.4 | 44.18 | 18.61 | | |
| 3 | 4.5 | 1.2 | 47 | 14 | 1 | 13.54 | 17.38 | | 0.020 |
| 4 | 0.167 | 9.6 | 0.33 | 4 | 1.4 | 3.0994 | 3.53 | | 0.008 |
| 5 | 7 | 8 | 19 | 0.6 | 3.4 | 7.6 | 6.28 | | 0.011 |
| 6 | 14.6 | 16 | 28 | 2 | 6 | 13.32 | 9.01 | | 0.022 |
| Ends | | | | | | | | | |
| 1 | 14 | 4.2 | 10 | 12.2 | 6.8 | 9.44 | 3.55 | 0.004 | |
| 2 | 279 | 139 | 120 | 184 | 98 | 164 | 64.10 | | |
| 3 | 22 | 6.4 | 230 | 34 | 12 | 60.88 | 85.08 | | 0.081 |
| 4 | 15 | 113 | 10.3 | 170 | 33 | 68.26 | 62.92 | | 0.051 |
| 5 | 42 | 39 | 112 | 14 | 25 | 46.4 | 34.31 | | 0.021 |
| 6 | 51 | 70 | 97 | 28 | 31 | 55.4 | 25.73 | | 0.021 |
| Length | | | | | | | | | |
| 1 | 1.3 | 10.2 | 1.17 | 0.34 | 0.19 | 2.64 | 3.81 | 0.011 | |
| 2 | 31 | 11 | 23 | 25 | 15 | 21 | 7.16 | | |
| 3 | 1.32 | 0.26 | 27.7 | 5.4 | 0.56 | 7.048 | 10.49 | | 0.034 |
| 4 | 0.36 | 7.5 | 0.43 | 9.16 | 1.11 | 3.712 | 3.82 | | 0.009 |
| 5 | 3 | 6 | 13 | 1.6 | 1 | 4.92 | 4.39 | | 0.010 |
| 6 | 4 | 8 | 12 | 3 | 3.2 | 6.04 | 3.49 | | 0.012 |

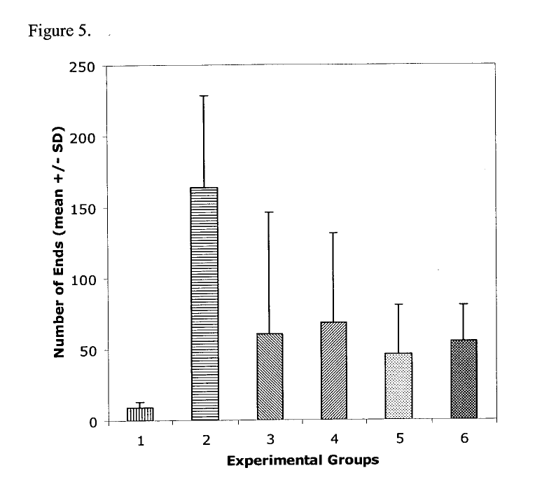

Data in FIG. 5 show the relative number of vessel ends. Control gels (Group 1) had a mean number of 9.44 vessel ends. 786-0 cells increased the number of vessels by 17.37-fold (mean number of ends 164.0). Number of vessel ends reduced in the presence of CG57067-08. At 1.0 ug/ml vessel end reduced to 66.7% when compared to the positive control group (2). Huge variations were reflected in the SD. Mean number of ends ranged between 6.4 and 230. P value did not show any statistical significance in the reduction of vessel ends at 1.0 ug/ml concentration. At 5.0 ug/ml concentration vessel ends were reduced to 62.0%. This group again showed large variations and the p value was 0.051. At the highest concentration tested (50 ug/ml) however showed significant decrease in the number of blood vessels (76% inhibition, P value=0.021). Parenteral administration of CG57067-08 resulted in statistically significant inhibition in vessel ends (70.27% inhibition when compared to group 2).

To calculate % inhibition of angiogenesis by CG57067-08, following method was used. Values from Group 2-Values from Group 1×100 is considered as 100% angiogenic response. Values from other experimental Groups i.e., 3, 4, 5 and 6 were subtracted from negative control values (Group 1) before calculating the level of inhibition.

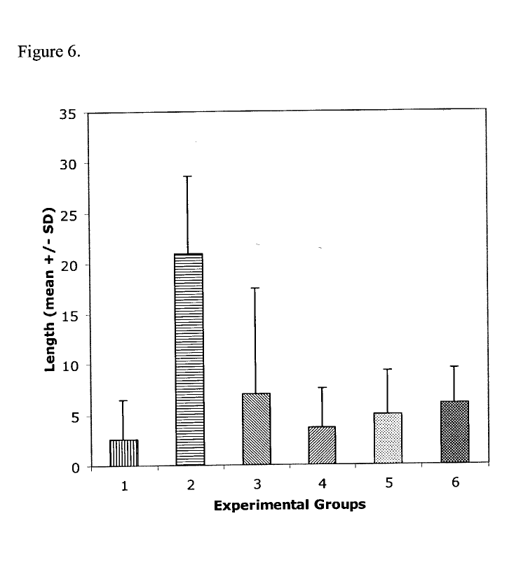

FIG. 6 shows the relative length of blood vessels from each group. Compared to control group, 1, 786-0 cells containing gels (Group 2) showed a 8-fold increase in total vessel length (2.64 Vs 21). This batch of negative control had some major blood vessels. Normally, the negative control groups show values <1. 786-0 induced changes in the vessel length were comparable to previous experiments. Inclusion of CG57067-08 at all the three concentrations tested (1, 5, 50 ug/ml) inhibited total vessel length significantly. For example at a concentration of 1.0 ug/ml there was 76% inhibition in total vessel length. At 5 ug/ml there was maximum inhibition (94.23%). Between treatment groups there was no statistical difference. Matrigels from parenterally treated group (6) showed significant reduction in vessel length (81.49% inhibition).

786-0 cancer cells induced significant angiogenic response. There was statistically significant increase in number of vessel ends, nodes and total length in 786-0 containing gels when compared to negative controls (matrigel alone). CD31 staining and histology showed reduced vascularity in CG57067 containing matrigel plugs. Morphometric analyses show CG57067-08 inhibited 786-0 cancer cell-induced angiogenesis significantly at the highest dose. Number of vessel ends did not show significant inhibition at 1.0, 5.0 ug/ml concentrations. Vessel nodes and length however had significant inhibition at lower concentrations as well. Parenteral administration of CG57067-08 inhibited 786-0 renal carcinoma cell-induced angiogenesis significantly.

### Example 8

Angioarrestin CG57067-19 Inhibition of 786-0 Renal Cell Carcinoma Induced Angiogenesis in Vivo.

Renal carcinoma cell line, 786-0 was used as angiogenic signal to induce neovascularization in matrigel plug assay. Single cell suspension was prepared on the day of experiment by trypsinization. Viability was routinely determined by trypan blue dye exclusion. Viability of the cells were >99% of the total cell suspension.

Stock matrigel preparation (total volume, 15.0 ml) containing 786-0 (2×10⁶/ml) was made in a 50-ml, sterile culture tube. As a negative control, 4.62 ml of matrigel solution from the same batch was prepared without any cells. From the stock solution, 0.5 ml of the suspension was injected per mouse, subcutaneously, under aseptic conditions. Negative control group received equal volume of Matrigel plus vehicle alone. Female, athymic nude mice (nu/nu) 8 weeks old were used in this study. Each group had five mice.

Group 1: Matrigel alone

Group 2: Matrigel+786-0 cells

Group 3: Matrigel+786-0 cells (mice were treated i.p with CG57067-19, 1.0 mg/kg)

Group 4: Matrigel+786-0 cells (mice were treated i.p with CG57067-19, 5.0 mg/kg)

Group 5: Matrigel+786-0 cells (mice were treated i.p with CG57067-19, 10.0 mg/kg)

The matrigel implants solidified in situ and were left undisturbed for 7 days. Mice were weighed using an electronic balance. Group 2 mice were treated with 0.2 ml of vehicle, i.p., twice daily (8.0-9.0 AM and 6-7.0 PM). Treatment groups, 3, 4 and 5 received 1.0, 5.0 or 10.0 mg/kg of CG57067-19 twice daily, i.p. CG57067-19 was provided by CuraGen at a concentration of 1.8 mg/ml. Stock solution was diluted aseptically using sterile, HBSS. According to body weight the volume of injection was adjusted to achieve indicated dosage. At the end of 7 days, mice were anesthetized by Ketamine and Xylazine mixture, and the matrigel plugs were removed carefully using microsurgical instruments. Gels were photographed under transillumination. One part of the gel was then fixed in buffered 10% formaldehyde (Sigma Chemicals) overnight and processed for paraffin embedded sectioning. Sections were cut at three different levels and stained with H/E. Another part of the gel was snap frozen in liquid nitrogen and then 10 □m sections of were prepared. Frozen sections were used for immunocytochemical staining with rat monoclonal antibody directed against mouse CD31 antigen conjugated with phycoerythrin. DAPI staining was used to identify nucleated cells infiltrating the Matrigel plugs. H+E stained slides were evaluated for the formation of distinct, endothelial lined capillaries. Anti-CD31-PE stained slides were observed under Fluorescence microscope using appropriate filters. Images were captured digitally using Metamorph software program. Same areas were photographed under red and UV filters to acquire images from CD-31 PE and DAPI staining. Microvessel density was determined by the method published by Wild et al. (2000). DAPI images were superimposed with respective CD31-PE images to localize blood vessels.

Results

Gross morphology of matrigels resected from mice showed control gels (Group 1) were thin and soft. Gels were transparent and occasional surface vessels are observed. Group 2 matrigels showed vascularized regions. Gels were thick and hard perhaps due to the presence of tumor cells. Treatment groups, 3-5 showed varying degree of hemorrhage and vascularization. Matrigel plugs from these groups were again thicker and harder resembling positive control group. Two the gels in group 3 (3A and 3E), one from Group 4 (4A) and two from group 5 (5A and 5E) had localized hemorrhage. Two of the gels in group 3, four in group 4 and three in group 5 had lesser density of blood vessels based on their appearance.

Histology:

Histology of control matrigels showed no major blood vessels. Most of the areas remained clear. Infiltration of nucleated cells was minimum and restricted to a few layers at the periphery. Histology of 786-0-induced changes in the matrigels (Group 2). showed many large and small blood vessels. Healthy tumor cells are widely distributed as well organized clusters. Group 3, treated, with CG57067-19 at 1.0 mg/kg dose, given i.p. twice daily showed viable tumor cells. Localized regions of high vessel density are noticed. For example, 3D shows an area of high vessel density, which coincided with higher number of tumor cells. Other sections showed a few vessels per field (3A and 3E).

Group 4. animals were treated with CG57067-19 at a dose of 5.0 mg/kg, twice daily. 4D shows an area of high vessel density. Vessels were smaller in size when compared to positive control group. Group 5. animals were treated with the highest dose of CG57067-19, 10.0 mg/kg. Some histological changes in the 786-0 cells are seen. 5B and 5D for example had large areas of apoptotic cells. Cell density was lower than other groups. Some images showed hemorrhagic spots.

No CD31 positive vessels can be seen in sections from Group 1. Nucleated cells (DAPI staining) were minimum. In comparison, matrigel sections from Group 2 showed dramatic increase in vessel density indicating angiogenic response. Sections from Groups 3 showed significant amounts of vessel staining. Images from Group 4 show reduced vessel density. Group 5 had markedly reduced vessel staining when compared to other groups (Groups 2, 3, 4). Two of the images 5B and 5E show no CD31-positive cells.

Morphometric Analysis

Data from Morphometric analyses is shown in Table 13. Vessel nodes, ends and length from individual gels are tabulated. Mean number of vessel ends, nodes and length are shown. P values were calculated between group 1 Vs 2, 2 Vs 3, 2 Vs 4 and 2 Vs 5.

TABLE 13

| | Ends | Nodes | Length |
|---|---|---|---|
| 1A | 10.5 | 0.17 | 0.32 |
| 1B | 31.43 | 7.14 | 2.32 |
| 1C | 18.33 | 4.5 | 1.55 |
| 1D | 14 | 0.33 | 0.15 |
| 1E | 9.57 | 2.14 | 0.85 |
| Mean | 16.77 | 2.86 | 1.04 |
| STD | 8.9 | 3.0 | 0.9 |
| 2A | 1067 | 456 | 97.7 |
| 2B | 653 | 218 | 52.7 |
| 2C | 912 | 226 | 66.27 |
| 2D | 494 | 74 | 32.6 |
| 2E | 361 | 132 | 40.23 |
| MEAN | 697.4 | 221.2 | 57.9 |
| STD | 291.19 | 145.59 | 25.66 |
| P 1 vs 2 | 0.003 | 0.014 | 0.004 |
| 3A | 297.7 | 127.1 | 27.56 |
| 3B | 378.4 | 126 | 32.9 |
| 3C | 203.8 | 90 | 19.36 |
| 3D | 157.7 | 36.75 | 14.69 |
| 3E | 448.7 | 112.1 | 31.21 |
| MEAN | 297.26 | 98.39 | 25.144 |
| STD | 120.14 | 37.56 | 7.83 |
| P 2 vs 3 | 0.03 | 0.046 | 0.02 |
| 4A | 376 | 102.5 | 30.56 |
| 4B | 455 | 110.5 | 31.87 |
| 4C | 338 | 46.2 | 16.96 |
| 4D | 273 | 87.5 | 19.67 |
| 4E | 76.8 | 5 | 1.47 |
| MEAN | 303.76 | 70.34 | 20.106 |
| STD | 142.93 | 44.14 | 12.30 |
| P 2 vs 4 | 0.009 | 0.032 | 0.009 |
| 5A | 456 | 54.9 | 27.7 |
| 5B | 120.6 | 39.7 | 10.72 |
| 5C | 278.4 | 92.3 | 22.1 |
| 5D | 157.4 | 55.5 | 13.8 |
| 5E | 157.3 | 33.3 | 8.4 |
| MEAN | 233.94 | 55.14 | 16.54 |
| STD | 137.72 | 22.89 | 8.11 |
| P 2 vs 5 | 0.003 | 0.031 | 0.004 |

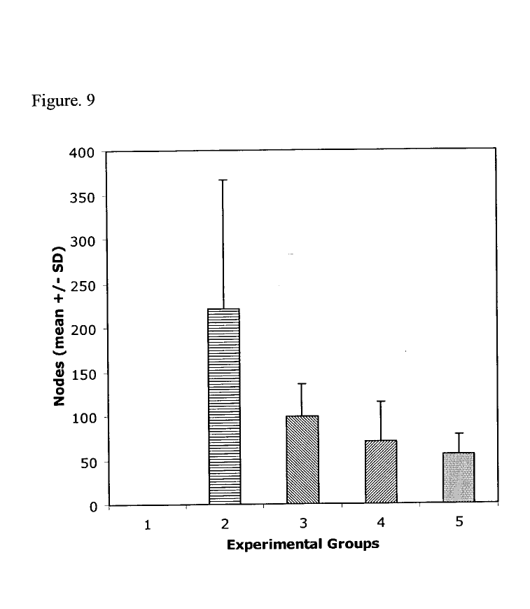

Data in FIG. 9 show comparative angiogenic response (number of nodes) in individual groups. Control group showed a mean number of 2.86 nodes per unit area. Inclusion of 786-0 cells in the gels stimulated neovascularization. Number of nodes increased to 221.2 (a 77.2-fold increase). When CG57067-19 was administered to mice, cancer cell-induced vascularization was inhibited significantly. At 1.0 mg/kg dose, there was a 56.25% reduction (P=0.046) in the number of nodes. Increasing the dose to 5.0 mg/kg resulted in 68.77% inhibition (P=0.032). At 10.0 mg/kg dose maximum inhibition in number of vessel nodes was seen (76.06%). P values between group 2 Vs group 5 was significant (0.031). These data indicate that CG57067-19 treatment inhibited 786-0-induced angiogenesis (nodes) in a dose dependent manner.

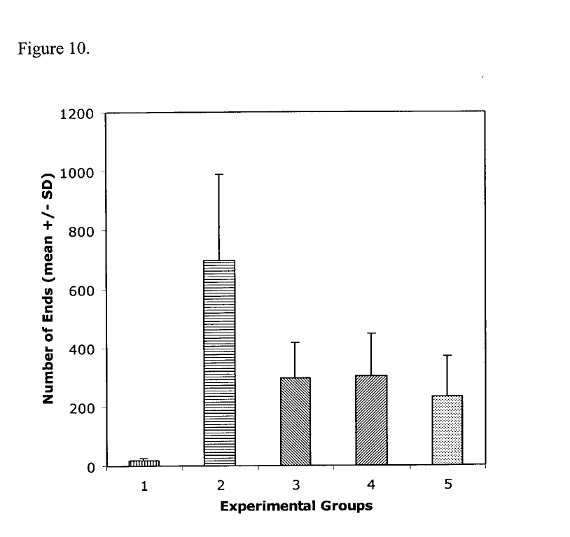

Data in FIG. 10 show the relative number of vessel ends. Control gels (Group 1) had a mean number of 16.77 vessel ends. 786-0 cells increased the number of vessel ends by 41.58-fold (697.4). Treatment with CG57067-19 reduced the number of vessel ends significantly in all the three doses tested. At 1.0 mg/kg dose, vessel ends were reduced by 58.79% (P=0.03) and at 5.0 mg/kg dose, 57.84% inhibition was seen (P=0.009). At the highest dose there was a further reduction in vessel ends. At 10.0 mg/kg dose level, vessel ends were reduced by 68.1% when compared to the positive control Group 2 (P=0.003).

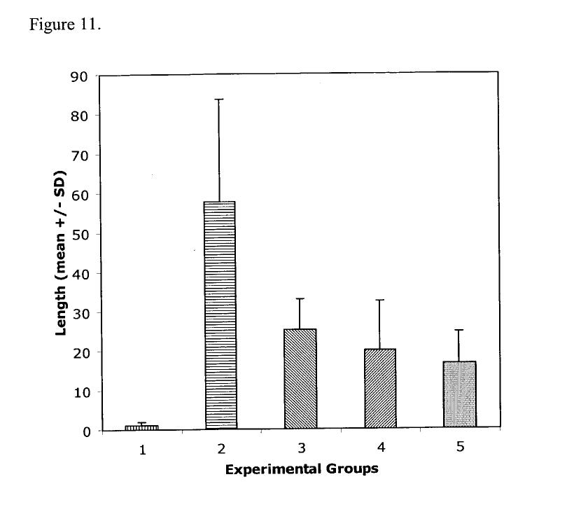

FIG. 11 shows the relative length of blood vessels from each group. Compared to control group, 786-0 cancer cell-containing gels showed a 55.67-fold increase in total vessel length (1.04 Vs 57.9). Mice treated with CG57067-19 showed inhibition in total vessel length in all the three doses tested. For example, injection of CG57067-19 at 1.0 mg/kg reduced the vessel length by 57.62% (P=0.02) when compared to the positive control (Group 2). At 5.0 mg/kg dose CG57067-19 treatment inhibited vessel length by 66.48% (P=0.009). CG57067-19 at 10 mg/kg showed further decrease in vessel length. At this dose level there was 72.75% inhibition when compared to positive control group, 2 (P=0.004).

Negative control matrigel plugs were clear and not vascularized. Inclusion of 786-0 renal carcinoma cells in matrigels elicited a good angiogenic response when compared to the negative control group. Vessel nodes, ends and length showed statistically significant increase. Histological analysis indicated large, mature blood vessels in 786-0 carcinoma cell-containing matrigel plugs. Matrigel plugs from mice treated with CG57067-19 showed areas of tumor cell necrosis/apoptosis at 10.0 mg/kg dose. Morphometric analyses show CG57067-19 treatment of mice significantly inhibited angiogenesis at all the three dose levels tested. Nodes, vessel ends and vessel length are all significantly inhibited by CG57067-19 treatment. There was a dose-dependent inhibition of angiogenic response.

### Other Embodiments

Although particular embodiments are disclosed herein in detail, this is done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims. The claims presented are representative of the inventions disclosed herein. Other, unclaimed inventions are also contemplated. Applicants reserve the right to pursue such inventions in later claims.

We claim:

1. An isolated polynucleotide selected from the group consisting of:
(a) a polynucleotide comprising the nucleotide sequence SEQ ID NO:73 encoding a Fibrinogen Binding Domain (FBD) and a polynucleotide comprising a fragment of the nucleotide sequence, SEQ ID NO:67 encoding a coiled coil (CC) domain;
(b) a polynucleotide comprising the nucleotide sequence SEQ ID NO:73 encoding a Fibrinogen Binding Domain (FBD) and a polynucleotide comprising a nucleotide sequence SEQ ID NO:69 encoding a coiled coil 1 (CC1) domain;
(c) a polynucleotide comprising the nucleotide sequence SEQ ID NO:73 encoding a Fibrinogen Binding Domain (FBD) and a polynucleotide comprising a nucleotide sequence SEQ ID NO:71, encoding a coiled coil 2 (CC2) domain;
(d) a polynucleotide encoding a protein comprising the amino acid sequence SEQ ID NOs: 2, 8, 10, 12, 18, 24, 26, 28, 30, 32, 34, 36, 38, 40, 42, 44, 46, 48, 50, 52, 54, 56, 58, 60, 62, 64, 70, 72;
(e) a polynucleotide encoding a protein comprising a fragment of the amino acid sequence SEQ ID NOs: 2, 8, 10, 12, 18, 24, 26, 28, 30, 32, 34, 36, 38, 40, 42, 44, 46, 48, 50, 52, 54, 56, 58, 60, 62, 64, having biological activity;
(f) a polynucleotide comprising the nucleic acid sequence of SEQ ID NOs: 1, 7, 9, 11, 17, 23, 25, 27, 29, 31, 33, 35, 37, 39, 41, 43, 45, 47, 49, 51, 53, 55, 57, 59, 61, 69, 71;
(g) a polynucleotide capable of hybridizing under stringent conditions to any one of the polynucleotides specified in (a)-(f).

2. An isolated polynucleotide selected from the group consisting of:
(a) a polynucleotide encoding the amino acid sequence SEQ ID NO:74 Fibrinogen Binding Domain (FBD) and a fragment of an amino acid sequence SEQ ID NO:68 coiled coil (CC) domain;
(b) a polynucleotide encoding the amino acid sequence SEQ ID NO:74 Fibrinogen Binding Domain (FBD) and an amino acid sequence SEQ ID NO: 70, coiled coil 1 (CC1) domain:
(c) a polynucleotide encoding the amino acid sequence SEQ ID NO:74 Fibrinogen Binding Domain (FBD) and an amino acid sequence SEQ ID NO:72 coiled coil 2 (CC2) domain;
(d) a polynucleotide encoding an amino acid sequence SEQ ID NOs: 2, 8, 10, 12, 18, 24, 26, 28, 30, 32, 34, 36, 38, 40, 42, 44, 46, 48, 50, 52, 54, 56, 58, 60, 62, 64, 70, 72;
(e) a polynucleotide encoding a protein comprising a fragment of the amino acid sequence SEQ ID NOs: 2, 8, 10, 12, 18, 24, 26, 28, 30, 32, 34, 36, 38, 40, 42, 44, 46, 48, 50, 52, 54, 56, 58, 60, 62, 64, having biological activity;
(f) a polynucleotide capable of hybridizing under stringent conditions to any one of the polynucleotides specified in (a)-(e).

3. An isolated polypeptide selected from the group consisting of:
(a) a polypeptide comprising the amino acid sequence SEQ ID NO:74 Fibrinogen Binding Domain (FBD) and an amino acid sequence comprising a fragment of the amino acid sequence SEQ ID NO:68 coiled coil (CC) domain;
(b) a polypeptide comprising the amino acid sequence SEQ ID NO:74 Fibrinogen Binding Domain (FBD) and an amino acid sequence comprising SEQ ID NO: 70, coiled coil 1 (CC1) domain;
(c) a polypeptide comprising the amino acid sequence SEQ ID NO:74 Fibrinogen Binding Domain (FBD) and an amino acid sequence comprising SEQ ID NO:72 coiled coil 2 (CC2) domain;
(d) a polypeptide comprising the amino acid sequence SEQ ID NOs: 2, 8, 10, 12, 18, 24, 26, 28, 30, 32, 34, 36, 38, 40, 42, 44, 46, 48, 50, 52, 54, 56, 58, 60, 62, 64, 70, 72;
(e) a polypeptide comprising a fragment of the amino acid sequence SEQ ID NOs: 2, 8, 10, 12, 18, 24, 26, 28, 30, 32, 34, 36, 38, 40, 42, 44, 46, 48, 50, 52, 54, 56, 58, 60, 62, 64, having biological activity;
(f) a polypeptide encoded by a polynucleotide capable of hybridizing under stringent conditions to any one of the polynucleotides encoding polypeptids specified in (a)-(e).

4. The polynucleotide of claim 1 wherein said polynucleotide is operably linked to an expression control sequence.

5. A cell comprising the polynucleotide of claim 1.

6. The cell of claim 5, wherein said cell is a mammalian cell.

7. A process for producing a protein, said process comprising providing the cell of claim 5 under conditions allowing for expression of a protein by said polynucleotide.

8. A protein comprising the amino acid sequence selected from the group consisting of SEQ ID NOs: 2, 8, 10, 12, 18, 24, 26, 28, 30, 32, 34, 36, 38, 40, 42, 44, 46, 48, 50, 52, 54, 56, 58, 60, 62, 64, 70, 72.

9. A process for producing a protein, which comprises (a) growing a culture of the host cell of claim 5 in a suitable culture medium; and (b) purifying the protein from the culture.

10. A method of inhibiting cell proliferation, comprising contacting said cell with the polypeptide of claim 3 or 8 in an amount sufficient to inhibit proliferation of said cell.

11. A method of inhibiting cell proliferation, comprising contacting said cell with the polynucleotide of claim 1 in an amount sufficient to inhibit proliferation of said cell.

12. The method of claim 10, wherein said cell is provided in vitro, ex vivo, or in vivo.

13. A method of inhibiting the growth of a tumor in a subject, the method comprising administering to said subject the polypeptide of claim 3 or 8 in an amount sufficient to inhibit the growth of said tumor.

14. A method of inhibiting the growth of a tumor in a subject, the method comprising administering to said subject an nucleic acid of claim 1 in an amount sufficient to inhibit the growth of said tumor.

15. The method of claim 13, wherein said nucleic acid is administered by a route selected from the group consisting of oral, intravenous, intramuscular, subcutaneous, or nasal delivery.

16. A method of inhibiting tumor metastasis in a subject, the method comprising administering to said subject an amount of the polypeptide of claim 3 or 8 in an amount sufficient to inhibit metastasis of said tumor.

17. The method of claim 14, wherein said polypeptide is administered by a route selected from the group consisting of oral, intravenous, intramuscular, subcutaneous, or nasal delivery.

18. A method of inhibiting tumor metastasis in a subject, the method comprising administering to said subject an amount of a nucleic acid encoding the polypeptide of claim 3 or 8 in an amount sufficient to inhibit metastasis of said tumor.

19. The method of claim 16, wherein said subject is a human.

20. A method of inhibiting angiogenesis in a subject, said method comprising administering to the mammal an amount of a polypeptide of claim 3 or 8 in an amount sufficient to inhibit angiogenesis.

21. A method of inhibiting angiogenesis in a subject, said method comprising administering to the mammal an amount of a nucleic acid encoding a polypeptide of claim 3 or 8 in an amount sufficient to inhibit angiogenesis.
